SEQUENCE LISTING (46 sequences)

SEQ ID NO: 1 - protein, 1463 aa, Homo sapiens
MLLSPSLLLLLLLGAPRGCAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHSIIPLAVVLTLIVIVAICTLSFCIYKHNGGFFRRLAGFRNPYYPATNFSTVYLEENILISDLEKSDQ

SEQ ID NO: 2 - protein, 124 aa, synthetic construct
KGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEY

SEQ ID NO: 3 - protein, 31 aa, synthetic construct
WQDKGIFVIQSESLKKCIQAGKSVLTLENCK

SEQ ID NO: 4 - protein, 118 aa, synthetic construct
NSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICK

SEQ ID NO: 5 - protein, 112 aa, synthetic construct
YGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAIC

SEQ ID NO: 6 - protein, 122 aa, synthetic construct
FKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICK

SEQ ID NO: 7-8 - skipped (2 entries)

SEQ ID NO: 9 - protein, 810 aa, Homo sapiens
MPMDLILVVWFCVCTARTVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 10 - protein, 1657 aa, Homo sapiens
MGLQARRWASGSRGAAGPRRGVLQLLPLPLPLPLLLLLLLRPGAGRAAAQGEAEAPTLYLWKTGPWGRCMGDECGPGGIQTRAVWCAHVEGWTTLHTNCKQAERPNNQQNCFKVCDWHKELYDWRLGPWNQCQPVISKSLEKPLECIKGEEGIQVREIACIQKDKDIPAEDIICEYFEPKPLLEQACLIPCQQDCIVSEFSAWSECSKTCGSGLQHRTRHVVAPPQFGGSGCPNLTEFQVCQSSPCEAEELRYSLHVGPWSTCSMPHSRQVRQARRRGKNKEREKDRSKGVKDPEARELIKKKRNRNRQNRQENKYWDIQIGYQTREVMCINKTGKAADLSFCQQEKLPMTFQSCVITKECQVSEWSEWSPCSKTCHDMVSPAGTRVRTRTIRQFPIGSEKECPEFEEKEPCLSQGDGVVPCATYGWRTTEWTECRVDPLLSQQDKRRGNQTALCGGGIQTREVYCVQANENLLSQLSTHKNKEASKPMDLKLCTGPIPNTTQLCHIPCPTECEVSPWSAWGPCTYENCNDQQGKKGFKLRKRRITNEPTGGSGVTGNCPHLLEAIPCEEPACYDWKAVRLGNCEPDNGKECGPGTQVQEVVCINSDGEEVDRQLCRDAIFPIPVACDAPCPKDCVLSTWSTWSSCSHTCSGKTTEGKQIRARSILAYAGEEGGIRCPNSSALQEVRSCNEHPCTVYHWQTGPWGQCIEDTSVSSFNTTTTWNGEASCSVGMQTRKVICVRVNVGQVGPKKCPESLRPETVRPCLLPCKKDCIVTPYSDWTSCPSSCKEGDSSIRKQSRHRVIIQLPANGGRDCTDPLYEEKACEAPQACQSYRWKTHKWRRCQLVPWSVQQDSPGAQEGCGPGRQARAITCRKQDGGQAGIHECLQYAGPVPALTQACQIPCQDDCQLTSWSKFSSCNGDCGAVRTRKRTLVGKSKKKEKCKNSHLYPLIETQYCPCDKYNAQPVGNWSDCILPEGKVEVLLGMKVQGDIKECGQGYRYQAMACYDQNGRLVETSRCNSHGYIEEACIIPCPSDCKLSEWSNWSRCSKSCGSGVKVRSKWLREKPYNGGRPCPKLDHVNQAQVYEVVPCHSDCNQYLWVTEPWSICKVTFVNMRENCGEGVQTRKVRCMQNTADGPSEHVEDYLCDPEEMPLGSRVCKLPCPEDCVISEWGPWTQCVLPCNQSSFRQRSADPIRQPADEGRSCPNAVEKEPCNLNKNCYHYDYNVTDWSTCQLSEKAVCGNGIKTRMLDCVRSDGKSVDLKYCEALGLEKNWQMNTSCMVECPVNCQLSDWSPWSECSQTCGLTGKMIRRRTVTQPFQGDGRPCPSLMDQSKPCPVKPCYRWQYGQWSPCQVQEAQCGEGTRTRNISCVVSDGSADDFSKVVDEEFCADIELIIDGNKNMVLEESCSQPCPGDCYLKDWSSWSLCQLTCVNGEDLGFGGIQVRSRPVIIQELENQHLCPEQMLETKSCYDGQCYEYKWMASAWKGSSRTVWCQRSDGINVTGGCLVMSQPDADRSCNPPCSQPHSYCSETKTCHCEEGYTEVMSSNSTLEQCTLIPVVVLPTMEDKRGDVKTSRAVHPTQPSSNPAGRGRTWFLQPFGPDGRLKTWVYGVAAGAFVLLIFIVSMIYLACKKPKKPQRRQNNRLKPLTLAYDGDADM

SEQ ID NO: 11 - protein, 60 aa, synthetic construct
TLYLWKTGPWGRCMGDECGPGGIQTRAVWCAHVEGWTTLHTNCKQAERPNNQQNCFKVCD

SEQ ID NO: 12 - protein, 28 aa, synthetic construct
GPGGIQTRAVWCAHVEGWTTLHTNCKQA

SEQ ID NO: 13 - DNA, 5633 bp, Homo sapiens
  source 1..5633: mol_type = other DNA
cccgagtgtcggttcactgtggagacagcggtggcggagtgggtctccagggctctgggctggcaaggcccccggaggggtggggcgcggaggaggctacagatccgcttccgcgcggcggggccgggtgcttgggacgcggctctgggctcccgggataaggggctcccgggacaaggggctcccggagagcccagtggttagcgatgctgctgtcgccgtcgctgctgctgctgctgctgctggggggcgccgcgggctgcgccgagggtgtggcggcggcgcttacccccgagcggctcctggagtggcaggataaaggaatatttgttatccaaagtgagagtctcaagaaatgcattcaagcaggtaaatcggttctgaccctcggtagaactggaagcaagcaagcaaacaagcacatgctgtggaaatgggtttcaaaccatggcctctttaacataggaggcagcggttgcctgggcctgaatttctccgccccagagcagccattaagcttatatgaatgtgactccaccctcgtttccttacggtggcgctgtaacaggaagatgatcacaggcccgctgcagtactctgtccaggtggcgcatgacaacacagtggtggcctcacggaagtatattcataagtggatttcttatgggtcaggtggtggagacatttgtgaatatctacacaaagatttgcatacaatcaaagggaacacccacgggatgccgtgtatgtttcccttccagtataaccatcagtggcatcatgaatgtacccgtgaaggtcgggaagatgacttactgtggtgtgccacgacaagccgttatgaaagagatgaaaagtggggattttgccctgatcccacctctgcagaagtaggttgtgatactatttgggagaaggacctcaattcacacatttgctaccagttcaacctgctttcatctctctcttggagtgaggcacattcttcatgccagatgcaaggaggtacgctgttaagtattacagatgaaactgaagaaaatttcataagggagcacatgagcagtaaaacagtggaggtgtgggttggcctcaatcagcttgatgaagacgctggctggcagtggtctgatggaacgccgctcaactatctgaattggagcccagaggtaaattttgagccatttgttgaagatcactgtggaacatttagttcatttatgccaagtgcctggaggagtcgggattgtgagtccaccttgccatatatatgtaaaaaatatctaaaccacattgatcatgaaatagttgaaaaagatgcgtggaaatattatgctacccactgtgagcctggctggaatccctacaatcgtaattgctacaaacttcagaaagaagaaaagacctggcatgaggctctgcgttcttgtcaggctgataacagtgcattaatagacataacctcattagcagaggtggagtttcttgtaaccctccttggagatgaaaatgcatcagaaacatggattggtttgagcagcaataaaattccagtttcctttgaatggtctaatgactcttcagtcatctttactaattggcacacacttgagccccacatttttccaaatagaagccagctgtgtgtctcagcagagcagtctgagggacactggaaagtcaaaaattgtgaagaaagacttttttacatttgtaaaaaagcaggccatgtcctctctgatgctgaatcaggatgtcaagagggatgggaggagacatgtggattctgttacaaaattgacacagtccttcgaagctttgaccaagcttccagcggttattactgtcctcctgcacttgtaaccattacaaacaggtttgaacaggcttttattaccagtttgatcagtagtgtggtaaaaatgaaggacagttatttttggatagctcttcaggaccaaaatgatacgggagaatacacttggaagccagtagggcagaaacccgagccggtgcagtacacacactggaacacacaccaaccgcgctacagtggtggctgtgttgccatgcgaggaaggcatccacttggtcgctgggaagtgaagcactgtcggcactttaaggcaatgtccttgtgcaagcagccagttgaaaatcaggaaaaagcagagtatgaagagagatggccctttcacccctgctatttggactgggagtcagagcctggtctggccagttgcttcaaggtatttcatagtgaaaaagttctgatgaaaagaacatggagagaagctgaagcattttgcgaagaatttggagctcatcttgcaagctttgcccatattgaggaagagaattttgtgaatgagctcttacacccaaaatttaattggacagaagaaaggcagttctggattggatttaataaaagaaacccactgaatgccggctcatgggagtggtctgatagaactcctgttgtctcttcgtttttagacaacacttattttggagaagatgcaagaaactgtgctgtttataagccaaacaaaacattgctgcccttacactgtggttccaaacgtgaatggatatgcaaaatcccaagagatgtgaaacccaagattccgttctggtaccagtacgatgtaccctggctcttttatcaggatgcagaatacctttttcatacctttgcctcagaatggttgaactttgagtttgtctgtagctggctgcacagtgatcttctcacaattcattctgcacatgagcaagaattcatccacagcaaaataaaagcgctatccaagtatggtgcaagttggtggattggacttcaagaagaaagagccaatgatgaatttcgctggagagatggaacaccagtgatataccagaactgggacacaggaagagaaagaactgtgaataatcagagccagagatgtggctttatttcttctataacaggactctggggtagtgaagagtgttcagtttctatgcctagtatctgtaagcgaaaaaaggtttggctcatagagaaaaagaaagatacaccaaaacaacatggaacgtgtcccaaaggatggctatattttaactataagtgccttctgctgaatatccccaaagacccaagcagttggaagaactggacgcatgcccaacatttctgtgctgaagaaggggggaccctggtcgccattgaaagtgaggtggagcaagctttcattactatgaatctttttggccagaccaccagtgtgtggataggtttacaaaatgatgattatgaaacatggctaaatggaaaacctgtggtatattctaactggtctccatttgatataataaatattccaagtcacaataccactgaagttcagaaacacattcctctctgtgccttactctcaagtaatcctaattttcatttcactggaaaatggtattttgaagactgtggaaaggaaggctatgggtttgtttgtgaaaaaatgcaagatacttctggacacggtgtaaatacatctgatatgtatccaatgcccaataccttagaatatggaaacagaacttacaaaataattaatgcaaatatgacttggtatgcagcaataaaaacctgcctgatgcacaaagcacaactggtcagcatcacagaccagtatcaccagtccttcctcactgttgtcctcaaccggctaggatatgcccactggattggactgttcaccacagataatggtcttaattttgactggtctgatggcaccaaatcttctttcactttttggaaagatgaggagtcctccctccttggtgactgcgtttttgccgacagcaacggacgctggcatagcacagcctgcgactcatttctgcaaggtgccatttgtcatgtaccacctgaaacaagacaatctgaacacccagagttgtgctcagaaacatctattccctggataaaatttaaaagtaattgctacaagttttctacagtcctagacagtatgagttttgaggctgctcatgaattttgcaaaaaggaaggttctaatcttttaacaatcaaggatgaggctgaaaatgcatttctcctagaagagctgtttgcttttggttcttctgtccagatggtttggttgaatgctcaatttgatggtaacaatgaaaccataaagtggtttgatggaactcccacagaccagtcaaactggggcattcggaagccagacacagactacttcaagccccatcattgtgttgccttgaggatccctgaaggattatggcagctatccccgtgtcaagaaaaaaaaggctttatatgtaaaatggaggcagatattcacactgcagaggcgctgccagaaaaaggaccaagtcacagcatcattcctcttgcggttgtactgacactgatagtcattgtggccatttgcacactttccttctgcatatacaagcataacggtggcttcttcaggagacttgcagggtttcggaatccttactatcctgcaaccaactttagtacagtatatttagaagaaaatattctcatttctgatcttgagaagagtgaccaataataatgaggtcagagaatgccacagacaccagggtaagtaaagaagactaaacaggagtctcatctgtctttccctttacagcacagatgccattagaatgtgaattgggtcactattttaattattcttgaagtgattactggttttgaatcttaaccaaatcagatgggttttgatttattcatttccctaaactgtgatccattcttaaaaggggtaaattatgcattggttatttttcagaaagacaagaactattaaaagaaactccctattgaaaactctgaaatcaatgcgaataatagtttttgcattaatgtatctctactaaaatttgggggaattttaaaactaatctggtatctattcagacatttacctgcactcgtaccattaagaaagacagaaagaagccaaaaaaaattaatcttgtatatgaggggaaaaggaaagggcttctgagaggattcttagttgtttcttttgaattccttttaatagcaggatttggaaaatactaatttctgtgcttaagggtcacaggttctgggctctcaactgatatttaaggtgacattcatttttattaggtctaacatctcaagctaaaggagaaagaaaaatacctccttttaaatggcaaagaccttcattagcagcacacttttataaacacccatatagttaaaatgtggccttaaactttcaattactaaatgatgattaagttggatattttaaaatgtcttatacataagcttaaagtaatatattgaaactttaacagttgtgctgtaaaaactcatggactttctgggattctaaatatattatataatatgttacactcttaataactggtagatataaaatgtaacttggatttaaaggagtagagctaaagatctgtattatagtctcattagtaccagacagatgttgttgagaagtactaaataaattagtaatctagatatccttattatgtaaatgagtttaggtgttctatttaataaactattttctgga

SEQ ID NO: 14 - protein, 20 aa, Homo sapiens
MLLSPSLLLLLLLGAPRGCA

SEQ ID NO: 15 - protein, 47 aa, Homo sapiens
MGLQARRWASGSRGAAGPRRGVLQLLPLPLPLPLLLLLLLRPGAGRA

SEQ ID NO: 16 - DNA, 10655 bp, synthetic construct
  source 1..10655: mol_type = other DNA
attctgcgaggaatggcagcggcagcagcatccccgccagaggcggcggcggcggcggcagcggccacggccaccaccgcggacgctattgttccctggtgttagacgcgctctccctccttctcatctgaagcgaacaatagcaggaaagagctttgctttctgtactctttgaaaagacgttccaagagcggagaaaaattccctgccgagcgtgctacggctctggaccctggagtggctgcaggcggcatggggctgcaagccaggcgctgggcgtccgggagccggggcgctgcggggccgcgccggggcgtcctgcagctgctgccgctgccgctgccgctgccgctgctcctgctgctgctgctacgcccgggcgccggcagggctgcggcgcagggcgaggcggaggcgcccaccctctatctgtggaagactggtccatggggccgatgtatgggagatgaatgtggtcccggaggcatccaaacgagggctgtgtggtgtgctcatgtggagggatggactacactgcatactaactgtaagcaggccgagagacccaataaccagcagaattgtttcaaagtttgcgattggcacaaagagttgtacgactggagactgggaccttggaatcagtgtcagcccgtgatttcaaaaagcctagagaaacctcttgagtgcattaagggggaagaaggtattcaggtgagggagatagcgtgcatccagaaagacaaagacattcctgcggaggatatcatctgtgagtactttgagcccaagcctctcctggagcaggcttgcctcattccttgccagcaagattgcatcgtgtctgaattttctgcctggtccgaatgctccaagacctgcggcagcgggctccagcaccggacgcgtcatgtggtggcgcccccgcagttcggaggctctggctgtccaaacctgacggagttccaggtgtgccaatccagtccatgcgaggccgaggagctcaggtacagcctgcatgtggggccctggagcacctgctcaatgccccactcccgacaagtaagacaagcaaggagacgcgggaagaataaagaacgggaaaaggaccgcagcaaaggagtaaaggatccagaagcccgcgagcttattaagaaaaagagaaacagaaacaggcagaacagacaagagaacaaatattgggacatccagattggatatcagaccagagaggttatgtgcattaacaagacggggaaagctgctgatttaagcttttgccagcaagagaagcttccaatgaccttccagtcctgtgtgatcaccaaagagtgccaggtttccgagtggtcagagtggagcccctgctcaaaaacatgccatgacatggtgtcccctgcaggcactcgtgtaaggacacgaaccatcaggcagtttcccattggcagtgaaaaggagtgtccagaatttgaagaaaaagaaccctgtttgtctcaaggagatggagttgtcccctgtgccacgtatggctggagaactacagagtggactgagtgccgtgtggaccctttgctcagtcagcaggacaagaggcgcggcaaccagacggccctctgtggagggggcatccagacccgagaggtgtactgcgtgcaggccaacgaaaacctcctctcacaattaagtacccacaagaacaaagaagcctcaaagccaatggacttaaaattatgcactggacctatccctaatactacacagctgtgccacattccttgtccaactgaatgtgaagtttcaccttggtcagcttggggaccttgtacttatgaaaactgtaatgatcagcaagggaaaaaaggcttcaaactgaggaagcggcgcattaccaatgagcccactggaggctctggggtaaccggaaactgccctcacttactggaagccattccctgtgaagagcctgcctgttatgactggaaagcagtgagactgggaaactgcgagccagataacggaaaggagtgtggtccaggcacgcaagttcaagaggttgtgtgcatcaacagtgatggagaagaagttgacagacagctgtgcagagatgccatcttccccatccctgtggcctgtgatgccccatgcccgaaagactgtgtgctcagcacatggtctacgtggtcctcctgctcacacacctgctcagggaaaacgacagaagggaaacagatacgagcacgatccattctggcctatgcgggtgaagaaggtggaattcgctgtccaaatagcagtgctttgcaagaagtacgaagctgtaatgagcatccttgcacagtgtaccactggcaaactggtccctggggccagtgcattgaggacacctcagtatcgtccttcaacacaactacgacttggaatggggaggcctcctgctctgtcggcatgcagacaagaaaagtcatctgtgtgcgagtcaatgtgggccaagtgggacccaaaaaatgtcctgaaagccttcgacctgaaactgtaaggccttgtctgcttccttgtaagaaggactgtattgtgaccccatatagtgactggacatcatgcccctcttcgtgtaaagaaggggactccagtatcaggaagcagtctaggcatcgggtcatcattcagctgccagccaacgggggccgagactgcacagatcccctctatgaagagaaggcctgtgaggcacctcaagcgtgccaaagctacaggtggaagactcacaaatggcgcagatgccaattagtcccttggagcgtgcaacaagacagccctggagcacaggaaggctgtgggcctgggcgacaggcaagagccattacttgtcgcaagcaagatggaggacaggctggaatccatgagtgcctacagtatgcaggccctgtgccagcccttacccaggcctgccagatcccctgccaggatgactgtcaattgaccagctggtccaagttttcttcatgcaatggagactgtggtgcagttaggaccagaaagcgcactcttgttggaaaaagtaaaaagaaggaaaaatgtaaaaattcccatttgtatcccctgattgagactcagtattgtccttgtgacaaatataatgcacaacctgtggggaactggtcagactgtattttaccagagggaaaagtggaagtgttgctgggaatgaaagtacaaggagacatcaaggaatgcggacaaggatatcgttaccaagcaatggcatgctacgatcaaaatggcaggcttgtggaaacatctagatgtaacagccatggttacattgaggaggcctgcatcatcccctgcccctcagactgcaagctcagtgagtggtccaactggtcgcgctgcagcaagtcctgtgggagtggtgtgaaggttcgttctaaatggctgcgtgaaaaaccatataatggaggaaggccttgccccaaactggaccatgtcaaccaggcacaggtgtatgaggttgtcccatgccacagtgactgcaaccagtacctatgggtcacagagccctggagcatctgcaaggtgacctttgtgaatatgcgggagaactgtggagagggcgtgcaaacccgaaaagtgagatgcatgcagaatacagcagatggcccttctgaacatgtagaggattacctctgtgacccagaagagatgcccctgggctctagagtgtgcaaattaccatgccctgaggactgtgtgatatctgaatggggtccatggacccaatgtgttttgccttgcaatcaaagcagtttccggcaaaggtcagctgatcccatcagacaaccagctgatgaaggaagatcttgccctaatgctgttgagaaagaaccctgtaacctgaacaaaaactgctaccactatgattataatgtaacagactggagtacatgtcagctgagtgagaaggcagtttgtggaaatggaataaaaacaaggatgttggattgtgttcgaagtgatggcaagtcagttgacctgaaatattgtgaagcgcttggcttggagaagaactggcagatgaacacgtcctgcatggtggaatgccctgtgaactgtcagctttctgattggtctccttggtcagaatgttctcaaacatgtggcctcacaggaaaaatgatccgaagacgaacagtgacccagccctttcaaggtgatggaagaccatgcccttccctgatggaccagtccaaaccctgcccagtgaagccttgttatcggtggcaatatggccagtggtctccatgccaagtgcaggaggcccagtgtggagaagggaccagaacaaggaacatttcttgtgtagtaagtgatgggtcagctgatgatttcagcaaagtggtggatgaggaattctgtgctgacattgaactcattatagatggtaataaaaatatggttctggaggaatcctgcagccagccttgcccaggtgactgttatttgaaggactggtcttcctggagcctgtgtcagctgacctgtgtgaatggtgaggatctaggctttggtggaatacaggtcagatccagaccggtgattatacaagaactagagaatcagcatctgtgcccagagcagatgttagaaacaaaatcatgttatgatggacagtgctatgaatataaatggatggccagtgcttggaagggctcttcccgaacagtgtggtgtcaaaggtcagatggtataaatgtaacagggggctgcttggtgatgagccagcctgatgccgacaggtcttgtaacccaccgtgtagtcaaccccactcgtactgtagcgagacaaaaacatgccattgtgaagaagggtacactgaagtcatgtcttctaacagcacccttgagcaatgcacacttatccccgtggtggtattacccaccatggaggacaaaagaggagatgtgaaaaccagtcgggctgtacatccaacccaaccctccagtaacccagcaggacggggaaggacctggtttctacagccatttgggccagatgggagactaaagacctgggtttacggtgtagcagctggggcatttgtgttactcatctttattgtctccatgatttatctagcttgcaaaaagccaaagaaaccccaaagaaggcaaaacaaccgactgaaacctttaaccttagcctatgatggagatgccgacatgtaacatataacttttcctggcaacaaccagtttcggctttctgacttcatagatgtccagaggccacaacaaatgtatccaaactgtgtggattaaaatatattttaatttttaaaaatggcatcataaagacaagagtgaaaatcatactgccactggagatatttaagacagtaccacttatatacagaccatcaaccgtgagaattataggagatttagctgaatacatgctgcattctgaaagttttatgtcatcttttctgaaatctaccgactgaaaaaccactttcatctctaaaaaataatggtggaattggccagttaggatgcctgatacaagaccgtctgcagtgttaatccataaaacttcctagcatgaagagtttctaccaagatctccacaatactatggtcaaattaacatgtgtactcagttgaatgacacacattatgtcagattatgtacttgctaataagcaattttaacaatgcataacaaataaactctaagctaagcagaaaatccactgaataaattcagcatcttggtggtcgatggtagattttattgacctgcatttcagagacaaagcctcttttttaagacttcttgtctctctccaaagtaagaatgctggacaagtactagtgtcttagaagaacgagtcctcaagttcagtattttatagtggtaattgtctggaaaactaatttacttgtgttaatacaatacgtttctactttccctgattttcaaactggttgcctgcatcttttttgctatatggaaggcacatttttgcactatattagtgcagcacgataggcgcttaaccagtattgccatagaaactgcctcttttcatgtgggatgaagacatctgtgccaagagtggcatgaagacatttgcaagttcttgtatcctgaagagagtaaagttcagtttggatggcagcaagatgaaatcagctattacacctgctgtacacacacttcctcatcactgcagccattgtgaaattgacaacatggcggtaatttaagtgttgaagtccctaaccccttaaccctctaaaaggtggattcctctagttggtttgtaattgttctttgaaggctgtttatgactagatttttatatttgttatctttgttaagaaaaaaaaagaaaaaggaactggatgtctttttaattttgagcagatggagaaaataaataatgtatcaatgacctttgtaactaaaggaaatcaaaattatatgttgatttttctttctctctgatttcccagtttcagattgaatgtctgtcttgcaggcagttatttcaaaatccatagtcttttgcctttctcactggcaaaatttgaagcaatctctctcttcaatgtatgatttcaaaactaaaattttaaaagagaagaataaatatcttatccaagacttcattgcatatctagagcaagagtggcaaactttttctgtaaaggactatatagaaaatattttccacaaggtctatgttgcaacttctcaactttgccattctgctaatgcaatcttagtaaataaaagaatgaacataactgttttcctatataacttcccttacaaaaacagccagtaggccacctttgcaggtcatattttgctgacccctggtctggatgttttttaatgctagctctccatggtgtcatctttcgttttcattcatttttcaaaggtgagaatttacagtcattacatttcccactgtatcagaagtagctgtattttctacagatgtagccctgggaatggcacagtggaacaatatttaactaactcagcagacttcttgcaaatgaaaagaaaactcaaaaggtgccattaaaaagcaataccattttcaggcaacattattgcaacaacctttgtgagagaggacactagcttttcaggatatttgaaaaataatataactaacatgctattttatcttaaacagctgtgttcctgtaaaagtgttttggattagaaggtatttttaccttcatactggagtatgaactccccctgctgttcattagagtatttgcaaagattacaaaagacatcaggttaggatgtcattgtccccaaaatgaaaatatttgtatgtatttcaataagaactaaatgcagtaagcgctaattcctcattcaatattcactatattaggaaatgtaaatcatttcaaagagaacaggtagtcaggaagatgaaaattttactttaaagcaaggatttaaacaaagaaaaccataaaaacaaagtccattttatctgttttattaaagaggaagaaaaaataatattgttactttaaaactgtgattgtcaatatatataatcaggggtcttccagataatcctaaagttgatctctttgaataaaataaactccatttgtccaaaatagttaagaaagactctattgtggttacttgaaaaattcagctaaccctcttatgaaatgtgtgcaacacctattaattttgaaatgtgggatagaatacctactaaagctatatttaaggtattgtaaaatgtcattaattcagaaaagattagtatcatcctacctataagaattaaattagcaaatcaaaattatagaatgttatgaaaaatacattttattcataaattaattagattttacagcatatatgcatgcatatatatatatacacacacacacacacacacccatatatatgagagagaggaaattaggttcaacatgacctaatattagagaacatattggaaggtcactgtttgttaggataaataaatactggataacctcgttagaaaggtggaaagaggagaacgtagcaatctggctattttggacaaagggaaggttattatattcatgtttataacatgaaaatggccttatttttgtttctgacatgagacaataatgagttaaatattttattggaatttcatcaaaaccttatttttactgttccttctaaaaatttgggaggggaatatagtagtaacttctctattgacacttttacctcaccgagggggtaaaagatgtagaactgctgttccctagaatgaaggtctgttgtttggtttttaaaaaaagaagagcagaaaaagtttgacacagtattatatgattttgctggactatttcactagaaaccacgtagaataggactaactgatctcttttgaaaggggctgatttgcttattcatcaataactaatccaatatgtggacagtaatcttaatttcaaagccttttttgaggatactacggtgtagcttaagtgaatttaaaaagagtgcataaaagcttgtttttaattacttataacaaagcacaaattacttctaacaaggcaaatatttattgacatataagacattctgctaggcattgtaaaaaaaaaaaaaaaagtaatggcttttgtatacataggtacttaagacagacatccaaaagaaggaaaaaatgcttaatgagtgaagatagcactgaaacatgttgacttttactctaggtttgtttttagatggggttagtattttcccctacgtaatgtacatgtctttaggccacagtattaggagtttgatttttccctgccccaccctcaggtaggtggcagtggtcattgtagcttaatcagacccctgttaagttcctgggcatttcaaggtaaattcacgtcttccttctcacagaaattgaatagacctctcacacacttatttacctcccccaacaacctgtatccttggccctttctgattttctctgcttggtctctttctactcttgacagctattcttacctacttcccactaaacatgcccaattttttaattaaaaaaaaaagaattcctatatttccttccctattagaatcaaagtgtcactcactcagagtgtgaacatcctgatttacaccaaatccactggtcaaagaaatgaataaagggaaaaggaaaaatgtggaaggcatgcaggaaaaaaaaaaaaaaaaaagcccttttctgtaaaaatgtccatgcctacacttgctttagaccatcccatatcctcctgcctccatctcgagtctttgttcacattccacaccacaagacagtgagggaggactgtacttttcagtaccatacagaagggagagcctgttctaaagctgacgtgggtctcaatagtacatgagaagcacaattcccaaatgatctgggatctcctgtttataaatatattaccaaattcaagtgtgggttcttcttctatttccactgtaagattgagggtgttgaaaaaacagtagtccttgagcaaatctgtgaggaatcttgtcagaatgtgatctttcctgggactagtttagttgtgagaactacatctgcttgttctgggaataagggtctccccaggtcctgccctcaggtggccctggctgcaggacaggatttcaggtatcagaaactggtgtgtgagccaagcaaatagttatccatttataaaaaggatggtttgcagtgtgttataataattttatatctagcagggacaatgaaaaatggttatgtctacattattaaatgaatgaaactaatgctaaacagttaattttgcatttttatttttctgtgtttctaccaagtcagatcttaaaaatgggcagtatatgaagagctttagtttgtgggagtcatgttttcagtgatcaaaaatctctatattatttagaatataattgtcagtcttgttgtgttttgtagatttattgtttttaagccatagtttctagattacctacttttaaccctcaagtaaacttttccctatttttcagaaatctgtttgtgtttatttttagaaagtaaacatttagattccttttctggtactttttttctccatttctatcacacagatttgaagagatcagtatctatcattgtaaaatataactttattaaaatttgcaatctgtctttctataaattattattccctaaatgtgtattttatgttaatttaactacctgaatatttattttgtttaaataaggaggcagctgtctaagaatttttgttaatattactttgtaaattgcgtaatgtaatttattttacattatgtacatttcctttttaacgcacaaaatgtctatgtacaattaactacagatttgcaaataatgtcactaagctttattcagtcaatacagatggcatgtgaagatgtaatatgcttttttacattttcatatgggttatttgtaaataactatattgctgacaaacataaatacggaaatcattttcattctaatcatgtgtatctgtgttttgatttgcaccaattgcttcttaaatatggattaaaatttttttccagtgaa

SEQ ID NO: 17 - protein, 73 aa, synthetic construct
ELYDWRLGPWNQCQPVISKSLEKPLECIKGEEGIQVREIACIQKDKDIPAEDIICEYFEPKPLLEQACLIPCQ

SEQ ID NO: 18 - protein, 145 aa, synthetic construct
AAQGEAEAPTLYLWKTGPWGRCMGDECGPGGIQTRAVWCAHVEGWTTLHTNCKQAERPNNQQNCFKVCDWHKELYDWRLGPWNQCQPVISKSLEKPLECIKGEEGIQVREIACIQKDKDIPAEDIICEYFEPKPLLEQACLIPCQ

SEQ ID NO: 19 - protein, 21 aa, Homo sapiens
MPMDLILVVWFCVCTARTVVG

SEQ ID NO: 20 - DNA, 3129 bp, Homo sapiens
  source 1..3129: mol_type = other DNA
ggggctgccttcccgggcgcatatgcgagcgcagcacccggcgctgccgagccacctcccccgccgcccgctagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccggaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagacattgatgaatgtgccttaagaactcacacctgttggaacgattctgcctgcatcaacctggcagggggctttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatgttgatgtattttttggtttattttgtgtactaacataatagagagagactcagctccttttatttattttgttgatttatggatcaaattctaaaataaagttgcctgttgtgacttttgtcccatctactgcatacttagtgctgagatccctgtaaaatgttttgatgaaaatatgtatgtagagtccagtcgcattatacatacatttcatagtgctgaaccttcttaaatgcctactcattcagcttaaacaggctgaagccaagtatgacaaagaggggaagggccaaaaacataatcaaagaataattttaaagagaattcttgtctctcttgcaaaaa

SEQ ID NO: 21 - protein, 25 aa, synthetic construct
MIASSVRHAVILLLVAVAMMAAVIA

SEQ ID NO: 22 - protein, 26 aa, unidentified
  source 1..26: note = Leishmania tarentolae
MIASSVRHAVILLLVAVAMMAAAVIA

SEQ ID NO: 23 - protein, 746 aa, Homo sapiens
MQAKKRYFILLSAGSCLALLFYFGGLQFRASRSHSRREEHSGRNGLHHPSPDHFWPRFPDALRPFVPWDQLENEDSSVHISPRQKRDANSSIYKGKKCRMESCFDFTLCKKNGFKVYVYPQQKGEKIAESYQNILAAIEGSRFYTSDPSQACLFVLSLDTLDRDQLSPQYVHNLRSKVQSLHLWNNGRNHLIFNLYSGTWPDYTEDVGFDIGQAMLAKASISTENFRPNFDVSIPLFSKDHPRTGGERGFLKFNTIPPLRKYMLVFKGKRYLTGIGSDTRNALYHVHNGEDVVLLTTCKHGKDWQKHKDSRCDRDNTEYEKYDYREMLHNATFCLVPRGRRLGSFRFLEALQAACVPVMLSNGWELPFSEVINWNQAAVIGDERLLLQIPSTIRSIHQDKILALRQQTQFLWEAYFSSVEKIVLTTLEIIQDRIFKHISRNSLIWNKHPGGLFVLPQYSSYLGDFPYYYANLGLKPPSKFTAVIHAVTPLVSQSQPVLKLLVAAAKSQYCAQIIVLWNCDKPLPAKHRWPATAVPVVVIEGESKVMSSRFLPYDNIITDAVLSLDEDTVLSTTEVDFAFTVWQSFPERIVGYPARSHFWDNSKERWGYTSKWTNDYSMVLTGAAIYHKYYHYLYSHYLPASLKNMVDQLANCEDILMNFLVSAVTKLPPIKVTQKKQYKETMMGQTSRASRWADPDHFAQRQSCMNTFASWFGYMPLIHSQMRLDPVLFKDQVSILRKKYRDIERL

SEQ ID NO: 24 - protein, 718 aa, Homo sapiens
MCASVKYNIRGPALIPRMKTKHRIYYITLFSIVLLGLIATGMFQFWPHSIESSNDWNVEKRSIRDVPVVRLPADSPIPERGDLSCRMHTCFDVYRCGFNPKNKIKVYIYALKKYVDDFGVSVSNTISREYNELLMAISDSDYYTDDINRACLFVPSIDVLNQNTLRIKETAQAMAQLSRWDRGTNHLLFNMLPGGPPDYNTALDVPRDRALLAGGGFSTWTYRQGYDVSIPVYSPLSAEVDLPEKGPGPRQYFLLSSQVGLHPEYREDLEALQVKHGESVLVLDKCTNLSEGVLSVRKRCHKHQVFDYPQVLQEATFCVVLRGARLGQAVLSDVLQAGCVPVVIADSYILPFSEVLDWKRASVVVPEEKMSDVYSILQSIPQRQIEEMQRQARWFWEAYFQSIKAIALATLQIINDRIYPYAAISYEEWNDPPAVKWGSVSNPLFLPLIPPQSQGFTAIVLTYDRVESLFRVITEVSKVPSLSKLLVVWNNQNKNPPEDSLWPKIRVPLKVVRTAENKLSNRFFPYDEIETEAVLAIDDDIIMLTSDELQFGYEVWREFPDRLVGYPGRLHLWDHEMNKWKYESEWTNEVSMVLTGAAFYHKYFNYLYTYKMPGDIKNWVDAHMNCEDIAMNFLVANVTGKAVIKVTPRKKFKCPECTAIDGLSLDQTHMVERSECINKFASVFGTMPLKVVEHRADPVLYKDDFPEKLKSFPNIGSL

SEQ ID NO: 25 - protein, 750 aa, Homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 26-36 - skipped (11 entries)

SEQ ID NO: 37 - protein, 15 aa, synthetic construct
GGGGANSTAPAPAPA

SEQ ID NO: 38 - protein, 504 aa, synthetic construct
MLLSPSLLLLLLLGAPRGCAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKA

SEQ ID NO: 39 - protein, 484 aa, synthetic construct
EGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKA

SEQ ID NO: 40 - protein, 10 aa, synthetic construct
HHHHHHHHHH

SEQ ID NO: 41 - protein, 530 aa, synthetic construct
MLLSPSLLLLLLLGAPRGCAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGGGGGANSTAPAPAPAHHHHHHHHHH

SEQ ID NO: 42 - protein, 118 aa, synthetic construct
YNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICK

SEQ ID NO: 43 - protein, 49 aa, synthetic construct
THGMPCMFPFQYNHQWHHECTREGREDDLLWCATTSRYERDEKWGFCPD

SEQ ID NO: 44 - protein, 20 aa, unidentified
  source 1..20: note = Leishmania major
MASRLVRVLAAAMLVAAAVS

SEQ ID NO: 45 - protein, 5 aa, synthetic construct
EAAAK

SEQ ID NO: 46 - protein, 5 aa, synthetic construct
  REGION >5: note = The entire sequence of amino acids 1-5 can be repeated one to twenty times.
GGGGS

(19) United States
(12) Patent Application Publication
(10) Pub. No.: US 2026/0217789 A1
(43) Pub. Date: Jul. 30, 2026

(54) GLYCOENGINEERED POLYPEPTIDES TARGETING ANTI-PODOCYTE AUTOANTIBODIES AND USES THEREOF

(71) Applicant: GlycoEra AG, Wädenswil (CH)

(72) Inventors: Ganesh Venkataraman Kaundinya, Bedford, MA (US); Tanmoy Chinmoy Ganguly, Westford, MA (US); Rainer Follador, Chur (CH); Manuela Mally, Watt (CH); Jonathan Albert Back, Villers-Le-Lac (FR); Michela Manni, Zürich (CH)

(21) Appl. No.: 19/144,979

(22) PCT Filed: Jan. 5, 2024
(86) PCT No.: PCT/IB2024/050116

§ 371 Date: Jul. 1, 2025

Related U.S. Application Data
(60) Provisional application No. 63/478,561, filed on Jan. 5, 2023.

(51) Int. Cl. C07K 14/705; A61K 38/00; C07K 16/28

(52) U.S. Cl. CPC C07K 14/7056; C07K 16/2851; A61K 38/00

(57) ABSTRACT

Provided herein are glycoengineered polypeptides comprising a first moiety comprising one or more peptides that specifically binds to an anti-podocyte autoantibody and a second moiety comprising one or more glycans conjugated to the first moiety at one or more glycosylation sites. Also provided herein are nucleic acid sequence encoding provided glycoengineered polypeptides. Further provided herein are compositions comprising glycoengineered polypeptides and/or nucleic acids encoding the same, as well as methods of making and using the same.

### CROSS REFERENCE TO RELATED APPLICATIONS

This application claims priority to and the benefit of U.S. Provisional Patent Application No. 63/478,561 filed on Jan. 5, 2023, the entire contents of which are hereby incorporated by reference in its entirety.

### BACKGROUND

Anti-podocyte autoantibodies are commonly found in autoimmune diseases such as membranous nephropathy, or “MN” (e.g., idiopathic membranous nephropathy, or “iMN”) and are often implicated in disease pathogenesis.

### SUMMARY

The present disclosure identifies certain challenges with existing therapies used to treat membranous nephropathy (e.g., idiopathic membranous nephropathy). For example, the present disclosure identifies limitations of some of the most widely used treatment options. Immunosuppressive therapies including steroids and cyclophosphamide are some of the most common therapies for treating membranous nephropathy (e.g., idiopathic membranous nephropathy). However, these therapeutic options do not directly act on anti-podocyte autoantibodies in circulation, tissue or organs; and do not reduce and/or deplete anti-podocyte autoantibodies or immune complexes comprising the same. Therefore, while the currently available treatment options may provide some relief to patients, these treatment options do not treat the underlying cause of disease.

The present disclosure also identifies limitations in certain non-specific therapies, such as B-cell directed therapies (e.g., Rituximab), which are used for treating membranous nephropathy (e.g., idiopathic membranous nephropathy). Such non-specific therapies, (e.g., non-specific B cell directed therapies) can lead to unwanted side effects such as organ damage or immunosuppression. Additionally, non-specific therapies (e.g., B-cell directed therapies) likely need to be dosed for a longer period of time to achieve a desired result, e.g., a clinical response. Furthermore, non-specific therapies (e.g., B-cell directed therapies) likely cannot produce rapid responses due to the lack of specificity and lack of targeting of disease-causing components, e.g., anti-podocyte autoantibodies or immune complexes comprising the same. Such non-specific therapies are also not able to disrupt and/or remove anti-podocyte autoantibodies or pathogenic immune complexes comprising the same.

Without wishing to be bound by any particular theory, the technologies provided in the present disclosure can address certain limitations identified in existing therapies used to treat diseases associated with anti-podocyte antibodies such as membranous nephropathy (e.g., idiopathic membranous nephropathy). Technologies provided herein specifically target autoantibodies or immune complexes comprising the same, and produce rapid, specific, and durable responses when administered to a subject. In some embodiments, technologies provided herein are anticipated to produce fewer and/or less severe unwanted side effects than non-specific therapies (e.g., B-cell directed therapies), due to the specificity in targeting immunogenic autoantibodies. In some embodiments, technologies provided herein can result in responses (e.g., clinical responses) in a shorter amount of time, as compared to non-specific therapies (e.g., B-cell directed therapies). In some embodiments, technologies provided herein do not need to be dosed for as long an extended period of time, e.g., as compared to non-specific therapies (e.g., B-cell directed therapies). In some embodiments, technologies provided herein can disrupt and/or remove immune complexes comprising autoantibodies disclosed herein. In some embodiments, technologies provided herein can result in improved depletion and/or removal of anti-podocyte autoantibodies or immune complexes comprising the same, as compared to non-specific therapies (e.g., B-cell directed therapies). Advantages associated with technologies disclosed herein can result in improved responses (e.g., clinical responses) in patients having or at risk of having membranous nephropathy (e.g., idiopathic membranous nephropathy).

Among other things, the present disclosure provides technologies for reducing and/or removing anti-podocyte autoantibodies by providing glycoengineered polypeptides that can simultaneously bind to a target autoantibody (e.g., an anti-podocyte autoantibody) and to an endocytic receptor via one or more glycans. Without wishing to be bound by theory, the present disclosure proposes that binding of a glycoengineered polypeptide disclosed herein to a target autoantibody and to an endocytic receptor induces internalization of the target autoantibody into a cell. In some embodiments, internalization of the target autoantibody results in degradation. The technologies disclosed herein also relate to nucleic acid molecules encoding glycoengineered polypeptides disclosed herein. Further provided herein are compositions comprising glycoengineered polypeptides disclosed herein or nucleic acid molecules encoding the same, and methods of making the same.

Technologies provided herein are particularly useful for reducing and/or removing anti-podocytes autoantibodies in subjects having, or at risk of having membranous nephropathy (e.g., idiopathic membranous nephropathy). Administration of compositions comprising glycoengineered polypeptides disclosed herein or nucleic acid molecules encoding the same can reduce and/or deplete anti-podocyte antibodies, thus treating the disease or ameliorating one or more symptoms of the disease.

Accordingly, the present disclosure provides a glycoengineered polypeptide comprising: (a) a first moiety comprising one or more peptides that specifically binds to an anti-podocyte autoantibody or a fragment or a complex thereof; and (b) a second moiety comprising one or more glycans conjugated to the first moiety at one or more glycosylation sites.

In some embodiments, an anti-podocyte autoantibody is an anti-PLA2R autoantibody or a fragment or a complex thereof. In some embodiments, an anti-PLA2R autoantibody binds to a PLA2R polypeptide, or a variant or fragment thereof.

In some embodiments, an anti-podocyte autoantibody is an anti-THSD7A autoantibody or a fragment or a complex thereof. In some embodiments, an anti-THSD7A autoantibody binds to a THSD7A polypeptide, or a variant or fragment thereof.

In some embodiments, an anti-podocyte autoantibody is an anti-NELL1 autoantibody or a fragment or a complex thereof. In some embodiments, an anti-NELL1 autoantibody binds to a NELL1 polypeptide, or a variant or fragment thereof.

In some embodiments, an anti-podocyte autoantibody is an anti-NEP autoantibody or a fragment or a complex thereof. In some embodiments, an anti-NEP autoantibody binds to a NEP polypeptide, or a variant or fragment thereof.

In some embodiments, an anti-podocyte autoantibody is an anti-EXT1 autoantibody or a fragment or a complex thereof. In some embodiments, an anti-EXT1 autoantibody binds to a EXT1 polypeptide, or a variant or fragment thereof.

In some embodiments, an anti-podocyte autoantibody is an anti-EXT2 autoantibody or a fragment or a complex thereof. In some embodiments, an anti-EXT2 autoantibody binds to a EXT2 polypeptide, or a variant or fragment thereof.

In some embodiments, a glycoengineered polypeptide is capable of binding to any one, or all, or a combination of: an anti-PLA2R autoantibody or a fragment or a complex thereof; an anti-THSD7A autoantibody or a fragment or a complex thereof; an anti-NELL autoantibody or a fragment or a complex thereof; an anti-NEP autoantibody or a fragment or a complex thereof; an anti-EXT1 autoantibody or a fragment or a complex thereof; and an anti-EXT2 autoantibody or a fragment or a complex thereof.

In some embodiments, a second moiety specifically binds to one or more endocytic receptors. In some embodiments, an endocytic receptor is or comprises an endocytic lectin. In some embodiments, an endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

In some embodiments, a glycan structure comprises a biantennary structure. In some embodiments, a glycan structure comprises a biantennary GalNAc. In some embodiments, a biantennary GalNac binds to an asialoglycoprotein receptor (ASGPR) or a fragment or variant thereof, or a complex comprising ASGPR.

Also provided herein is a composition comprising a glycoengineered polypeptide disclosed herein or a composition comprising a polynucleotide encoding a glycoengineered polypeptide disclosed herein.

In some embodiments, a composition comprises a glycoengineered polypeptide comprising a first moiety that specifically binds to an anti-PLA2R autoantibody or a fragment or a complex thereof.

In some embodiments, a composition comprises a glycoengineered polypeptide comprising a first moiety that specifically binds to an anti-THSD7A autoantibody or a fragment or a complex thereof.

In some embodiments, a composition comprises a glycoengineered polypeptide comprising a first moiety that specifically binds to an anti-NELL autoantibody or a fragment or a complex thereof.

In some embodiments, a composition comprises: (i) a first glycoengineered polypeptide comprising a first moiety that specifically binds to an anti-PLA2R autoantibody or a fragment or a complex thereof; (ii) a second glycoengineered polypeptide comprising a first moiety that specifically binds to an anti-THSD7A autoantibody or a fragment or a complex thereof; and/or (iii) a third glycoengineered polypeptide comprising a first moiety that specifically binds to an anti-NELL autoantibody or a fragment or a complex thereof.

The disclosure further provides a Leishmania host cell expressing a glycoengineered polypeptide disclosed herein.

Also provided herein is a method comprising: administering to a subject a pharmaceutical composition comprising a glycoengineered polypeptide disclosed herein or a polynucleotide encoding a glycoengineered polypeptide disclosed herein.

In some embodiments, a subject has or is diagnosed as having idiopathic membranous nephropathy (iMN).

Also provided herein is a method of treating and/or preventing idiopathic membranous nephropathy (iMN) in a subject, the method comprising, administering to a subject a pharmaceutical composition comprising a glycoengineered polypeptide disclosed herein or a polynucleotide encoding a glycoengineered polypeptide disclosed herein.

In some embodiments, a glycoengineered polypeptide is capable of simultaneously binding to an anti-podocyte autoantibody with the first moiety and binding to an endocytic receptor-expressing cell with the second moiety, thereby causing the anti-podocyte autoantibody to be internalized into a cell. In some embodiments, internalization comprises transporting to a lysosome and/or degradation. In some embodiments, an anti-podocyte autoantibody and the glycoengineered polypeptide are internalized.

In some embodiments, administration of the pharmaceutical composition reduces a level of an anti-podocyte autoantibody as compared to a subject who has not been administered the pharmaceutical composition or as compared to the same subject prior to administration of the pharmaceutical composition.

In some embodiments, administration of the pharmaceutical composition reduces and/or prevents formation and/or accumulation of an immune complex as compared to a subject who has not been administered the pharmaceutical composition or as compared to the same subject prior to administration of the pharmaceutical composition.

In some embodiments, an immune complex comprises one or more: IgG antibodies, the anti-podocyte autoantibody, an antigen recognized by the anti-podocyte autoantibody, complement components, or combinations thereof.

In some embodiments, an immune complex comprising one or more complement components comprises a membrane attack complex. In some embodiments, one or more complement components comprise: C3, C5b, C6, C7, C8, and/or C9.

In some embodiments, administration of the pharmaceutical composition reduces activation of a complement pathway or component thereof as compared to a subject who has not been administered the pharmaceutical composition or as compared to the same subject prior to administration of the pharmaceutical composition. In some embodiments, a complement pathway comprises: a lectin pathway, a classical pathway or an alternative pathway.

In some embodiments, albumin levels and/or edema as compared to a subject who has not been administered the pharmaceutical composition or as compared to the same subject prior to administration of the pharmaceutical composition.

In some embodiments, administration of the pharmaceutical composition reduces and/or prevents thickening of glomerular capillary walls as compared to a subject who has not been administered the pharmaceutical composition or as compared to the same subject prior to administration of the pharmaceutical composition.

In some embodiments, administration of the pharmaceutical composition reduces and/or prevents changes in structure and/or function of podocytes as compared to a subject who has not been administered the pharmaceutical composition or as compared to the same subject prior to administration of the pharmaceutical composition.

In some embodiments, administration of the pharmaceutical composition prevents apoptosis in podocytes.

In some embodiments, administration of the pharmaceutical composition prevents an increase in intracellular calcium in human podocytes.

In some embodiments, administration of the pharmaceutical composition prevents a decrease in cellular motility and/or adhesion of podocytes.

In some embodiments, administration of the pharmaceutical composition treats and/or prevents the disease.

In some embodiments, administration of the pharmaceutical composition alleviates one or more symptoms of the disease.

Further provided herein is a method comprising assessing a level of an anti-podocyte autoantibody in a sample from a subject, and administering a pharmaceutical composition disclosed herein, if the level of the anti-podocyte autoantibody is higher than a comparator.

In some embodiments, a comparator comprises a predetermined reference sample such as a sample obtained from an otherwise similar subject who does not have a disease or disorder, or a symptom of a disease or disorder.

In some embodiments, an anti-podocyte autoantibody comprises: an anti-PLA2R autoantibody or a fragment or a complex thereof; an anti-THSD7A autoantibody or a fragment or a complex thereof; an anti-NELL autoantibody or a fragment or a complex thereof, or a combination thereof.

In some embodiments, a level of anti-podocyte autoantibody is assessed using an ELISA.

In some embodiments, a disease or disorder is iMN.

In some embodiments, a method is a treatment method.

In some embodiments, a method is a prevention method.

### BRIEF DESCRIPTION OF DRAWINGS

FIGS. 1A-1C show the binding kinetics of an exemplary PLA2R glycoengineered polypeptide to a PLA2R antibody or to human ASGPR1. The PLA2R antibody clone 12-6-5, was covalently immobilized (FIG. 1A) or immobilized using the Fc tag (FIG. 1B). For binding to human ASGPR1, biotinylated recombinant human ASGPR1 was used (FIG. 1C). SPR binding data were collected in single cycle kinetic experiments using a Biacore 8K.

FIGS. 2A-2D depict blocking capability of an exemplary PLA2R glycoengineered polypeptide against autoantibodies in iMN patient sera. Sixteen serum samples from iMN patients and two serum samples from control patients (labeled VA-014 and Control) were incubated with the indicated concentration of the PLA2R glycoengineered polypeptide Autoantibodies not blocked by the PLA2R glycoengineered polypeptide were then evaluated by ELISA. FIG. 2A-2C are a series of graphs showing the PLA2R autoantibodies in each sample expressed as relative units/ml (RU/mL). The dotted line represents the ELISA limit of detection. FIG. 2D depicts the data in FIGS. 2A-2C expressed as percentage of depletion at 10 nM of the PLA2R glycoengineered polypeptide. VA014 and Control are two non-iMN patient controls. BLOQ: below lower limit of quantification.

FIGS. 3A-3B depict the depletion of anti-PLA2R autoantibodies in serum from iMN patients with an exemplary PLA2R glycoengineered polypeptide. Serum samples from eight iMN patients were incubated with magnetic beads pre-coated with the PLA2R glycoengineered polypeptide. For depletion, samples containing the magnetic beads were placed on a magnet, and flow-through was collected and analyzed for autoantibodies levels by ELISA. The graph in FIG. 3A. shows the autoantibody level expressed as relative units/ml (Ru/mL) in the indicated patient serum sample before and after depletion with the PLA2R glycoengineered polypeptide coated beads. For each patient the data is presented as a pair of bars with the left bar showing the undepleted autoantibody level, and the right bar showing the level after depletion with the PLA2R glycoengineered polypeptide. FIG. 3B shows the data in FIG. 3A. expressed as a percentage of autoantibody depletion obtained with the PLA2R glycoengineered polypeptide.

FIGS. 4A-4C depict the ability of complexes comprising an exemplary PLA2R glycoengineered polypeptide and an anti-PLA2R antibody to be internalized and degraded in hepatocytes. FIG. 4A is a schematic representation of the experimental protocol. The PLA2R glycoengineered polypeptide was pre-incubated with an anti-PLA2R antibody (clone 12-6-5) and allowed to form complexes. The complexes were added to HepG2 cells or HepG2 ASGPR knock-out cells for 4 hours for internalization. HepG2 cells were then washed and collected at 0, 1, 2, 6 and 24 hours post wash and analyzed for the PLA2R glycoengineered polypeptide and 12-6-5 internalization by western blot. HepG2 ASGPR knock-out cells were then washed and collected at 0 hours post wash only and analyzed for the PLA2R glycoengineered polypeptide and 12-6-5 internalization by western blot. FIG. 4B is a blot depicting the PLA2R glycoengineered polypeptide (referred to as “PLA2R-glyco-polypep in FIGS. 4B and 4C”) protein levels using an anti-His antibody. FIG. 4C is a blot depicting an anti-PLA2R antibody (clone 12-6-5) protein levels using an anti-mouse IgG antibody. HC: Heavy chain. LC: Light chain. KO: knock-out.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

PLA2R: The term “PLA2R” is used herein in reference to M-type phospholipase A2 receptor polypeptides as understood in the art. PLA2R is a transmembrane glycoprotein and belongs to the mannose receptor family. PLA2R is also known as the M-type receptor, or C-type lectin domain family 13 member 3. PLA2R protein is encoded by the PLA2R gene. PLA2R protein is cleaved into a soluble secretory form: soluble PLA2R. Amino acid sequences for full-length PLA2R and/or for nucleic acids that encode it can be found in a public database such as GenBank, UniProt and Swiss-Prot. For example, an amino acid sequence of human PLA2R (SEQ ID NO: 1, for which residues 1-20 represent the signal peptide, residues 21-397 represent the extracellular region, residues 1398-1418 represent the helical domain, and residues 1419-1463 represent the cytoplasmic domain can be found as UniProt/Swiss-Prot Accession No. Q13018; a nucleic acid sequence (SEQ ID NO: 13) encoding human PLA2R can be found at GenBank: U17033.1 Those skilled in the art will appreciate that sequences presented in SEQ ID NOs: 1 and 13 are exemplary, and certain variations (including, for example, conservative substitutions in SEQ ID NO:1, codon-optimized variants of SEQ ID NO:13, etc) are understood to also be or encode human PLA2R; additionally, those skilled in the art will appreciate that homologs and orthologs of human PLA2R are known and/or knowable through the exercise of ordinary skill and which may be useful in the present invention, for example, based on degree of sequence identity, presence of one or more characteristic sequence elements, and/or one or more shared activities.

THSD7A: The term “THSD7A” is used herein in reference to Thrombospondin type-1 domain-containing protein 7A polypeptides as understood in the art. THSD7A is a type 1 transmembrane protein and is also known as KIAA0960. THSD7A protein is cleaved into a soluble secretory form: soluble THSD7A. THSD7A protein is encoded by the THSD7A gene. Amino acid sequences for full-length THSD7A and/or for nucleic acids that encode it can be found in a public database such as GenBank, UniProt and Swiss-Prot. For example, an amino acid sequence of human THSD7A (SEQ ID NO: 10, for which residues 1-47 represent the signal peptide, can be found as UniProt/Swiss-Prot Accession No. Q9UPZ6; a nucleic acid sequence (SEQ ID NO: 17) encoding human THSD7A can be found at NM Accession No.: NM_015204.3. Those skilled in the art will appreciate that sequences presented in SEQ ID NOs: 10 and 17 are exemplary, and certain variations (including, for example, conservative substitutions in SEQ ID NO:10, codon-optimized variants of SEQ ID NO:17, etc) are understood to also be or encode human THSD7A; additionally, those skilled in the art will appreciate that homologs and orthologs of human THSD7A are known and/or knowable through the exercise of ordinary skill and which may be useful in the present invention, for example, based on degree of sequence identity, presence of one or more characteristic sequence elements, and/or one or more shared activities.

NELL1: The term “NELL1” or “NELL-1” is used herein in reference to Protein kinase C-binding protein NELL1 polypeptides as understood in the art. NELL1 is a protein kinase and is also known as NRP1. NELL1 protein is encoded by the NELL1 gene. Amino acid sequences for full-length NELL1 and/or for nucleic acids that encode it can be found in a public database such as GenBank, UniProt and Swiss-Prot. For example, an amino acid sequence of human NELL1 (SEQ ID NO: 9, for which residues 1-21 represent the signal peptide, can be found as UniProt/Swiss-Prot Accession No. Q92832; a nucleic acid sequence (SEQ ID NO: 20) encoding human NELL1 can be found at NM Accession No.: NM_201551.2. Those skilled in the art will appreciate that sequences presented in SEQ ID NOs: 9 and 20 are exemplary, and certain variations (including, for example, conservative substitutions in SEQ ID NO:9, codon-optimized variants of SEQ ID NO:20, etc) are understood to also be or encode human NELL1; additionally, those skilled in the art will appreciate that homologs and orthologs of human NELL1 are known and/or knowable through the exercise of ordinary skill and which may be useful in the present invention, for example, based on degree of sequence identity, presence of one or more characteristic sequence elements, and/or one or more shared activities.

EXT1 and EXT2: The terms “EXT1” or EXT-1, and “EXT2” or “EXT-2” are used herein in reference to exostosin peptides exostosin-1 and exostosin-2, respectively, as understood in the art. EXT1 and EXT2 have been identified as the target antigens in some cases of iMN, including in particular double-negative MN, in which neither autobodies to PLA2 nor THSD7A have been identified. Sethi et al. (2019) J. Am. Soc. Nephrol. 30:1123-1136; Ronco et al. (2021) Nature Rev Disease Primers 7:69. The amino acid sequences for full-length EXT1 and/or EXT2, as well as for nucleic acids that encode them can be found in a public database such as GenBank, UniProt and Swiss-Prot. For example, an amino acid sequence of human EXT1 (SEQ ID NO: 23) can be found as UniProt/Swiss-Prot Accession No. Q16394; and an amino acid sequence of human EXT2 (SEQ ID NO: 24) can be found as UniProt/Swiss-Prot Accession No. Q93063. Those skilled in the art will appreciate that the sequence presented in SEQ ID NO: 23 and 24 are exemplary, and certain variations (including, for example, conservative substitutions, codon-optimized variants) are understood to also be or encode human EXT1 or human EXT2, respectively. Additionally, those skilled in the art will appreciate that homologs and orthologs of human EXT1 and EXT2 are known and/or knowable through the exercise of ordinary skill and which may be useful in the present invention, for example, based on degree of sequence identity, presence of one or more characteristic sequence elements, and/or one or more shared activities.

NEP: The term “NEP” is used herein in reference to neutral endopeptidase or neprilysin polypeptides as understood in the art. NEP has been identified as the podocyte target antigen of circulating antibodies in cases of antenatal and neonatal membranous glomerulo-nephritis (“MGN”), a type of MN. Debiac et al., (2004) Lancet 364:1252-1259; Debiac et al. (2002) N Engl J Med 346:2053-2060. NEP protein is encoded by the membrane metalloendopeptidase or “MME” gene. Amino acid sequences for full-length NEP and/or for nucleic acids that encode it can be found in a public database such as GenBank, UniProt and Swiss-Prot. For example, an amino acid sequence of human NEP (SEQ ID NO: 25) can be found as UniProt/Swiss-Prot Accession No. P08473; a nucleic acid sequence encoding human NEP can be found in GenBank at NM Accession No.: NM_000902.5. Those skilled in the art will appreciate that sequences presented in SEQ ID NOs: 25 are exemplary, and certain variations (including, for example, conservative substitutions; codon-optimized variants) are understood to also be or encode human NEP; additionally, those skilled in the art will appreciate that homologs and orthologs of human NEP are known and/or knowable through the exercise of ordinary skill and which may be useful in the present invention, for example, based on degree of sequence identity, presence of one or more characteristic sequence elements, and/or one or more shared activities.

Glycans: As used herein, the term “glycan” refers to one or more saccharides or sugar chains that can be attached to a protein or lipid to form a glycoconjugate. A glycan conjugated to a protein forms a glycoprotein. A glycan conjugated to a nitrogen atom of an amino acid residue is an N-linked glycan and a glycan conjugated to an oxygen atom of an amino acid residue is an O-linked glycan. As will be appreciated by those of ordinary skill in the art, the structure of a glycan indicates if a specific glycan is an N-linked glycan.

Glycoengineered: As used herein, the term “glycoengineered,” or an equivalent thereof means a process of glycosylating a target protein (e.g., a glycoengineered polypeptide disclosed herein), or a target protein made by such process. In some embodiments, the process uses a host cell system that has one or more enzymes (e.g., pathways) that provides for glycosylation of the target protein; in some other embodiments, the process is performed by chemically attaching one or more glycans to a target protein, e.g., using Click chemistry. Such a host cell system can be genetically engineered to introduce a glycosylation pathway to selectively glycosylate a target protein with a particular glycan structure. A host cell used to generate a glycoengineered target protein can include, for example, a recombinant nucleic acid encoding a target protein; and a recombinant nucleic acid encoding a heterologous glycosyltransferase. The host cell system used for glycoengineering (e.g, to generate a glycoengineered protein) can introduce N-linked glycosylation. The host cell used for glycoengineering or to generate a glycoengineered target protein can be a mammalian cell, an insect cell, a yeast cell, a bacterial cell, a plant cell, a microalgae, or a protozoa. The protozoa used for glycoengineering can be a species of Leishmania. A glycoengineered target protein also includes a target protein that has been engineered to be selectively glycosylated at one or more specific sites when generated in the host cell system.

Glycoengineered polypeptide: As used herein, a “glycoengineered polypeptide” is a polypeptide that mediates the internalization and/or degradation of a target protein by specifically binding to a target protein (e.g., an anti-podocyte autoantibody) and engaging with one or more endocytic receptors. In some embodiments, binding (e.g., simultaneous binding) of a glycoengineered polypeptide to a target protein and an endocytic receptor internalizes a target protein and/or activates one or more degradation pathways.

Glycosylation site: As used herein, the term “glycosylation site” refers to a site of glycosylation in a protein. Such a glycosylation site, also referred to as a glycosite herein, can be naturally present in the amino acid sequence of a protein or recombinantly engineered into the protein by addition or substitution or deletion of amino acids. In some embodiments, a glycosylation site is present in a so-called glycotag that is fused to a glycoengineered polypeptide disclosed herein. In certain embodiments, a glycotag is fused to a protein to create a bispecific binding protein. As used herein a glycotag refers to a peptide containing consensus N-glycosylation site sequence fused to N- or a C-terminal or both termini of a protein or polypeptide. In some embodiments, the glycotag is fused to the C-terminus of the of the glycoengineered polypeptide disclosed herein via a peptide linker. In some embodiments, the glycotag is fused to the N-terminus of the glycoengineered polypeptide disclosed herein via a peptide linker. In some embodiments, the peptide linker is a consensus peptide sequence. In some embodiments, the consensus peptide sequence is 1, 2, 3, 4, 5, 6, 7 or more amino acid residues in length. In some embodiments, the bifunctional protein provided herein contains 1, 2, 3, 4, 5, 6, 7, 8, 9, 10 or more glycotags.

Endocytic receptor: As used herein, the term “endocytic receptor” refers to a receptor or a fragment thereof that binds to a target and internalizes the target into a cell. In some embodiments, an endocytic receptor recognizes and binds to one or more glycans on a target. In some embodiments, binding of an endocytic receptor to a target internalizes the target into a cell, e.g., into a lysosome or phagosome. In some embodiments, an endocytic receptor is or comprises an endocytic lectin. In some embodiments, an endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

Anti-podocyte autoantibody As used herein, the term “anti-podocyte autoantibody” refers to an antibody produced in an organism (e.g., a mammal) which specifically binds to an epitope on an antigen endogenous to a podocyte (e.g., a podocyte autoantigen). A podocyte autoantigen is expressed by a podocyte (e.g., present inside a podocyte and/or on the surface of a podocyte) and is encoded by a nucleic acid sequence naturally occurring in a podocyte genome. In some embodiments, a podocyte autoantigen comprises a PLA2R polypeptide or a variant or fragment thereof, a THSD7A polypeptide or a variant or fragment thereof, a NELL1 polypeptide or a variant or fragment thereof, a EXT1 polypeptide or a variant or fragment thereof, a EXT2 polypeptide or a variant or fragment thereof, a NEP polypeptide or a variant or fragment thereof, or combinations thereof. In some embodiments, an anti-podocyte autoantibody is or comprises a full antibody, an antigen binding fragment of an antibody, or a complex thereof. In some embodiments, an anti-podocyte autoantibody is or comprises an anti-PLA2R autoantibody, or a fragment or complex thereof. In some embodiments, an anti-podocyte autoantibody is or comprises an anti-THSD7A autoantibody, or a fragment or complex thereof. In some embodiments, an anti-podocyte autoantibody is or comprises an anti-NELL1 autoantibody, or a fragment or complex thereof. In some embodiments, an anti-podocyte autoantibody is or comprises an anti-EXT1 autoantibody, or a fragment or complex thereof. In some embodiments, an anti-podocyte autoantibody is or comprises an anti-EXT2 autoantibody, or a fragment or complex thereof. Other podocyte antigens have recently been discovered, which are potential autoantigens related to rare forms of MN and may be susceptible to treatment in accordance with the present invention. These include neutral endopeptidase (“nephralysin” or “NEP”), semaphorin 3B (“SEMA3B”), neural cell adhesion molecule 1 (“NCAM1”), protocadherin 7 (“PCDH7”) and serine protease HTRA1. Ronco et al. (2021) Nature Rev Disease Primers 7:69.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system, for example to achieve delivery of an agent that is, or is included in or otherwise delivered by, the composition. Those of ordinary skill in the art will be aware of a variety of routes that may, in appropriate circumstances, be utilized for administration to a subject, for example an animal or a human. In some embodiments, an animal is a domestic animal, such as a companion animal, e.g., a dog or a cat; in some embodiments, an animal is an animal used in agriculture (e.g., farming [e.g., a cow, a sheep or a horse]) or for recreation. For example, in some embodiments, administration may be systemic or local. Those skilled in the art will be aware of appropriate administration routes for use with particular therapies described herein, for example which include bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be by injection (e.g., intramuscular, intravenous, or subcutaneous injection). In some embodiments, injection may involve bolus injection, drip, perfusion, or infusion. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, an antibody agent can be formulated for oral delivery. For example, one with skill in the art will understand that an antibody agent disclosed herein can be formulated for oral delivery using technologies developed by Oramed (https://www.oramed.com/) or Premas (https://www.premasbiotech.com/).

Adult: As used herein, the term “adult” refers to a human eighteen years of age or older. In some embodiments, a human adult has a weight within the range of about 90 pounds to about 250 pounds.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments of a glycoengineered polypeptide disclosed herein, a first moiety comprising one or more peptides that specifically bind to a target autoantibody (e.g., an anti-podocyte autoantibody), has a high affinity to an autoantigen (e.g., a podocyte autoantigen). In some embodiments, a high affinity is an affinity of about 100-1000 pM. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference” ]. In some embodiments, affinity may be assessed relative to a contemporaneous reference; in some embodiments, affinity may be assessed relative to a historical reference. Typically, when affinity is assessed relative to a reference, it is assessed under comparable conditions.

Avidity: As is known in the art, “avidity” is a measure of the accumulated strength of multiple non-covalent interactions between two or more binding partners in a complex. Those skilled in the art are aware of a variety of assays that can be used to assess avidity, and will furthermore be aware of appropriate controls for such assays. In some embodiments, avidity can be determined by (1) a binding affinity of two or more binding partners in a complex; (2) valency of each of the binding partners in a complex; and/or (3) structural arrangements of two or more binding partners in a complex. In some embodiments, the avidity of binding between two or more binding partners is more than a sum of each binding affinity between the two or more binding partners. In some embodiments, avidity is also referred to as apparent affinity or functional affinity. In some embodiments of a glycoengineered polypeptide disclosed herein, a second moiety comprising one or more glycans which can bind to a receptor (e.g., an endocytic receptor) contributes to binding avidity of the glycoengineered polypeptide. In some embodiments, an endocytic receptor is ASGPR or a fragment or variant thereof. In some embodiments, avidity is assessed in a quantitative assay. In some embodiments, avidity is assessed over a plurality of concentrations. In some embodiments, avidity is assessed in the presence of one or more potential competitor entities (e.g., that might be present in a relevant—e.g., physiological—setting). In some embodiments, avidity may be assessed relative to a contemporaneous reference; in some embodiments, avidity may be assessed relative to a historical reference. Typically, when avidity is assessed relative to a reference, it is assessed under comparable conditions affinity is assessed relative to a reference, it is assessed under comparable conditions.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: as used herein refers to a member of the animal kingdom. In some embodiments, “animal” refers to humans; unless otherwise specified, in many embodiments, a human may be of either gender and/or at any stage of development. In some embodiments, “animal” refers to non-human animals; unless otherwise specified, in many embodiments, a non-human animal may be of any gender and/or at any stage of development. In certain embodiments, a non-human animal is a mammal (e.g., a rodent, a mouse, a rat, a rabbit, a monkey, a dog, a cat, a sheep, cattle, a primate, and/or a pig). In some embodiments, an animal may be, for example, a mammals, a bird, a reptile, an amphibian, a fish, an insect, a worm, etc. In some embodiments, an animal may be a transgenic animal, genetically engineered animal, and/or a clone.

Antibody: As used herein, the term “antibody” refers to a polypeptide that includes canonical immunoglobulin sequence elements sufficient to confer specific binding to a particular target antigen. In the case of an autoimmune disease, the antigen to which a pathogenic autoantibody binds is also referred to as an “autoantigen.” As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. In some embodiments, antibodies produced and/or utilized in accordance with the present disclosure include one or more modifications on an Fc domain, e.g., an effector null mutation, e.g., a LALA, LAGA, FEGG, AAGG, or AAGA mutation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of dog, cat, mouse, rabbit, primate, or human antibodies. In some embodiments, antibody sequence elements are human, humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., VHH [e.g., a camelid VHH] or NAR) alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, trifunctional antibodies, diabodies, and minibodies. etc. In some embodiments, relevant formats may be or include: Adnectins®; Affibodies®; Affilins®; Anticalins®; Avimers®; BiTE®s; cameloid antibodies; Centyrins®; ankyrin repeat proteins or DARPINs®; dual-affinity re-targeting (DART) agents; Fynomers®; shark single domain antibodies such as IgNAR; immune mobilixing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody format is or comprises a VHH, e.g., a camelid VHH. In some embodiments, a VHH is a multivalent VHH, e.g., a bivalent VHH. In some embodiments, an antibody comprises a single domain antibody, e.g., comprising one or more additional domains such as an Fc, a half-Fc (e.g., comprising an interchain cysteine mutant), an albumin domain, or combinations thereof. In some embodiments, an antibody comprises a single chain Fv, e.g., comprising one or more additional domains such as an Fc, a half-Fc (e.g., comprising an interchain cysteine mutant), an albumin domain, or combinations thereof. In some embodiments, an antibody comprises a polypeptide-Fc fusion. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (i.e., by expression of an engineered nucleic acid sequence. In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed subject matter.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, alpha-helix character, alpha-sheet character, coiled-coil character, random coil character, etc.), and/or by a particular functional feature (e.g., binding activity, enzymatic activity, folding activity, signaling activity, etc.).

Conjugate: The term “conjugate” as used herein refers to linking of one moiety to another moiety by in vitro methods (e.g., chemical synthesis) or in vivo (e.g., in a cell). In some embodiments, a moiety comprising one or more glycans (e.g., a second moiety) is conjugated to a different moiety, for example, at one or more glycosylation sites in vivo in a cell. In some embodiments, a moiety comprising one or more glycans (e.g., a second moiety) is conjugated to a different moiety, for example, at one or more glycosylation sites by chemical conjugation.

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Human: In some embodiments, a human is an embryo, a fetus, an infant, a child, a teenager, an adult, or a senior citizen.

Humanized: as is known in the art, the term “humanized” is commonly used to refer to antibodies (or antibody components) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc, which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having not more than a small number (e.g., 3, 2, or 1) an amino acid sequence changes relative to that of a reference CDR. In some embodiments, a CDR that is substantially identical to a reference CDR differs from that reference CDR by one or more amino acid changes at the end of the reference CDR; in some such embodiments, the relevant CDR is identical to the reference CDR other than at one or both ends. As is known in the art, CDR elements typically have a length within a range of a few amino acids (e.g., 3, 4, 5, 6, or 7) to about 20 or 30 amino acids (see, for example, Collis et al. J. Mol. Biol. 325:337, 2003, incorporated herein by reference); thus, in some embodiments, a CDR may be considered to be substantially identical to a reference CDR when it shares at least about 80% (or less for a shorter CDR), at least about 85%, at least about 90%, at least about 95%, at least about 98%, at least about 99%, or at least about 100% identity with the reference CDR.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Treat: As used herein, the term “treat,” “treatment,” or “treating” is used to refer to one or more of partial or complete alleviation, amelioration, relief, inhibition, prevention, delay of onset of, reduction in severity of and/or reduction in frequency (e.g., incidence) of one or more symptoms or features of a disease, disorder, and/or condition. In some embodiments, treatment may be prophylactic; for example may be administered to a subject who does not exhibit signs of a disease, disorder, and/or condition. In some embodiments, treatment may be administered to a subject who exhibits early signs of the disease, disorder, and/or condition, and may, for example, decrease risk of developing pathology associated with the disease, disorder, and/or condition and/or delay onset and/or decrease rate of development or worsening of one or more features of a disease, disorder and/or condition.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors, e.g., that are statistically correlated with increased risk of development of the relevant disease, disorder, and/or condition. Thus, in some embodiments, treatment may be prophylactic; in some embodiments, treatment may be therapeutic.

Variant: The term “variant”, as used herein, refers to a molecule or entity (e.g., that are or comprise a nucleic acid, protein, or small molecule) that shows significant structural identity with a reference molecule or entity but differs structurally from the reference molecule or entity, e.g., in the presence or absence or in the level of one or more chemical moieties as compared to the reference molecule or entity. In some embodiments, a variant also differs functionally from its reference molecule or entity. In many embodiments, whether a particular molecule or entity is properly considered to be a “variant” of a reference is based on its degree of structural identity with the reference molecule. As will be appreciated by those skilled in the art, a biological or chemical reference molecule in typically characterized by certain characteristic structural elements. A variant, by definition, is a distinct molecule or entity that shares one or more such characteristic structural elements but differs in at least one aspect from the reference molecule or entity. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

Disclosed herein are glycoengineered polypeptides and compositions comprising the same having the ability to degrade one or more anti-podocyte autoantibodies by binding to anti-podocyte autoantibodies with a first moiety and binding to an endocytic receptor with a second moiety comprising one or more glycans, thus targeting the anti-podocyte autoantibody for degradation. As exemplified herein, a glycoengineered polypeptide is engineered by introduction of glycosylation sites on a glycoengineered polypeptide, resulting in an engineered glycosylation profile that mediates endocytic receptor degradation of the glycoengineered polypeptides and the target to which it binds.

By customizing the N-glycosylation, a glycoengineered polypeptide described herein: 1) has homogeneous glycosylation; 2) can degrade large targets such as immune complexes; 3) has a defined ligand-to-antibody ratio; 4) has defined glycosylation sites; 6) can activate more diverse and powerful degradation receptors; and/or 6) can engage in protein degradation in a highly optimized manner. A glycoengineered polypeptide may be employed as a novel therapeutic to treat autoimmune diseases, e.g., a disease associated with anti-podocyte autoantibodies such as membranous nephropathy.

### Membranous Nephropathy

Membranous nephropathy (MN) is a glomerular disease that can occur at all ages. In adults, it is the most frequent cause of nephrotic syndrome. Membranous nephropathy is an autoimmune disease which is characterized by a thickening of glomerular capillary walls due to immune complex deposition on the subepithelial side of the glomerular basement membrane (GBM). Immune deposits typically comprise of immunoglobulin G (IgG) (e.g., one or more anti-podocyte autoantibodies), one or more podocyte autoantigens, and/or one or more complement complexes (e.g., a membrane attack complex [MAC]) (Ronco et al., (2021) “Membranous Nephropathy” Nature Reviews: Disease Primer 7:69). The complement MAC C5b-9 induces a variety of downstream pathways which can result in podocyte injury. Among the pathways which can be induced by the complement MAC include: protein kinases, lipid metabolism, reactive oxygen species, growth factors, gene transcription, endoplasmic reticulum stress and the ubiquitin-proteasome system (Ke et al., (2022) BMC Nephrology 23:313).

Depending on the cause, membranous nephropathy can be classified as primary (idiopathic) or secondary membranous nephropathy, which account for 75%-80% and 20%-25% of MN, respectively. Primary or idiopathic membranous nephropathy cases are most common and are often associated with autoantibodies recognizing podocyte autoantigens which can form immune complexes along the glomerular basement membrane (GBM). Secondary membranous nephropathy cases are often associated with autoimmune diseases, malignancies, infections, and/or drugs. A recent report has suggested a role for VEGFA in the pathogenesis of idiopathic membranous nephropathy. According to Ke et al. 2022, VEGFA induced activation of the PI3K-Akt signaling pathway can lead to vascular hyperpermeability resulting in increased filtration of inflammatory factors, complements, and cytokines.

Available treatment options for patients with membranous nephropathy, e.g., idiopathic membranous nephropathy, include administration of a VEGF pathway inhibitor, an anti-BAFF antibody, an anti-CD20 antibody, an anti-CD38 antibody, an anti-CD19 antibody, a Janus kinase inhibitor, a SYK inhibitor, a Factor B complement inhibitor, or a combination thereof. However, none of the available treatment options reduce and/or remove anti-podocyte autoantibodies or immune complexes comprising the same.

In some embodiments, one or more additional treatments (e.g., one or more current treatments listed above) may be administered prior to, substantially simultaneously with, or subsequent to one or more provided glycoengineered polypeptides.

### Anti-Podocyte Autoantibodies and Podocyte Autoantigens

Membranous nephropathy (e.g., idiopathic membranous nephropathy) may be induced by autoimmunity to one or more autoantigens expressed on podocytes (e.g., by the development of anti-podocyte autoantibodies). In some embodiments, anti-podocyte autoantibodies or immune complexes comprising the same form deposits at the glomerular basement membrane (GBM). In some embodiments, anti-podocyte autoantibodies or immune complexes comprising the same may cause podocyte injury. In some embodiments, anti-podocyte autoantibodies or immune complexes comprising the same may cause thickening of the GBM. In some embodiments, anti-podocyte autoantibodies or immune complexes comprising the same contribute to and/or result in Membranous nephropathy (e.g., idiopathic membranous nephropathy).

In some embodiments, an anti-podocyte autoantibody is an IgG antibody. In some embodiments, an anti-podocyte autoantibody is an IgA antibody. In some embodiments, an anti-podocyte autoantibody is an IgM antibody. In some embodiments, an anti-podocyte autoantibody is an IgD antibody. In some embodiments, an anti-podocyte autoantibody is an IgE antibody.

In some embodiments, an anti-podocyte autoantibody is an IgG4 antibody. In some embodiments, an anti-podocyte autoantibody does not directly induce complement activation (e.g., complement activation is independent of Fc receptor function). Without wishing to be bound by any particular theory, in some embodiments, where an anti-podocyte autoantibody is an IgG4 autoantibody, complement activation is a result of the formation of immune complexes comprising anti-podocyte antibodies and podocyte autoantigens.

Podocytes are cells in Bowman's capsule in the kidneys that wrap around capillaries of the glomerulus. Podocytes make up the epithelial lining of Bowman's capsule, the third layer through which filtration of blood takes place. Podocytes express a number of polypeptides (e.g., autoantigens) including but not limited to: phospholipase A2 receptor (PLA2R), Thrombospondin Type-1 Domain-Containing 7A (THSD7A), neutral endopeptidase (NEP), Neural Epidermal Growth Factor like 1 Protein (NELL-1), Exostosin 1 (EXT1), Exostosin 2 (EXT2), Semaphorin 3B (SEMA3B; UniProt/Swiss-Prot Accession No. Q13214), Neural Cell Adhesion Molecule 1 (NCAM1; UniProt/Swiss-Prot Accession No. P13591), and Protocadherin 7 (PCDH7; UniProt/Swiss-Prot Accession No. 060245) (Ronco et al. 2021).

In some embodiments, a podocyte autoantigen is expressed in a podocyte, e.g., in the cytoplasm, nucleus, peri-nucleus, or in a compartment in a cell. In some embodiments, a podocyte autoantigen is expressed on the cell surface of podocytes. In some embodiments, diseases associated with anti-podocyte autoantibodies such as membranous nephropathy (e.g., idiopathic membranous nephropathy) are associated with increased and/or aberrant expression of one or more podocyte autoantigens. In some embodiments, membranous nephropathy (e.g., idiopathic membranous nephropathy) is associated with increased and/or aberrant expression of one or more anti-podocyte autoantibodies or immune complexes comprising the same.

In some embodiments, a podocyte autoantigen comprises: PLA2R, THSD7A, NEP, NELL1, EXT1, EXT2, SEMA3B, NCAM1, PCDH7, or a combination thereof.

In some embodiments, a podocyte autoantigen is a PLA2R polypeptide, or a variant or fragment thereof.

In some embodiments, a podocyte autoantigen is a THSD7A polypeptide, or a variant or fragment thereof.

In some embodiments, a podocyte autoantigen is a NELL1 polypeptide, or a variant or fragment thereof.

In some embodiments, a podocyte autoantigen is a NEP polypeptide, or a variant or fragment thereof.

In some embodiments, a podocyte autoantigen is a EXT1 polypeptide, or a variant or fragment thereof.

In some embodiments, a podocyte autoantigen is a EXT2 polypeptide, or a variant or fragment thereof.

In some embodiments, a podocyte autoantigen is a SEMA3B polypeptide, or a variant or fragment thereof.

In some embodiments, a podocyte autoantigen is a NCAM1 polypeptide, or a variant or fragment thereof.

In some embodiments, a podocyte autoantigen is a PCDH7 polypeptide, or a variant or fragment thereof.

### Glycoengineered Polypeptides

Disclosed herein are glycoengineered polypeptides comprising: (a) a first moiety comprising one or more peptides that specifically binds to an anti-podocyte autoantibody or a fragment or a complex thereof; and (b) a second moiety comprising one or more glycans conjugated to the first moiety at one or more glycosylation sites.

In some embodiments, an anti-podocyte autoantibody is an anti-PLA2R autoantibody or a fragment or a complex thereof. In some embodiments, an anti-PLA2R autoantibody binds to PLA2R, or a variant or fragment thereof.

In some embodiments, an anti-podocyte autoantibody is an anti-THSD7A autoantibody or a fragment or a complex thereof. In some embodiments, an anti-THSD7A autoantibody binds to THSD7A, or a variant or fragment thereof.

In some embodiments, an anti-podocyte autoantibody is an anti-NELL1 autoantibody or a fragment or a complex thereof. In some embodiments, an anti-NELL1 autoantibody binds to NELL1, or a variant or fragment thereof.

In some embodiments, an anti-podocyte autoantibody is an anti-EXT1 autoantibody or a fragment or a complex thereof. In some embodiments, an anti-EXT1 autoantibody binds to EXT1, or a variant or fragment thereof.

In some embodiments, an anti-podocyte autoantibody is an anti-EXT2 autoantibody or a fragment or a complex thereof. In some embodiments, an anti-EXT2 autoantibody binds to EXT2, or a variant or fragment thereof.

In some embodiments, a glycoengineered polypeptide is capable of binding to: an anti-PLA2R autoantibody or a fragment or a complex thereof, an anti-THSD7A autoantibody or a fragment or a complex thereof, an anti-NELL1 autoantibody or a fragment or a complex thereof, an anti-EXT1 autoantibody or a fragment or a complex thereof, an anti-EXT2 autoantibody or a fragment or a complex thereof, or combinations thereof.

In some embodiments, a glycoengineered polypeptide disclosed herein comprises a first moiety, a second moiety and one or more additional elements. In some embodiments, a glycoengineered polypeptide comprises: an N-glycosylation site, a linker, a spacer, a signal peptide, a tag, a half-life extender or a combination thereof.

In some embodiments, a glycoengineered polypeptide comprises one or more N-glycosylation sites in a first moiety. In some embodiments, a first moiety comprises one or more N-glycosylation sites that are naturally occurring and/or one or more N-glycosylation sites that are engineered into a first moiety. In some embodiments, an engineered N-glycosylation site (also referred to herein as a glycosite or a glycotag) is or comprises the sequence of GGGGANSTAPAPAPA (SEQ ID NO: 37).

In some embodiments, a glycoengineered polypeptide comprises a linker. In some embodiments, a linker comprises a Gly-Ser linker, or an EAAAK linker. In some embodiments, a linker comprises a (Gly-Gly-Gly-Gly-Ser)n linker, wherein n is an integer between 0 to 20.

In some embodiments, a glycoengineered polypeptide comprises a spacer. In some embodiments, a spacer comprises one or more nucleotides which separates a first nucleic acid sequence from a subsequent nucleic acid sequence. In some embodiments, a spacer comprises a nucleic acid sequence which encodes one or more peptides that separates a first encoded polypeptide sequence from a subsequent encoded polypeptide sequence.

In some embodiments, a glycoengineered polypeptide comprises a signal peptide, e.g., as disclosed herein. In some embodiments, a signal peptide is a native signal peptide. In some embodiments, a signal peptide is not a native signal peptide.

In some embodiments, a signal peptide is derived from a Leishmania species. In certain embodiments, a signal peptide is derived from Leishmania tarentolae. In certain embodiments, a signal peptide is derived from Leishmania major.

In certain embodiments, the signal peptide is an invertase signal peptide derived from Leishmania tarentolae.

In certain embodiments, the signal peptide is an alkaline phosphatase signal peptide derived from Leishmania major.

In certain embodiments, a signal peptide comprises an amino acid sequence of SEQ ID NO: 21, or a portion thereof. In certain embodiments, a signal peptide comprises an amino acid sequence of SEQ ID NO: 22, or a portion thereof. In certain embodiments, a signal peptide comprises an amino acid sequence of SEQ ID NO: 44, or a portion thereof. In certain embodiments, a signal peptide is processed and removed from the glycoengineered polypeptide.

Exemplary signal peptide: SPinv, a modified signal peptide from Leishmania tarentolae invertase, SEQ ID NO: 21: MIASSVRHAVILLLVAVAMMAAVIA.

Exemplary signal peptide: SPinv, the native signal peptide from Leishmania tarentolae invertase, SEQ ID NO: 22: MIASSVRHAVILLLVAVAMMAAAVIA.

Exemplary signal peptide: native signal peptide from Leishmania major alkaline phosphatase, SEQ ID NO: 44: MASRLVRVLAAAMLVAAAVS.

In some embodiments, a glycoengineered polypeptide comprises a tag. In some embodiments, a tag is a moiety that can be used for purifying and/or identifying a glycoengineered polypeptide disclosed herein. In some embodiments, a tag comprises a His tag, a Myc tag, or a GST tag. In some embodiments, a tag comprises a cleavable tag. In some embodiments, a tag is not a glycotag.

In some embodiments, a tag is a His tag (HHHHHHHHHH; SEQ ID NO: 40).

In some embodiments, a half-life extender comprises albumin or a fragment or a variant thereof.

In some embodiments, a half-life extender comprises a Fc domain, e.g., with or without mutations in an Fc domain.

### First Moiety

In some embodiments, a glycoengineered polypeptide comprises a first moiety comprising one or more peptides that specifically binds to an anti-podocyte autoantibody or a fragment thereof.

In some embodiments, the one or more peptides comprises an epitope from a podocyte antigen which is recognized by an anti-podocyte autoantibody or a fragment thereof (e.g., an antigen binding fragment of an anti-podocyte autoantibody). In some embodiments, an epitope comprises a fragment of a podocyte antigen, e.g., an inactive fragment of a podocyte antigen and/or a soluble fragment of a podocyte antigen. In some embodiments, an epitope comprises an extracellular fragment of a podocyte antigen.

In some embodiments, a first moiety comprises a peptide that is about 5 amino acids in length to about 500 amino acids in length. In some embodiments, a first moiety comprises a peptide that is about 5 amino acids, about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, about 100 amino acids, about 200 amino acids, about 300 amino acids, about 400 amino acids, about 500 amino acids in length.

In some embodiments, a first moiety comprises a peptide that is at least 5 amino acids, at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 200 amino acids, at least 300 amino acids, at least 400 amino acids, at least 500 amino acids in length.

In some embodiments, a first moiety comprises one or more peptides that are about 50 amino acids in length to about 5000 amino acids in length in total. In some embodiments, a first moiety comprises one or more peptides that are about 50 amino acids, about 60 amino acids, about 70 amino acids, about 80 amino acids, about 90 amino acids, about 100 amino acids, about 200 amino acids, about 300 amino acids, about 400 amino acids, about 500 amino acids, about 600 amino acids, about 700 amino acids, about 800 amino acids, about 900 amino acids, about 1000 amino acids, about 1500 amino acids, about 2000 amino acids, about 2500 amino acids, about 3000 amino acids, about 3500 amino acids, about 4000 amino acids, about 4500 amino acids, about 5000 amino acids in length in total.

In some embodiments, a first moiety comprises 1, 2, 3, 4, 5, or more peptides that specifically bind to an anti-podocyte autoantibody.

In some embodiments, one or more peptides of a first moiety that specifically bind to an anti-podocyte autoantibody are the same, e.g., the one or more peptides have the same sequence. In some embodiments, the one or more peptides having the same sequence are separated by one or more intervening sequences (e.g., spacers and/or linkers). In some embodiments, the one or more peptides having the same sequence are not separated by one or more intervening sequences.

In some embodiments, one or more peptides of a first moiety that specifically bind to an anti-podocyte autoantibody are different, e.g., the one or more peptides do not have the same sequence. In some embodiments, the one or more peptides having different sequences are separated by one or more intervening sequences (e.g., spacers and/or linkers). In some embodiments, the one or more peptides different sequences are not separated by one or more intervening sequences (e.g., spacers and/or linkers).

In some embodiments, the one or more peptides having different sequences comprise one or more peptides that specifically bind to the same autoantibody. In some embodiments, the one or more peptides bind to different epitopes of an autoantibody (e.g., to different domains of the autoantibody). In some embodiments, the one or more peptides having different sequences comprise one or more peptides that specifically bind to an anti-PLA2R autoantibody or a fragment thereof. In some embodiments, the one or more peptides having different sequences comprise one or more peptides that specifically bind to an anti-THSD7A autoantibody or a fragment thereof. In some embodiments, the one or more peptides having different sequences comprise one or more peptides that specifically bind to an anti-NELL1 autoantibody or a fragment thereof. In some embodiments, the one or more peptides having different sequences comprise one or more peptides that specifically bind to an anti-EXT1 autoantibody or a fragment thereof. In some embodiments, the one or more peptides having different sequences comprise one or more peptides that specifically bind to an anti-EXT2 autoantibody or a fragment thereof.

In some embodiments, the one or more peptides having different sequences comprise one or more peptides that specifically bind to a first autoantibody (e.g., an anti-PLA2R autoantibody or a fragment thereof), one or more peptides that bind to second autoantibody (e.g., an anti-THSD7A autoantibody or a fragment thereof) and one or more peptides that bind to third autoantibody (e.g., an anti-NELL1 autoantibody or a fragment thereof). In some embodiments, the one or more peptides having different sequences are separated by one or more intervening sequences (e.g., spacers and/or linkers). In some embodiments, the one or more peptides different sequences are not separated by one or more intervening sequences (e.g., spacers and/or linkers).

In some embodiments, a linker separating one or more peptides of a first moiety comprises a Gly-Ser linker, or an EAAAK linker. In some embodiments, a linker comprises a (Gly-Gly-Gly-Gly-Ser)n linker, wherein n is an integer between 0 to 20.

In some embodiments, a spacer separating one or more peptides of a first moiety comprises 1-10 amino acid residues, or about 10-20 amino acid residues.

In some embodiments, one or more peptides that specifically bind to an anti-podocyte autoantibody are each conjugated to a second moiety.

In some embodiments, one or more peptides that specifically bind to an anti-podocyte autoantibody are not each conjugated to the second moiety.

In some embodiments, one or more peptides that specifically bind to an anti-podocyte autoantibody are conjugated to each other, e.g., are situated on one polypeptide.

In some embodiments, one or more peptides that specifically bind to an anti-podocyte autoantibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an anti-podocyte autoantibody are not separated by a protease cleavage site or an IRES, e.g., is expressed as a fusion protein.

In some embodiments, one or more peptides of a first moiety that specifically bind to an anti-podocyte autoantibody comprise an epitope that is recognized by an anti-podocyte autoantibody. In some embodiments, an epitope is a linear epitope. In some embodiments, an epitope is a conformational epitope.

In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by an anti-podocyte autoantibody, and X denotes intervening amino acid sequences with n being an integer from 0-20. In some embodiments, an intervening amino acid sequence is a spacer or a linker, e.g., as described herein.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn-[An]-Xn, wherein A1 is a first portion of an epitope, A2 is a second portion of an epitope, and An is the n-th portion of an epitope which together form a spatial epitope that is recognized by an anti-podocyte autoantibody, and X denotes intervening amino acid sequences with n being an integer from 0-20. In some embodiments, an intervening amino acid sequence is a spacer or a linker, e.g., as described herein.

In some embodiments, a first moiety comprises a plurality of epitopes, e.g., the same or different epitopes. In some embodiments, a first moiety comprises a plurality of the same epitopes, e.g., epitopes recognized by the same anti-podocyte antibody. In some embodiments, a first moiety comprises a plurality of different epitopes, e.g., epitopes recognized by different anti-podocyte antibodies. In some embodiments, the plurality of epitopes is separated by a linker, IRES or cleavage peptide.

In some embodiments, one or more peptides that specifically bind to an anti-podocyte antibody comprises an antibody agent. In some embodiments, the antibody agent comprises an antigen binding fragment. In some embodiments, the antibody agent comprises a full antibody, a Fab fragment, an scFv, a nanobody, a duobody, a single domain antibody (e.g., a VHH). In some embodiments, the antibody agent comprises a VHH, e.g., a camelid VHH or a bivalent VHH.

### Phospholipase A2 Receptor (PLA2R) and First Moiety Comprising PLA2R Peptides

M-type phospholipase A2 receptor (PLA2R) is a transmembrane polypeptide that is largely confined to glomerular podocytes rather than other human glomerular cell types in the human kidneys. PLA2R is a type I transmembrane receptor and one of the four members of the mannose receptor family in mammals. PLA2R is a receptor for secretory phospholipase A2 (sPLA2). The extracellular portion of PLA2R comprises an N-terminal cysteine-rich (CysR or ricin B) domain, a single fibronectin type 2 (FnII) domain, and eight C-type lectin-like domains (CTLDs) (Zhang P. et al. (2021) Journal of Immunology Research, volume 2021, article ID 8163298). The short cytoplasmic domain of PLA2R contains motifs that allow constitutive endocytic recycling through clathrin-coated pits. PLA2R undergoes endocytic recycling; in some embodiments, this endocytic recycling of PLA2R may provide a supply of accessible PLA2R for the formation of immune complexes at the podocyte membrane, e.g., in membranous nephropathy.

The PLA2R receptor can be cleaved into soluble form and the soluble secretory PLA2R can act as a negative regulator of sPLA2, the ligand of PLA2R (Ancian P. et al., J. Biol. Chem. 270:8963-8970(1995)).

PLA2R is the most common autoantigen in membranous nephropathy and anti-PLA2R autoantibodies are found in 70-80% patients with membranous nephropathy. Without wishing to be bound by any particular theory, it is believed that in some embodiments, the dominant epitope in PLA2R lies at the N terminus of the polypeptide, e.g., in the Cysteine rich region. Further without wishing to be bound by any particular theory, it is proposed that in the presence of reducing agents, reactivity is lost, suggesting that the antigenic epitope in PLA2R is conformation-specific and requires specific disulfide bonds (Zhang et al., 2021).

In some embodiments, PLA2R is expressed in podocytes, e.g., on the membrane of podocytes. In some embodiments, a podocyte autoantigen is a PLA2R polypeptide or a variant or fragment thereof. In some embodiments, an anti-PLA2R autoantibody or a fragment or a complex thereof is characterized in that it binds to a PLA2R polypeptide or a variant or fragment thereof (e.g., a PLA2R autoantigen). In some embodiments, a PLA2R autoantigen is human PLA2R.

A human PLA2R polypeptide sequence is provided herein as SEQ ID NO: 1, with the bolded sequence denoting the signal peptide (corresponding to Uniprot Accession Number: Q13018):

MLLSPSLLLLLLLGAPRGCAEGVAAALTPERLLEWQDKGIFVIQSESLKKCIQAGKSVL
TLENCKQANKHMLWKWVSNHGLFNIGGSGCLGLNFSAPEQPLSLYECDSTLVSLRWRC
NRKMITGPLQYSVQVAHDNTVVASRKYIHKWISYGSGGGDICEYLHKDLHTIKGNTHG
MPCMFPFQYNHQWHHECTREGREDDLLWCATTSRYERDEKWGFCPDPTSAEVGCDTI
WEKDLNSHICYQFNLLSSLSWSEAHSSCQMQGGTLLSITDETEENFIREHMSSKTVEVW
MGLNQLDEHAGWQWSDGTPLNYLNWSPEVNFEPFVEDHCGTFSSFMPSAWRSRDCEST
LPYICKKYLNHIDHEIVEKDAWKYYATHCEPGWNPYNRNCYKLQKEEKTWHEALRSCQ
ADNSALIDITSLAEVEFLVTLLGDENASETWIGLSSNKIPVSFEWSNDSSVIFTNWHTLEP
HIFPNRSQLCVSAEQSEGHWKVKNCEERLFYICKKAGHVLSDAESGCQEGWERHGGFC
YKIDTVLRSFDQASSGYYCPPALVTITNRFEQAFITSLISSVVKMKDSYFWIALQDQNDTG
EYTWKPVGQKPEPVQYTHWNTHQPRYSGGCVAMRGRHPLGRWEVKHCRHFKAMSLC
KQPVENQEKAEYEERWPFHPCYLDWESEPGLASCFKVFHSEKVLMKRTWREAEAFCEE
FGAHLASFAHIEEENFVNELLHSKFNWTEERQFWIGFNKRNPLNAGSWEWSDRTPVVSS
FLDNTYFGEDARNCAVYKANKTLLPLHCGSKREWICKIPRDVKPKIPFWYQYDVPWLFY
QDAEYLFHTFASEWLNFEFVCSWLHSDLLTIHSAHEQEFIHSKIKALSKYGASWWIGLQE
ERANDEFRWRDGTPVIYQNWDTGRERTVNNQSQRCGFISSITGLWGSEECSVSMPSICKR
KKVWLIEKKKDTPKQHGTCPKGWLYFNYKCLLLNIPKDPSSWKNWTHAQHFCAEEGG
TLVAIESEVEQAFITMNLFGQTTSVWIGLQNDDYETWLNGKPVVYSNWSPFDIINIPSHN
TTEVQKHIPLCALLSSNPNFHFTGKWYFEDCGKEGYGFVCEKMQDTSGHGVNTSDMYP
MPNTLEYGNRTYKIINANMTWYAAIKTCLMHKAQLVSITDQYHQSFLTVVLNRLGYAH
WIGLFTTDNGLNFDWSDGTKSSFTFWKDEESSLLGDCVFADSNGRWHSTACESFLQGAI
CHVPPETRQSEHPELCSETSIPWIKFKSNCYSFSTVLDSMSFEAAHEFCKKEGSNLLTIKDE
AENAFLLEELFAFGSSVQMVWLNAQFDGNNETIKWFDGTPTDQSNWGIRKPDTDYFKP
HHCVALRIPEGLWQLSPCQEKKGFICKMEADIHTAEALPEKGPSHSIIPLAVVLTLIVIVAI
CTLSFCIYKHNGGFFRRLAGFRNPYYPATNFSTVYLEENILISDLEKSDQ

Human PLA2R signal peptide: MLLSPSLLLLLLLGAPRGCA (SEQ ID NO: 14)

Human PLA2R can be encoded by the following nucleic acid sequence from the PLA2R gene (SEQ ID NO: 13)

| | |
|---|---|
| 1 | cccgagtgtc ggttcactgt ggagacagcg gtggcggagt gggtctccag ggctctgggc |
| 61 | tggcaaggcc cccggagggg tggggcgcgg aggaggctac agatccgctt ccgcgcggcg |
| 121 | gggccgggtg cttgggacgc ggctctgggc tcccgggata aggggctccc gggacaaggg |
| 181 | gctcccggag agcccagtgg ttagcgatgc tgctgtcgcc gtcgctgctg ctgctgctgc |
| 241 | tgctgggggg cgccgcgggc tgcgccgagg gtgtggcggc ggcgcttacc cccgagcggc |
| 301 | tcctggagtg gcaggataaa ggaatatttg ttatccaaag tgagagtctc aagaaatgca |
| 361 | ttcaagcagg taaatcggtt ctgaccctcg gtagaactgg aagcaagcaa gcaaacaagc |
| 421 | acatgctgtg gaaatgggtt tcaaaccatg gcctctttaa cataggaggc agcggttgcc |
| 481 | tgggcctgaa tttctccgcc ccagagcagc cattaagctt atatgaatgt gactccaccc |
| 541 | tcgtttcctt acggtggcgc tgtaacagga agatgatcac aggcccgctg cagtactctg |
| 601 | tccaggtggc gcatgacaac acagtggtgg cctcacggaa gtatattcat aagtggattt |
| 661 | cttatgggtc aggtggtgga gacatttgtg aatatctaca caaagatttg catacaatca |
| 721 | aagggaacac ccacgggatg ccgtgtatgt ttcccttcca gtataaccat cagtggcatc |
| 781 | atgaatgtac ccgtgaaggt cgggaagatg acttactgtg gtgtgccacg acaagccgtt |
| 841 | atgaaagaga tgaaaagtgg ggattttgcc ctgatcccac ctctgcagaa gtaggttgtg |
| 901 | atactatttg ggagaaggac ctcaattcac acatttgcta ccagttcaac ctgctttcat |
| 961 | ctctctcttg gagtgaggca cattcttcat gccagatgca aggaggtacg ctgttaagta |
| 1021 | ttacagatga aactgaagaa aatttcataa gggagcacat gagcagtaaa acagtggagg |
| 1081 | tgtgggttgg cctcaatcag cttgatgaag acgctggctg gcagtggtct gatggaacgc |
| 1141 | cgctcaacta tctgaattgg agcccagagg taaattttga gccatttgtt gaagatcact |
| 1201 | gtggaacatt tagttcattt atgccaagtg cctggaggag tcgggattgt gagtccacct |
| 1261 | tgccatatat atgtaaaaaa tatctaaacc acattgatca tgaaatagtt gaaaaagatg |
| 1321 | cgtggaaata ttatgctacc cactgtgagc ctggctggaa tccctacaat cgtaattgct |
| 1381 | acaaacttca gaaagaagaa aagacctggc atgaggctct gcgttcttgt caggctgata |
| 1441 | acagtgcatt aatagacata acctcattag cagaggtgga gtttcttgta accctccttg |
| 1501 | gagatgaaaa tgcatcagaa acatggattg gtttgagcag caataaaatt ccagtttcct |
| 1561 | ttgaatggtc taatgactct tcagtcatct ttactaattg gcacacactt gagccccaca |
| 1621 | tttttccaaa tagaagccag ctgtgtgtct cagcagagca gtctgaggga cactggaaag |
| 1681 | tcaaaaattg tgaagaaaga cttttttaca tttgtaaaaa agcaggccat gtcctctctg |
| 1741 | atgctgaatc aggatgtcaa gagggatggg aggagacatg tggattctgt tacaaaattg |
| 1801 | acacagtcct tcgaagcttt gaccaagctt ccagcggtta ttactgtcct cctgcacttg |
| 1861 | taaccattac aaacaggttt gaacaggctt ttattaccag tttgatcagt agtgtggtaa |
| 1921 | aaatgaagga cagttatttt tggatagctc ttcaggacca aaatgatacg ggagaataca |
| 1981 | cttggaagcc agtagggcag aaacccgagc cggtgcagta cacacactgg aacacacacc |
| 2041 | aaccgcgcta cagtggtggc tgtgttgcca tgcgaggaag gcatccactt ggtcgctggg |
| 2101 | aagtgaagca ctgtcggcac tttaaggcaa tgtccttgtg caagcagcca gttgaaaatc |
| 2161 | aggaaaaagc agagtatgaa gagagatggc cctttcaccc ctgctatttg gactgggagt |
| 2221 | cagagcctgg tctggccagt tgcttcaagg tatttcatag tgaaaaagtt ctgatgaaaa |
| 2281 | gaacatggag agaagctgaa gcattttgcg aagaatttgg agctcatctt gcaagctttg |
| 2341 | cccatattga ggaagagaat tttgtgaatg agctcttaca cccaaaattt aattggacag |
| 2401 | aagaaaggca gttctggatt ggatttaata aaagaaaccc actgaatgcc ggctcatggg |
| 2461 | agtggtctga tagaactcct gttgtctctt cgtttttaga caacacttat tttggagaag |
| 2521 | atgcaagaaa ctgtgctgtt tataagccaa acaaaacatt gctgccctta cactgtggtt |
| 2581 | ccaaacgtga atggatatgc aaaatcccaa gagatgtgaa acccaagatt ccgttctggt |
| 2641 | accagtacga tgtaccctgg ctcttttatc aggatgcaga ataccttttt catacctttg |
| 2701 | cctcagaatg gttgaacttt gagtttgtct gtagctggct gcacagtgat cttctcacaa |
| 2761 | ttcattctgc acatgagcaa gaattcatcc acagcaaaat aaaagcgcta tccaagtatg |
| 2821 | gtgcaagttg gtggattgga cttcaagaag aaagagccaa tgatgaattt cgctggagag |
| 2881 | atggaacacc agtgatatac cagaactggg acacaggaag agaaagaact gtgaataatc |
| 2941 | agagccagag atgtggcttt atttcttcta taacaggact ctggggtagt gaagagtgtt |
| 3001 | cagtttctat gcctagtatc tgtaagcgaa aaaaggtttg gctcatagag aaaaagaaag |
| 3061 | atacaccaaa acaacatgga acgtgtccca aaggatggct atattttaac tataagtgcc |
| 3121 | ttctgctgaa tatccccaaa gacccaagca gttggaagaa ctggacgcat gcccaacatt |
| 3181 | tctgtgctga agaagggggg accctggtcg ccattgaaag tgaggtggag caagctttca |
| 3241 | ttactatgaa tctttttggc cagaccacca gtgtgtggat aggtttacaa aatgatgatt |
| 3301 | atgaaacatg gctaaatgga aaacctgtgg tatattctaa ctggtctcca tttgatataa |
| 3361 | taaatattcc aagtcacaat accactgaag ttcagaaaca cattcctctc tgtgccttac |
| 3421 | tctcaagtaa tcctaatttt catttcactg gaaaatggta ttttgaagac tgtggaaagg |
| 3481 | aaggctatgg gtttgtttgt gaaaaaatgc aagatacttc tggacacggt gtaaatacat |
| 3541 | ctgatatgta tccaatgccc aataccttag aatatggaaa cagaacttac aaaataatta |
| 3601 | atgcaaatat gacttggtat gcagcaataa aaacctgcct gatgcacaaa gcacaactgg |
| 3661 | tcagcatcac agaccagtat caccagtcct tcctcactgt tgtcctcaac cggctaggat |
| 3721 | atgcccactg gattggactg ttcaccacag ataatggtct taattttgac tggtctgatg |
| 3781 | gcaccaaatc ttctttcact ttttggaaag atgaggagtc ctccctcctt ggtgactgcg |
| 3841 | tttttgccga cagcaacgga cgctggcata gcacagcctg cgactcattt ctgcaaggtg |
| 3901 | ccatttgtca tgtaccacct gaaacaagac aatctgaaca cccagagttg tgctcagaaa |
| 3961 | catctattcc ctggataaaa tttaaaagta attgctacaa gttttctaca gtcctagaca |
| 4021 | gtatgagttt tgaggctgct catgaatttt gcaaaaagga aggttctaat cttttaacaa |
| 4081 | tcaaggatga ggctgaaaat gcatttctcc tagaagagct gtttgctttt ggttcttctg |
| 4141 | tccagatggt ttggttgaat gctcaatttg atggtaacaa tgaaaccata aagtggtttg |
| 4201 | atggaactcc cacagaccag tcaaactggg gcattcggaa gccagacaca gactacttca |
| 4261 | agccccatca ttgtgttgcc ttgaggatcc ctgaaggatt atggcagcta tccccgtgtc |
| 4321 | aagaaaaaaa aggctttata tgtaaaatgg aggcagatat tcacactgca gaggcgctgc |
| 4381 | cagaaaaagg accaagtcac agcatcattc ctcttgcggt tgtactgaca ctgatagtca |
| 4441 | ttgtggccat ttgcacactt tccttctgca tatacaagca taacggtggc ttcttcagga |
| 4501 | gacttgcagg gtttcggaat ccttactatc ctgcaaccaa ctttagtaca gtatatttag |
| 4561 | aagaaaatat tctcatttct gatcttgaga agagtgacca ataataatga ggtcagagaa |
| 4621 | tgccacagac accagggtaa gtaaagaaga ctaaacagga gtctcatctg tctttccctt |
| 4681 | tacagcacag atgccattag aatgtgaatt gggtcactat tttaattatt cttgaagtga |
| 4741 | ttactggttt tgaatcttaa ccaaatcaga tgggttttga tttattcatt tccctaaact |
| 4801 | gtgatccatt cttaaaaggg gtaaattatg cattggttat ttttcagaaa gacaagaact |
| 4861 | attaaaagaa actccctatt gaaaactctg aaatcaatgc gaataatagt ttttgcatta |
| 4921 | atgtatctct actaaaattt gggggaattt taaaactaat ctggtatcta ttcagacatt |
| 4981 | tacctgcact cgtaccatta agaaagacag aaagaagcca aaaaaaatta atcttgtata |
| 5041 | tgaggggaaa aggaaagggc ttctgagagg attcttagtt gtttcttttg aattcctttt |
| 5101 | aatagcagga tttggaaaat actaatttct gtgcttaagg gtcacaggtt ctgggctctc |
| 5161 | aactgatatt taaggtgaca ttcattttta ttaggtctaa catctcaagc taaaggagaa |
| 5221 | agaaaaatac ctccttttaa atggcaaaga ccttcattag cagcacactt ttataaacac |
| 5281 | ccatatagtt aaaatgtggc cttaaacttt caattactaa atgatgatta agttggatat |
| 5341 | tttaaaatgt cttatacata agcttaaagt aatatattga aactttaaca gttgtgctgt |
| 5401 | aaaaactcat ggactttctg ggattctaaa tatattatat aatatgttac actcttaata |
| 5461 | actggtagat ataaaatgta acttggattt aaaggagtag agctaaagat ctgtattata |
| 5521 | gtctcattag taccagacag atgttgttga gaagtactaa ataaattagt aatctagata |
| 5581 | tccttattat gtaaatgagt ttaggtgttc tatttaataa actattttct gga |

In some embodiments, a PLA2R polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 1. In some embodiments, a PLA2R polypeptide is or comprises SEQ ID NO: 1. In some embodiments, a PLA2R polypeptide is or comprises SEQ ID NO: 1 without the signal peptide of SEQ ID NO: 14.

In some embodiments, a PLA2R polypeptide comprises a sequence having at least 85% identity to SEQ ID NO: 1 without the signal peptide of SEQ ID NO: 14.

In some embodiments, a PLA2R polypeptide comprising a sequence having at least 85% identity to SEQ ID NO: 1 without the signal peptide of SEQ ID NO: 14, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 1. In some embodiments, a first moiety comprises one or more peptides comprising the sequence of SEQ ID NO:1. In some embodiments, a first moiety comprises one or more peptides comprising the sequence of SEQ ID NO:1 without the signal peptide of SEQ ID NO: 14.

In some embodiments, a first moiety comprises one or more peptides comprising a sequence having at least 85% identity to SEQ ID NO: 1 without the signal peptide of SEQ ID NO: 14.

In some embodiments, a first moiety comprising one or more peptides comprising a sequence having at least 85% identity to SEQ ID NO: 1 without the signal peptide of SEQ ID NO: 14, further comprises a different signal peptide, e.g., as disclosed herein.

An engineered PLA2R polypeptide sequence is provided herein as SEQ ID NO: 38, with the bolded sequence denoting the signal peptide:

MLLSPSLLLLLLLGAPRGCAEGVAAALTPERLLEWQDKGIFVIQSESLKKCIQAGKSVL
TLENCKQANKHMLWKWVSNHGLFNIGGSGCLGLNFSAPEQPLSLYECDSTLVSLRWRC
NRKMITGPLQYSVQVAHDNTVVASRKYIHKWISYGSGGGDICEYLHKDLHTIKGNTHG
MPCMFPFQYNHQWHHECTREGREDDLLWCATTSRYERDEKWGFCPDPTSAEVGCDTI
WEKDLNSHICYQFNLLSSLSWSEAHSSCQMQGGTLLSITDETEENFIREHMSSKTVEVW
MGLNQLDEHAGWQWSDGTPLNYLNWSPEVNFEPFVEDHCGTFSSFMPSAWRSRDCEST
LPYICKKYLNHIDHEIVEKDAWKYYATHCEPGWNPYNRNCYKLQKEEKTWHEALRSCQ
ADNSALIDITSLAEVEFLVTLLGDENASETWIGLSSNKIPVSFEWSNDSSVIFTNWHTLEP
HIFPNRSQLCVSAEQSEGHWKVKNCEERLFYICKKA

In some embodiments, a PLA2R polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 38. In some embodiments, a PLA2R polypeptide is or comprises SEQ ID NO: 38. In some embodiments, a PLA2R polypeptide is or comprises SEQ ID NO: 38 without the signal peptide of SEQ ID NO: 14.

In some embodiments, a PLA2R polypeptide comprises a sequence having at least 85% identity to SEQ ID NO: 38 without the signal peptide of SEQ ID NO: 14.

In some embodiments, a PLA2R polypeptide comprising a sequence having at least 85% identity to SEQ ID NO: 38 without the signal peptide of SEQ ID NO: 14, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 38. In some embodiments, a first moiety comprises one or more peptides comprising the sequence of SEQ ID NO: 38. In some embodiments, a first moiety comprises one or more peptides comprising the sequence of SEQ ID NO:38 without the signal peptide of SEQ ID NO: 14.

In some embodiments, a first moiety comprises one or more peptides comprising a sequence having at least 85% identity to SEQ ID NO: 38 without the signal peptide of SEQ ID NO: 14.

In some embodiments, a first moiety comprising one or more peptides comprising a sequence having at least 85% identity to SEQ ID NO: 38 without the signal peptide of SEQ ID NO: 14, further comprises a different signal peptide, e.g., as disclosed herein.

In some embodiments, a PLA2R polypeptide comprises having at least 85% identity to SEQ ID NO: 39 comprises a signal peptide disclosed herein, e.g., a Leishmania derived signal peptide.

An exemplary PLA2R polypeptide sequence without a signal peptide is provided herein as SEQ ID NO: 39:

EGVAAALTPERLLEWQDKGIFVIQSESLKKCIQAGKSVLTLENCKQANKHMLWKWVSN
HGLFNIGGSGCLGLNFSAPEQPLSLYECDSTLVSLRWRCNRKMITGPLQYSVQVAHDNT
VVASRKYIHKWISYGSGGGDICEYLHKDLHTIKGNTHGMPCMFPFQYNHQWHHECTRE
GREDDLLWCATTSRYERDEKWGFCPDPTSAEVGCDTIWEKDLNSHICYQFNLLSSLSWS
EAHSSCQMQGGTLLSITDETEENFIREHMSSKTVEVWMGLNQLDEHAGWQWSDGTPLN
YLNWSPEVNFEPFVEDHCGTFSSFMPSAWRSRDCESTLPYICKKYLNHIDHEIVEKDAW
KYYATHCEPGWNPYNRNCYKLQKEEKTWHEALRSCQADNSALIDITSLAEVEFLVTLLG
DENASETWIGLSSNKIPVSFEWSNDSSVIFTNWHTLEPHIFPNRSQLCVSAEQSEGHWKV
KNCEERLFYICKKA

In some embodiments, a PLA2R polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 39. In some embodiments, a PLA2R polypeptide is or comprises SEQ ID NO: 39.

In some embodiments, a PLA2R polypeptide comprising an amino acid having at least 85% identity to SEQ ID NO: 39 comprises a signal peptide disclosed herein, e.g., a Leishmania derived signal peptide.

In some embodiments, a signal peptide is chosen from SEQ ID NO:14, SEQ ID NO: 44, SEQ ID NO: 21 or SEQ ID NO: 22.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 39. In some embodiments, a first moiety comprises one or more peptides comprising the sequence of SEQ ID NO: 39.

In some embodiments, a first moiety comprises one or more peptides comprising a sequence having at least 85% identity to SEQ ID NO: 39 and a signal peptide, e.g., as disclosed herein.

In some embodiments, a podocyte autoantigen is PLA2R polypeptide or a variant or fragment thereof.

In some embodiments, an anti-podocyte autoantibody is an anti-PLA2R autoantibody or a fragment thereof. In some embodiments, an anti-PLA2R autoantibody is characterized in that it binds to a PLA2R polypeptide or a variant or fragment thereof.

In some embodiments, a first moiety of a glycoengineered polypeptide disclosed herein comprises one or more peptides that specifically bind to an anti-PLA2R autoantibody. In some embodiments, a one or more peptides that specifically bind to an anti-PLA2R autoantibody comprises a PLA2R polypeptide, or a fragment or a variant thereof.

In some embodiments, one or more peptides that specifically bind to an anti-PLA2R autoantibody comprises a soluble fragment of a PLA2R polypeptide.

In some embodiments, one or more peptides that specifically bind to an anti-PLA2R autoantibody comprises a fragment of a PLA2R polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PLA2R polypeptide of SEQ ID NO: 1.

In some embodiments, one or more polypeptides that specifically bind to an anti-PLA2R autoantibody comprises a fragment of a PLA2R polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PLA2R polypeptide of SEQ ID NO: 1 without the signal peptide.

In some embodiments, one or more peptides that specifically bind to an anti-PLA2R autoantibody comprises a fragment of a PLA2R polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PLA2R polypeptide of SEQ ID NO: 1.

In some embodiments, one or more polypeptides that specifically bind to an anti-PLA2R autoantibody comprises a fragment of a PLA2R polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PLA2R polypeptide of SEQ ID NO: 1 without the signal peptide.

In some embodiments, one or more polypeptides that specifically bind to an anti-PLA2R autoantibody comprises a fragment of a PLA2R polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PLA2R polypeptide of SEQ ID NO: 38.

In some embodiments, one or more polypeptides that specifically bind to an anti-PLA2R autoantibody comprises a fragment of a PLA2R polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PLA2R polypeptide of SEQ ID NO: 38 without the signal peptide.

In some embodiments, one or more polypeptides that specifically bind to an anti-PLA2R autoantibody comprises a fragment of a PLA2R polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PLA2R polypeptide of SEQ ID NO: 38.

In some embodiments, one or more polypeptides that specifically bind to an anti-PLA2R autoantibody comprises a fragment of a PLA2R polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PLA2R polypeptide of SEQ ID NO: 38 without the signal peptide.

In some embodiments, one or more polypeptides that specifically bind to an anti-PLA2R autoantibody comprises a fragment of a PLA2R polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length PLA2R polypeptide of SEQ ID NO: 39.

In some embodiments, one or more polypeptides that specifically bind to an anti-PLA2R autoantibody comprises a fragment of a PLA2R polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length PLA2R polypeptide of SEQ ID NO: 39.

In some embodiments, a fragment comprises an epitope that is recognized by a PLA2R autoantibody. In some embodiments, an epitope is a linear epitope. In some embodiments, an epitope is a conformational epitope. In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by an anti-podocyte autoantibody, and X denotes intervening amino acid sequences with n being an integer from 0-20.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn-[An]-Xn, wherein A1 is a first portion of an epitope, A2 is a second portion of an epitope, and An is the n-th portion of an epitope which together form a spatial epitope that is recognized by an anti-podocyte autoantibody, and X denotes intervening amino acid sequences with n being an integer from 0-20.

In some embodiments, a first moiety comprises a plurality of epitopes, e.g., the same or different epitopes. In some embodiments, a first moiety comprises a plurality of the same epitopes, e.g., epitopes recognized by an anti-PLA2R autoantibody. In some embodiments, a first moiety comprises a plurality of different epitopes, e.g., epitopes recognized by different anti-PLA2R autoantibodies. In some embodiments, the plurality of epitopes is separated by a linker, IRES or cleavage peptide.

In some embodiments, a PLA2R polypeptide comprises the following extracellular domains: an N-terminal cysteine rich (CysR) domain, a fibronectin 2 domain, and eight C-type lectin-like domains (CTLD).

In some embodiments, a glycoengineered polypeptide comprises one or more peptides comprising the following PLA2R domains: (i) an N-terminal cysteine rich (CysR) domain or a fragment or variant thereof; (ii) a fibronectin 2 domain or a fragment or variant thereof; (iii) a CTLD1 domain, or a fragment or variant thereof; (iv) a CTLD2 domain, or a fragment or variant thereof; (v) a CTLD3 domain, or a fragment or variant thereof; (vi) a CTLD4 domain, or a fragment or variant thereof; (vii) a CTLD5 domain, or a fragment or variant thereof; (viii) a CTLD6 domain, or a fragment or variant thereof; (ix) a CTLD7 domain, or a fragment or variant thereof; (x) a CTLD8 domain, or a fragment or variant thereof; (xi) any combination of (i)-(x).

In some embodiments, a glycoengineered polypeptide comprises a PLA2R polypeptide comprising: (i) an N-terminal cysteine rich (CysR) domain or a fragment or variant thereof; (ii) a fibronectin 2 domain or a fragment or variant thereof; (iii) a CTLD1 domain, or a fragment or variant thereof; and (iv) a CTLD2 domain, or a fragment or variant thereof.

In some embodiments, a glycoengineered polypeptide comprises a PLA2R polypeptide or a fragment thereof having one or more native glycosylation sites. In some embodiments, a glycoengineered polypeptide comprises a PLA2R polypeptide or a fragment thereof having five native glycosylation sites. In some embodiments, a native glycosylation site comprises N93, N315, N433, N454 or N473.

In some embodiments, a glycoengineered polypeptide comprises a PLA2R polypeptide or a fragment thereof comprising native glycosylation sites: N93, N315, N433, N454 and N473.

In some embodiments, a glycoengineered polypeptide comprises a PLA2R polypeptide or a fragment thereof having one or more engineered glycosylation sites. In some embodiments, an engineered glycosylation site is or comprises the sequence of GGGGANSTAPAPAPA (SEQ ID NO: 37).

In some embodiments, a glycoengineered polypeptide comprises a PLA2R polypeptide or a fragment thereof having five native glycosylation sites: N93, N315, N433, N454 and N473 and one engineered glycosylation site having the sequence GGGGANSTAPAPAPA (SEQ ID NO: 37).

In some embodiments, a glycoengineered polypeptide further comprises one or more additional elements. In some embodiments, one or more additional elements comprise: (a) a linker, (b) a spacer, (c) a cleavage peptide, e.g., an IRES or a protease cleavage site, (d) a signal peptide, (e) a tag, e.g., a cleavable tag, (f) a half-life extender domain, e.g., an Fc domain or albumin, or (g) any combination of (a)-(f).

In some embodiments, a glycoengineered polypeptide comprises a tag, e.g., a His tag.

In some embodiments, a glycoengineered polypeptide comprises the following sequence (SEQ ID NO: 41): MLLSPSLLLLLLLGAPRGCAEGVAAALTPERLLEWQDKGIFVIQSESLKKCIQAGKSVLT LENCKQANKHMLWKWVSNHGLFNIGGSGCLGLNFSAPEQPLSLYECDSTLVSLRWRCN RKMITGPLQYSVQVAHDNTVVASRKYIHKWISYGSGGGDICEYLHKDLHTIKGNTHGM PCMFPFQYNHQWHHECTREGREDDLLWCATTSRYERDEKWGFCPDPTSAEVGCDTIWE KDLNSHICYQFNLLSSLSWSEAHSSCQMQGGTLLSITDETEENFIREHMSSKTVEVWMGL NQLDEHAGWQWSDGTPLNYLNWSPEVNFEPFVEDHCGTFSSFMPSAWRSRDCESTLPYI CKKYLNHIDHEIVEKDAWKYYATHCEPGWNPYNRNCYKLQKEEKTWHEALRSCQADN SALIDITSLAEVEFLVTLLGDENASETWIGLSSNKIPVSFEWSNDSSVIFTNWHTLEPHIFP NRSQLCVSAEQSEGHWKVKNCEERLFYICKKAGGGGGANSTAPAPAPAHHHHHHHHHH

In some embodiments, a glycoengineered polypeptide comprises a sequence with at least 85%, at least 90%, at least 95%, at least 99% identity to SEQ ID NO: 41. In some embodiments, a glycoengineered polypeptide comprises the sequence of SEQ ID NO: 41.

In some embodiments, one or more peptides that specifically bind to an anti-PLA2R autoantibody comprises a fragment of a PLA2R polypeptide. In some embodiments, the fragment comprises an epitope that is recognized by a PLA2R autoantibody. In some embodiments, the fragment comprises one or more domains of a PLA2R polypeptide, or a fragment or variant thereof. In some embodiments, the fragment comprises one or more additional sequences 5′ and/or 3′ to the epitope sequence.

In some embodiments, a PLA2R polypeptide comprises a CysR domain having the amino acid sequence of SEQ ID NO: 2:

KGIFVIQSESLKKCIQAGKSVLTLENCKQANKHMLWKWVSNHGLFNIGGSGCLGLNFSA
PEQPLSLYECDSTLVSLRWRCNRKMITGPLQYSVQVAHDNTVVASRKYIHKWISYGSGG
GDICEY.

In some embodiments, an epitope that is recognized by a PLA2R autoantibody comprises SEQ ID NO: 2 or a portion thereof. In some embodiments, an epitope comprises one or more additional sequences 5′ and/or 3′ to the epitope sequence.

In some embodiments, one or more peptides that specifically bind to an anti-PLA2R autoantibody comprises SEQ ID NO: 2 or a portion thereof, or a sequence with at least 90% identity to SEQ ID NO 2.

In some embodiments, an epitope that is recognized by a PLA2R autoantibody comprises SEQ ID NO: 3: WQDKGIFVIQSESLKKCIQAGKSVLTLENCK, or a portion thereof. In some embodiments, an epitope comprises one or more additional sequences 5′ and/or 3′ to the epitope sequence.

In some embodiments, one or more peptides that specifically bind to an anti-PLA2R autoantibody comprises SEQ ID NO: 3 or a portion thereof, or a sequence with at least 90% identity to SEQ ID NO 3. In some embodiments, one or more peptides that specifically bind to an anti-PLA2R autoantibody comprises SEQ ID NO: 3 but without the terminal WQD amino acid residues.

In some embodiments, a PLA2R polypeptide comprises a fibronectin 2 domain having the amino acid sequence of SEQ ID NO: 43:

THGMPCMFPFQYNHQWHHECTREGREDDLLWCATTSRYERDEKWGFCPD

In some embodiments, an epitope that is recognized by a PLA2R autoantibody comprises SEQ ID NO: 43 or a portion thereof. In some embodiments, an epitope comprises one or more additional sequences 5′ and/or 3′ to the epitope sequence.

In some embodiments, one or more peptides that specifically bind to an anti-PLA2R autoantibody comprises SEQ ID NO: 43 or a portion thereof, or a sequence with at least 90% identity to SEQ ID NO 43.

In some embodiments, a PLA2R polypeptide comprises a CTLD1 domain having the amino acid sequence of SEQ ID NO: 4:

NSHICYQFNLLSSLSWSEAHSSCQMQGGTLLSITDETEENFIREHMSSKTVEVWMGLNQL
DEHAGWQWSDGTPLNYLNWSPEVNFEPFVEDHCGTFSSFMPSAWRSRDCESTLPYICK.

In some embodiments, an epitope that is recognized by a PLA2R autoantibody comprises SEQ ID NO: 4 or a portion thereof. In some embodiments, an epitope comprises one or more additional sequences 5′ and/or 3′ to the epitope sequence.

In some embodiments, one or more peptides that specifically bind to an anti-PLA2R autoantibody comprises SEQ ID NO: 4 or a portion thereof, or a sequence with at least 90% identity to SEQ ID NO 4.

In some embodiments, a PLA2R polypeptide comprises a CTLD2 domain having the amino acid sequence of SEQ ID NO: 42:

YNRNCYKLQKEEKTWHEALRSCQADNSALIDITSLAEVEFLVTLLGDENASETWIGLSSN
KIPVSFEWSNDSSVIFTNWHTLEPHIFPNRSQLCVSAEQSEGHWKVKNCEERLFYICK.

In some embodiments, an epitope that is recognized by a PLA2R autoantibody comprises SEQ ID NO: 42 or a portion thereof. In some embodiments, an epitope comprises one or more additional sequences 5′ and/or 3′ to the epitope sequence.

In some embodiments, one or more peptides that specifically bind to an anti-PLA2R autoantibody comprises SEQ ID NO: 42 or a portion thereof, or a sequence with at least 90% identity to SEQ ID NO 42.

In some embodiments, a PLA2R polypeptide comprises a CTLD7 domain having the amino acid sequence of SEQ ID NO: 5:

YGNRTYKIINANMTWYAAIKTCLMHKAQLVSITDQYHQSFLTVVLNRLGYAHWIGLFT
TDNGLNFDWSDGTKSSFTFWKDEESSLLGDCVFADSNGRWHSTACESFLQGAIC.

In some embodiments, an epitope that is recognized by a PLA2R autoantibody comprises SEQ ID NO: 5 or a portion thereof. In some embodiments, an epitope comprises one or more additional sequences 5′ and/or 3′ to the epitope sequence.

In some embodiments, one or more peptides that specifically bind to an anti-PLA2R autoantibody comprises SEQ ID NO: 5 or a portion thereof, or a sequence with at least 90% identity to SEQ ID NO 5.

In some embodiments, a PLA2R polypeptide comprises a CTLD8 domain having the amino acid sequence of SEQ ID NO: 6:

FKSNCYSFSTVLDSMSFEAAHEFCKKEGSNLLTIKDEAENAFLLEELFAFGSSVQMVWLN
AQFDGNNETIKWFDGTPTDQSNWGIRKPDTDYFKPHHCVALRIPEGLWQLSPCQEKKGF
ICK.

In some embodiments, an epitope that is recognized by a PLA2R autoantibody comprises SEQ ID NO: 6 or a portion thereof. In some embodiments, an epitope comprises one or more additional sequences 5′ and/or 3′ to the epitope sequence.

In some embodiments, one or more peptides that specifically bind to an anti-PLA2R autoantibody comprises SEQ ID NO: 6 or a portion thereof, or a sequence with at least 90% identity to SEQ ID NO 6.

In some embodiments, one or more peptides that specifically bind to an anti-PLA2R autoantibody comprises a CysR domain and one or more CTLD domains of a PLA2R polypeptide. In some embodiments, one or more peptides that specifically bind to an anti-PLA2R autoantibody comprises (i) a CysR domain; and (ii) a CTLD1 domain, a CTLD2 domain, a CTLD3 domain, a CTLD4 domain, a CTLD5 domain, a CTLD6 domain, a CTLD7 domain, a CTLD8 domain, or combinations thereof.

In some embodiments, one or more peptides that specifically bind to an anti-PLA2R autoantibody comprises a fragment, e.g., an epitope, that is not present or not accessible in a PLA2R polypeptide from a healthy individual or an individual not at risk of developing idiopathic membranous nephropathy.

In some embodiments, one or more peptides that specifically bind to an anti-PLA2R autoantibody comprises a variant of a PLA2R polypeptide. In some embodiments, a variant is an inactive variant as compared to a wild-type PLA2R polypeptide.

In some embodiments, one or more peptides that specifically bind to an anti-PLA2R autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 1.

In some embodiments, one or more peptides that specifically bind to an anti-PLA2R autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 1 without the signal peptide.

In some embodiments, one or more peptides that specifically bind to an anti-PLA2R autoantibody comprises a full length PLA2R protein, e.g., as provided in SEQ ID NO: 1 with or without the signal peptide.

In some embodiments, a first moiety comprises 1, 2, 3, 4, 5, or more peptides that specifically bind to an anti-PLA2R autoantibody.

In some embodiments, one or more peptides of a first moiety that specifically bind to an anti-PLA2R autoantibody are the same, e.g., the one or more peptides have the same sequence of a PLA2R polypeptide, or a fragment or a variant thereof. In some embodiments, the one or more peptides having the same sequence of a PLA2R polypeptide, or a fragment or a variant thereof are separated by one or more intervening sequences (e.g., spacers and/or linkers). In some embodiments, the one or more peptides having the same sequence of a PLA2R polypeptide, or a fragment or a variant thereof are not separated by one or more intervening sequences (e.g., spacers and/or linkers).

In some embodiments, one or more peptides of a first moiety that specifically bind to an anti-PLA2R autoantibody are different, e.g., the one or more peptides do not have the same sequence of a PLA2R polypeptide, or a fragment or a variant thereof. In some embodiments, the one or more peptides having different sequences of a PLA2R polypeptide, or a fragment or a variant thereof are separated by one or more intervening sequences (e.g., spacers and/or linkers). In some embodiments, the one or more peptides different sequences of a PLA2R polypeptide, or a fragment or a variant thereof are not separated by one or more intervening sequences (e.g., spacers and/or linkers).

In some embodiments, each of the one or more peptides having different sequences of a PLA2R polypeptide, or a fragment or a variant thereof specifically bind to an anti-PLA2R autoantibody or a fragment thereof.

In some embodiments, a linker separating one or more peptides of a first moiety comprises a Gly-Ser linker, or an EAAAK linker. In some embodiments, a linker comprises a (Gly-Gly-Gly-Gly-Ser)n linker, wherein n is an integer between 0 to 20.

In some embodiments, one or more peptides that specifically bind to an anti-PLA2R autoantibody are each conjugated to a second moiety.

In some embodiments, one or more peptides that specifically bind to an anti-PLA2R autoantibody are not each conjugated to the second moiety.

In some embodiments, one or more peptides that specifically bind to an anti-PLA2R autoantibody are conjugated to each other, e.g., are situated on one polypeptide.

In some embodiments, one or more peptides that specifically bind to an anti-PLA2R autoantibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an anti-PLA2R autoantibody are not separated by a protease cleavage site or an IRES, e.g., is expressed as a fusion protein.

### Thrombospondin Type-1 Domain-Containing 7A (THSD7A) and First Moiety Comprising THSD7A Peptides

THSD7A is a type 1 transmembrane polypeptide with a large extracellular N-terminal region comprising 21 thrombospondin type 1 (TSP-1) domains, a coiled coil domain, a single-pass transmembrane domain, and a short intracellular C-terminal tail (Seifert et al., (2017) J Am Soc Nephrol 29:1536-1548). THSD7A is typically expressed in podocyte membrane (Zhang et al., 2021). The thrombospondin repeat domains are referred to either as TSP-1 through TSP-21 (or D1-D21). Alternatively, the repeat domains are referred to as repeats of TSR1 and TSR2 domains (Fresquet et al., 2019 Journal of Autoimmunity) or as THSB1-like domains or C6-like domains (Seifert et al., 2017).

THSD7A is an autoantigen in membranous nephropathy and anti-THSD7A autoantibodies are found in 1-5% of patients with membranous nephropathy (Zhang et al., 2021). Without wishing to be bound by any particular theory, it is believed that in some embodiments, the dominant epitope in THSD7A lies at the N terminus of the polypeptide, e.g., encompassing residues 48-192, e.g., TSP-1 domain and/or TSP-2 domain (Seifert et al., 2017). Further without wishing to be bound by any particular theory, it is proposed that the nonreducing form of THSD7A can be recognized by anti-THSD7A autoantibodies thus suggesting that disulfide bonds determine the antigenic epitope conformation (Zhang et al., 2021).

In some embodiments, THSD7A is expressed in podocytes, e.g., on the membrane of podocytes. In some embodiments, a podocyte autoantigen is a THSD7A polypeptide or a variant or fragment thereof. In some embodiments, an anti-THSD7A autoantibody or a fragment or a complex thereof is characterized in that it binds to a THSD7A polypeptide or a variant or fragment thereof (e.g., a THSD7A autoantigen). In some embodiments, a THSD7A autoantigen is human THSD7A.

A human THSD7A polypeptide sequence is provided herein as SEQ ID NO: 10, with the bolded sequence denoting the signal peptide:

MGLQARRWASGSRGAAGPRRGVLQLLPLPLPLPLLLLLLLRPGAGRAAAQGEAEA
PTLYLWKTGPWGRCMGDECGPGGIQTRAVWCAHVEGWTTLHTNCKQAERPNNQQNC
FKVCDWHKELYDWRLGPWNQCQPVISKSLEKPLECIKGEEGIQVREIACIQKDKDIPAED
IICEYFEPKPLLEQACLIPCQQDCIVSEFSAWSECSKTCGSGLQHRTRHVVAPPQFGGSGC
PNLTEFQVCQSSPCEAEELRYSLHVGPWSTCSMPHSRQVRQARRRGKNKEREKDRSKG
VKDPEARELIKKKRNRNRQNRQENKYWDIQIGYQTREVMCINKTGKAADLSFCQQEKL
PMTFQSCVITKECQVSEWSEWSPCSKTCHDMVSPAGTRVRTRTIRQFPIGSEKECPEFEEK
EPCLSQGDGVVPCATYGWRTTEWTECRVDPLLSQQDKRRGNQTALCGGGIQTREVYCV
QANENLLSQLSTHKNKEASKPMDLKLCTGPIPNTTQLCHIPCPTECEVSPWSAWGPCTYE
NCNDQQGKKGFKLRKRRITNEPTGGSGVTGNCPHLLEAIPCEEPACYDWKAVRLGNCEP
DNGKECGPGTQVQEVVCINSDGEEVDRQLCRDAIFPIPVACDAPCPKDCVLSTWSTWSS
CSHTCSGKTTEGKQIRARSILAYAGEEGGIRCPNSSALQEVRSCNEHPCTVYHWQTGPW
GQCIEDTSVSSFNTTTTWNGEASCSVGMQTRKVICVRVNVGQVGPKKCPESLRPETVRP
CLLPCKKDCIVTPYSDWTSCPSSCKEGDSSIRKQSRHRVIIQLPANGGRDCTDPLYEEKAC
EAPQACQSYRWKTHKWRRCQLVPWSVQQDSPGAQEGCGPGRQARAITCRKQDGGQA
GIHECLQYAGPVPALTQACQIPCQDDCQLTSWSKFSSCNGDCGAVRTRKRTLVGKSKKK
EKCKNSHLYPLIETQYCPCDKYNAQPVGNWSDCILPEGKVEVLLGMKVQGDIKECGQG
YRYQAMACYDQNGRLVETSRCNSHGYIEEACIIPCPSDCKLSEWSNWSRCSKSCGSGVK
VRSKWLREKPYNGGRPCPKLDHVNQAQVYEVVPCHSDCNQYLWVTEPWSICKVTFVN
MRENCGEGVQTRKVRCMQNTADGPSEHVEDYLCDPEEMPLGSRVCKLPCPEDCVISEW
GPWTQCVLPCNQSSFRQRSADPIRQPADEGRSCPNAVEKEPCNLNKNCYHYDYNVTDW
STCQLSEKAVCGNGIKTRMLDCVRSDGKSVDLKYCEALGLEKNWQMNTSCMVECPVN
CQLSDWSPWSECSQTCGLTGKMIRRRTVTQPFQGDGRPCPSLMDQSKPCPVKPCYRWQ
YGQWSPCQVQEAQCGEGTRTRNISCVVSDGSADDFSKVVDEEFCADIELIIDGNKNMVL
EESCSQPCPGDCYLKDWSSWSLCQLTCVNGEDLGFGGIQVRSRPVIIQELENQHLCPEQM
LETKSCYDGQCYEYKWMASAWKGSSRTVWCQRSDGINVTGGCLVMSQPDADRSCNPP
CSQPHSYCSETKTCHCEEGYTEVMSSNSTLEQCTLIPVVVLPTMEDKRGDVKTSRAVHP
TQPSSNPAGRGRTWFLQPFGPDGRLKTWVYGVAAGAFVLLIFIVSMIYLACKKPKKPQR
RQNNRLKPLTLAYDGDADM

### Human THSD7A Signal Peptide:

(SEQ ID NO: 15)
MGLQARRWASGSRGAAGPRRGVLQLLPLPLPLPLLLLLLLRPGAGRA

Human THSD7A can be encoded by the following nucleic acid sequence from the THSD7A gene (SEQ ID NO: 16)

| | |
|---|---|
| 1 | attctgcgag gaatggcagc ggcagcagca tccccgccag aggcggcggc ggcggcggca |
| 61 | gcggccacgg ccaccaccgc ggacgctatt gttccctggt gttagacgcg ctctccctcc |
| 121 | ttctcatctg aagcgaacaa tagcaggaaa gagctttgct ttctgtactc tttgaaaaga |
| 181 | cgttccaaga gcggagaaaa attccctgcc gagcgtgcta cggctctgga ccctggagtg |
| 241 | gctgcaggcg gcatggggct gcaagccagg cgctgggcgt ccgggagccg gggcgctgcg |
| 301 | gggccgcgcc ggggcgtcct gcagctgctg ccgctgccgc tgccgctgcc gctgctcctg |
| 361 | ctgctgctgc tacgcccggg cgccggcagg gctgcggcgc agggcgaggc ggaggcgccc |
| 421 | accctctatc tgtggaagac tggtccatgg ggccgatgta tgggagatga atgtggtccc |
| 481 | ggaggcatcc aaacgagggc tgtgtggtgt gctcatgtgg agggatggac tacactgcat |
| 541 | actaactgta agcaggccga gagacccaat aaccagcaga attgtttcaa agtttgcgat |
| 601 | tggcacaaag agttgtacga ctggagactg ggaccttgga atcagtgtca gcccgtgatt |
| 661 | tcaaaaagcc tagagaaacc tcttgagtgc attaaggggg aagaaggtat tcaggtgagg |
| 721 | gagatagcgt gcatccagaa agacaaagac attcctgcgg aggatatcat ctgtgagtac |
| 781 | tttgagccca agcctctcct ggagcaggct tgcctcattc cttgccagca agattgcatc |
| 841 | gtgtctgaat tttctgcctg gtccgaatgc tccaagacct gcggcagcgg gctccagcac |
| 901 | cggacgcgtc atgtggtggc gcccccgcag ttcggaggct ctggctgtcc aaacctgacg |
| 961 | gagttccagg tgtgccaatc cagtccatgc gaggccgagg agctcaggta cagcctgcat |
| 1021 | gtggggccct ggagcacctg ctcaatgccc cactcccgac aagtaagaca agcaaggaga |
| 1081 | cgcgggaaga ataaagaacg ggaaaaggac cgcagcaaag gagtaaagga tccagaagcc |
| 1141 | cgcgagctta ttaagaaaaa gagaaacaga aacaggcaga acagacaaga gaacaaatat |
| 1201 | tgggacatcc agattggata tcagaccaga gaggttatgt gcattaacaa gacggggaaa |
| 1261 | gctgctgatt taagcttttg ccagcaagag aagcttccaa tgaccttcca gtcctgtgtg |
| 1321 | atcaccaaag agtgccaggt ttccgagtgg tcagagtgga gcccctgctc aaaaacatgc |
| 1381 | catgacatgg tgtcccctgc aggcactcgt gtaaggacac gaaccatcag gcagtttccc |
| 1441 | attggcagtg aaaaggagtg tccagaattt gaagaaaaag aaccctgttt gtctcaagga |
| 1501 | gatggagttg tcccctgtgc cacgtatggc tggagaacta cagagtggac tgagtgccgt |
| 1561 | gtggaccctt tgctcagtca gcaggacaag aggcgcggca accagacggc cctctgtgga |
| 1621 | gggggcatcc agacccgaga ggtgtactgc gtgcaggcca acgaaaacct cctctcacaa |
| 1681 | ttaagtaccc acaagaacaa agaagcctca aagccaatgg acttaaaatt atgcactgga |
| 1741 | cctatcccta atactacaca gctgtgccac attccttgtc caactgaatg tgaagtttca |
| 1801 | ccttggtcag cttggggacc ttgtacttat gaaaactgta atgatcagca agggaaaaaa |
| 1861 | ggcttcaaac tgaggaagcg gcgcattacc aatgagccca ctggaggctc tggggtaacc |
| 1921 | ggaaactgcc ctcacttact ggaagccatt ccctgtgaag agcctgcctg ttatgactgg |
| 1981 | aaagcagtga gactgggaaa ctgcgagcca gataacggaa aggagtgtgg tccaggcacg |
| 2041 | caagttcaag aggttgtgtg catcaacagt gatggagaag aagttgacag acagctgtgc |
| 2101 | agagatgcca tcttccccat ccctgtggcc tgtgatgccc catgcccgaa agactgtgtg |
| 2161 | ctcagcacat ggtctacgtg gtcctcctgc tcacacacct gctcagggaa aacgacagaa |
| 2221 | gggaaacaga tacgagcacg atccattctg gcctatgcgg gtgaagaagg tggaattcgc |
| 2281 | tgtccaaata gcagtgcttt gcaagaagta cgaagctgta atgagcatcc ttgcacagtg |
| 2341 | taccactggc aaactggtcc ctggggccag tgcattgagg acacctcagt atcgtccttc |
| 2401 | aacacaacta cgacttggaa tggggaggcc tcctgctctg tcggcatgca gacaagaaaa |
| 2461 | gtcatctgtg tgcgagtcaa tgtgggccaa gtgggaccca aaaaatgtcc tgaaagcctt |
| 2521 | cgacctgaaa ctgtaaggcc ttgtctgctt ccttgtaaga aggactgtat tgtgacccca |
| 2581 | tatagtgact ggacatcatg cccctcttcg tgtaaagaag gggactccag tatcaggaag |
| 2641 | cagtctaggc atcgggtcat cattcagctg ccagccaacg ggggccgaga ctgcacagat |
| 2701 | cccctctatg aagagaaggc ctgtgaggca cctcaagcgt gccaaagcta caggtggaag |
| 2761 | actcacaaat ggcgcagatg ccaattagtc ccttggagcg tgcaacaaga cagccctgga |
| 2821 | gcacaggaag gctgtgggcc tgggcgacag gcaagagcca ttacttgtcg caagcaagat |
| 2881 | ggaggacagg ctggaatcca tgagtgccta cagtatgcag gccctgtgcc agcccttacc |
| 2941 | caggcctgcc agatcccctg ccaggatgac tgtcaattga ccagctggtc caagttttct |
| 3001 | tcatgcaatg gagactgtgg tgcagttagg accagaaagc gcactcttgt tggaaaaagt |
| 3061 | aaaaagaagg aaaaatgtaa aaattcccat ttgtatcccc tgattgagac tcagtattgt |
| 3121 | ccttgtgaca aatataatgc acaacctgtg gggaactggt cagactgtat tttaccagag |
| 3181 | ggaaaagtgg aagtgttgct gggaatgaaa gtacaaggag acatcaagga atgcggacaa |
| 3241 | ggatatcgtt accaagcaat ggcatgctac gatcaaaatg gcaggcttgt ggaaacatct |
| 3301 | agatgtaaca gccatggtta cattgaggag gcctgcatca tcccctgccc ctcagactgc |
| 3361 | aagctcagtg agtggtccaa ctggtcgcgc tgcagcaagt cctgtgggag tggtgtgaag |
| 3421 | gttcgttcta aatggctgcg tgaaaaacca tataatggag gaaggccttg ccccaaactg |
| 3481 | gaccatgtca accaggcaca ggtgtatgag gttgtcccat gccacagtga ctgcaaccag |
| 3541 | tacctatggg tcacagagcc ctggagcatc tgcaaggtga cctttgtgaa tatgcgggag |
| 3601 | aactgtggag agggcgtgca aacccgaaaa gtgagatgca tgcagaatac agcagatggc |
| 3661 | ccttctgaac atgtagagga ttacctctgt gacccagaag agatgcccct gggctctaga |
| 3721 | gtgtgcaaat taccatgccc tgaggactgt gtgatatctg aatggggtcc atggacccaa |
| 3781 | tgtgttttgc cttgcaatca aagcagtttc cggcaaaggt cagctgatcc catcagacaa |
| 3841 | ccagctgatg aaggaagatc ttgccctaat gctgttgaga aagaaccctg taacctgaac |
| 3901 | aaaaactgct accactatga ttataatgta acagactgga gtacatgtca gctgagtgag |
| 3961 | aaggcagttt gtggaaatgg aataaaaaca aggatgttgg attgtgttcg aagtgatggc |
| 4021 | aagtcagttg acctgaaata ttgtgaagcg cttggcttgg agaagaactg gcagatgaac |
| 4081 | acgtcctgca tggtggaatg ccctgtgaac tgtcagcttt ctgattggtc tccttggtca |
| 4141 | gaatgttctc aaacatgtgg cctcacagga aaaatgatcc gaagacgaac agtgacccag |
| 4201 | ccctttcaag gtgatggaag accatgccct tccctgatgg accagtccaa accctgccca |
| 4261 | gtgaagcctt gttatcggtg gcaatatggc cagtggtctc catgccaagt gcaggaggcc |
| 4321 | cagtgtggag aagggaccag aacaaggaac atttcttgtg tagtaagtga tgggtcagct |
| 4381 | gatgatttca gcaaagtggt ggatgaggaa ttctgtgctg acattgaact cattatagat |
| 4441 | ggtaataaaa atatggttct ggaggaatcc tgcagccagc cttgcccagg tgactgttat |
| 4501 | ttgaaggact ggtcttcctg gagcctgtgt cagctgacct gtgtgaatgg tgaggatcta |
| 4561 | ggctttggtg gaatacaggt cagatccaga ccggtgatta tacaagaact agagaatcag |
| 4621 | catctgtgcc cagagcagat gttagaaaca aaatcatgtt atgatggaca gtgctatgaa |
| 4681 | tataaatgga tggccagtgc ttggaagggc tcttcccgaa cagtgtggtg tcaaaggtca |
| 4741 | gatggtataa atgtaacagg gggctgcttg gtgatgagcc agcctgatgc cgacaggtct |
| 4801 | tgtaacccac cgtgtagtca accccactcg tactgtagcg agacaaaaac atgccattgt |
| 4861 | gaagaagggt acactgaagt catgtcttct aacagcaccc ttgagcaatg cacacttatc |
| 4921 | cccgtggtgg tattacccac catggaggac aaaagaggag atgtgaaaac cagtcgggct |
| 4981 | gtacatccaa cccaaccctc cagtaaccca gcaggacggg gaaggacctg gtttctacag |
| 5041 | ccatttgggc cagatgggag actaaagacc tgggtttacg gtgtagcagc tggggcattt |
| 5101 | gtgttactca tctttattgt ctccatgatt tatctagctt gcaaaaagcc aaagaaaccc |
| 5161 | caaagaaggc aaaacaaccg actgaaacct ttaaccttag cctatgatgg agatgccgac |
| 5221 | atgtaacata taacttttcc tggcaacaac cagtttcggc tttctgactt catagatgtc |
| 5281 | cagaggccac aacaaatgta tccaaactgt gtggattaaa atatatttta atttttaaaa |
| 5341 | atggcatcat aaagacaaga gtgaaaatca tactgccact ggagatattt aagacagtac |
| 5401 | cacttatata cagaccatca accgtgagaa ttataggaga tttagctgaa tacatgctgc |
| 5461 | attctgaaag ttttatgtca tcttttctga aatctaccga ctgaaaaacc actttcatct |
| 5521 | ctaaaaaata atggtggaat tggccagtta ggatgcctga tacaagaccg tctgcagtgt |
| 5581 | taatccataa aacttcctag catgaagagt ttctaccaag atctccacaa tactatggtc |
| 5641 | aaattaacat gtgtactcag ttgaatgaca cacattatgt cagattatgt acttgctaat |
| 5701 | aagcaatttt aacaatgcat aacaaataaa ctctaagcta agcagaaaat ccactgaata |
| 5761 | aattcagcat cttggtggtc gatggtagat tttattgacc tgcatttcag agacaaagcc |
| 5821 | tcttttttaa gacttcttgt ctctctccaa agtaagaatg ctggacaagt actagtgtct |
| 5881 | tagaagaacg agtcctcaag ttcagtattt tatagtggta attgtctgga aaactaattt |
| 5941 | acttgtgtta atacaatacg tttctacttt ccctgatttt caaactggtt gcctgcatct |
| 6001 | tttttgctat atggaaggca catttttgca ctatattagt gcagcacgat aggcgcttaa |
| 6061 | ccagtattgc catagaaact gcctcttttc atgtgggatg aagacatctg tgccaagagt |
| 6121 | ggcatgaaga catttgcaag ttcttgtatc ctgaagagag taaagttcag tttggatggc |
| 6181 | agcaagatga aatcagctat tacacctgct gtacacacac ttcctcatca ctgcagccat |
| 6241 | tgtgaaattg acaacatggc ggtaatttaa gtgttgaagt ccctaacccc ttaaccctct |
| 6301 | aaaaggtgga ttcctctagt tggtttgtaa ttgttctttg aaggctgttt atgactagat |
| 6361 | ttttatattt gttatctttg ttaagaaaaa aaaagaaaaa ggaactggat gtctttttaa |
| 6421 | ttttgagcag atggagaaaa taaataatgt atcaatgacc tttgtaacta aaggaaatca |
| 6481 | aaattatatg ttgatttttc tttctctctg atttcccagt ttcagattga atgtctgtct |
| 6541 | tgcaggcagt tatttcaaaa tccatagtct tttgcctttc tcactggcaa aatttgaagc |
| 6601 | aatctctctc ttcaatgtat gatttcaaaa ctaaaatttt aaaagagaag aataaatatc |
| 6661 | ttatccaaga cttcattgca tatctagagc aagagtggca aactttttct gtaaaggact |
| 6721 | atatagaaaa tattttccac aaggtctatg ttgcaacttc tcaactttgc cattctgcta |
| 6781 | atgcaatctt agtaaataaa agaatgaaca taactgtttt cctatataac ttcccttaca |
| 6841 | aaaacagcca gtaggccacc tttgcaggtc atattttgct gacccctggt ctggatgttt |
| 6901 | tttaatgcta gctctccatg gtgtcatctt tcgttttcat tcatttttca aaggtgagaa |
| 6961 | tttacagtca ttacatttcc cactgtatca gaagtagctg tattttctac agatgtagcc |
| 7021 | ctgggaatgg cacagtggaa caatatttaa ctaactcagc agacttcttg caaatgaaaa |
| 7081 | gaaaactcaa aaggtgccat taaaaagcaa taccattttc aggcaacatt attgcaacaa |
| 7141 | cctttgtgag agaggacact agcttttcag gatatttgaa aaataatata actaacatgc |
| 7201 | tattttatct taaacagctg tgttcctgta aaagtgtttt ggattagaag gtatttttac |
| 7261 | cttcatactg gagtatgaac tccccctgct gttcattaga gtatttgcaa agattacaaa |
| 7321 | agacatcagg ttaggatgtc attgtcccca aaatgaaaat atttgtatgt atttcaataa |
| 7381 | gaactaaatg cagtaagcgc taattcctca ttcaatattc actatattag gaaatgtaaa |
| 7441 | tcatttcaaa gagaacaggt agtcaggaag atgaaaattt tactttaaag caaggattta |
| 7501 | aacaaagaaa accataaaaa caaagtccat tttatctgtt ttattaaaga ggaagaaaaa |
| 7561 | ataatattgt tactttaaaa ctgtgattgt caatatatat aatcaggggt cttccagata |
| 7621 | atcctaaagt tgatctcttt gaataaaata aactccattt gtccaaaata gttaagaaag |
| 7681 | actctattgt ggttacttga aaaattcagc taaccctctt atgaaatgtg tgcaacacct |
| 7741 | attaattttg aaatgtggga tagaatacct actaaagcta tatttaaggt attgtaaaat |
| 7801 | gtcattaatt cagaaaagat tagtatcatc ctacctataa gaattaaatt agcaaatcaa |
| 7861 | aattatagaa tgttatgaaa aatacatttt attcataaat taattagatt ttacagcata |
| 7921 | tatgcatgca tatatatata tacacacaca cacacacaca cccatatata tgagagagag |
| 7981 | gaaattaggt tcaacatgac ctaatattag agaacatatt ggaaggtcac tgtttgttag |
| 8041 | gataaataaa tactggataa cctcgttaga aaggtggaaa gaggagaacg tagcaatctg |
| 8101 | gctattttgg acaaagggaa ggttattata ttcatgttta taacatgaaa atggccttat |
| 8161 | ttttgtttct gacatgagac aataatgagt taaatatttt attggaattt catcaaaacc |
| 8221 | ttatttttac tgttccttct aaaaatttgg gaggggaata tagtagtaac ttctctattg |
| 8281 | acacttttac ctcaccgagg gggtaaaaga tgtagaactg ctgttcccta gaatgaaggt |
| 8341 | ctgttgtttg gtttttaaaa aaagaagagc agaaaaagtt tgacacagta ttatatgatt |
| 8401 | ttgctggact atttcactag aaaccacgta gaataggact aactgatctc ttttgaaagg |
| 8461 | ggctgatttg cttattcatc aataactaat ccaatatgtg gacagtaatc ttaatttcaa |
| 8521 | agcctttttt gaggatacta cggtgtagct taagtgaatt taaaaagagt gcataaaagc |
| 8581 | ttgtttttaa ttacttataa caaagcacaa attacttcta acaaggcaaa tatttattga |
| 8641 | catataagac attctgctag gcattgtaaa aaaaaaaaaa aaagtaatgg cttttgtata |
| 8701 | cataggtact taagacagac atccaaaaga aggaaaaaat gcttaatgag tgaagatagc |
| 8761 | actgaaacat gttgactttt actctaggtt ttttttaga tggggttagt attttcccct |
| 8821 | acgtaatgta catgtcttta ggccacagta ttaggagttt gatttttccc tgccccaccc |
| 8881 | tcaggtaggt ggcagtggtc attgtagctt aatcagaccc ctgttaagtt cctgggcatt |
| 8941 | tcaaggtaaa ttcacgtctt ccttctcaca gaaattgaat agacctctca cacacttatt |
| 9001 | tacctccccc aacaacctgt atccttggcc ctttctgatt ttctctgctt ggtctctttc |
| 9061 | tactcttgac agctattctt acctacttcc cactaaacat gcccaatttt ttaattaaaa |
| 9121 | aaaaaagaat tcctatattt ccttccctat tagaatcaaa gtgtcactca ctcagagtgt |
| 9181 | gaacatcctg atttacacca aatccactgg tcaaagaaat gaataaaggg aaaaggaaaa |
| 9241 | atgtggaagg catgcaggaa aaaaaaaaaa aaaaaagccc ttttctgtaa aaatgtccat |
| 9301 | gcctacactt gctttagacc atcccatatc ctcctgcctc catctcgagt ctttgttcac |
| 9361 | attccacacc acaagacagt gagggaggac tgtacttttc agtaccatac agaagggaga |
| 9421 | gcctgttcta aagctgacgt gggtctcaat agtacatgag aagcacaatt cccaaatgat |
| 9481 | ctgggatctc ctgtttataa atatattacc aaattcaagt gtgggttctt cttctatttc |
| 9541 | cactgtaaga ttgagggtgt tgaaaaaaca gtagtccttg agcaaatctg tgaggaatct |
| 9601 | tgtcagaatg tgatctttcc tgggactagt ttagttgtga gaactacatc tgcttgttct |
| 9661 | gggaataagg gtctccccag gtcctgccct caggtggccc tggctgcagg acaggatttc |
| 9721 | aggtatcaga aactggtgtg tgagccaagc aaatagttat ccatttataa aaaggatggt |
| 9781 | ttgcagtgtg ttataataat tttatatcta gcagggacaa tgaaaaatgg ttatgtctac |
| 9841 | attattaaat gaatgaaact aatgctaaac agttaatttt gcatttttat ttttctgtgt |
| 9901 | ttctaccaag tcagatctta aaaatgggca gtatatgaag agctttagtt tgtgggagtc |
| 9961 | atgttttcag tgatcaaaaa tctctatatt atttagaata taattgtcag tcttgttgtg |
| 10021 | ttttgtagat ttattgtttt taagccatag tttctagatt acctactttt aaccctcaag |
| 10081 | taaacttttc cctatttttc agaaatctgt ttgtgtttat ttttagaaag taaacattta |
| 10141 | gattcctttt ctggtacttt ttttctccat ttctatcaca cagatttgaa gagatcagta |
| 10201 | tctatcattg taaaatataa ctttattaaa atttgcaatc tgtctttcta taaattatta |
| 10261 | ttccctaaat gtgtatttta tgttaattta actacctgaa tatttatttt gtttaaataa |
| 10321 | ggaggcagct gtctaagaat ttttgttaat attactttgt aaattgcgta atgtaattta |
| 10381 | ttttacatta tgtacatttc ctttttaacg cacaaaatgt ctatgtacaa ttaactacag |
| 10441 | atttgcaaat aatgtcacta agctttattc agtcaataca gatggcatgt gaagatgtaa |
| 10501 | tatgcttttt tacattttca tatgggttat ttgtaaataa ctatattgct gacaaacata |
| 10561 | aatacggaaa tcattttcat tctaatcatg tgtatctgtg ttttgatttg caccaattgc |
| 10621 | ttcttaaata tggattaaaa tttttttcca gtgaa |

In some embodiments, a THSD7A polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 10. In some embodiments, a THSD7A polypeptide is or comprises SEQ ID NO: 10. In some embodiments, a THSD7A polypeptide is or comprises SEQ ID NO: 10 without the signal peptide.

In some embodiments, a THSD7A polypeptide comprises a sequence having at least 85% identity to SEQ ID NO: 10 without the signal peptide of SEQ ID NO: 15.

In some embodiments, a THSD7A polypeptide comprising a sequence having at least 85% identity to SEQ ID NO: 10 without the signal peptide of SEQ ID NO: 15, further comprises a different signal peptide, e.g., as disclosed herein.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 10. In some embodiments, a first moiety comprises one or more peptides comprising the sequence of SEQ ID NO: 10. In some embodiments, a first moiety comprises one or more peptides comprising the sequence of SEQ ID NO: 10 without the signal peptide of SEQ ID NO: 15.

In some embodiments, a first moiety comprises one or more peptides comprising a sequence having at least 85% identity to SEQ ID NO: 10 without the signal peptide of SEQ ID NO: 15.

In some embodiments, a first moiety comprising one or more peptides comprising a sequence having at least 85% identity to SEQ ID NO: 10 without the signal peptide of SEQ ID NO: 15, further comprises a different signal peptide, e.g., as disclosed herein. In some embodiments, a signal peptide is chosen from SEQ ID NO: 15, SEQ ID NO: 44, SEQ ID NO: 21 or SEQ ID NO: 22.

In some embodiments, a podocyte autoantigen is THSD7A polypeptide or a variant or fragment thereof.

In some embodiments, an anti-podocyte autoantibody is an anti-THSD7A autoantibody or a fragment thereof. In some embodiments, an anti-THSD7A autoantibody is characterized in that it binds to a THSD7A polypeptide or a variant or fragment thereof.

In some embodiments, a first moiety of a glycoengineered polypeptide disclosed herein comprises one or more peptides that specifically bind to an anti-THSD7A autoantibody. In some embodiments, a one or more peptides that specifically bind to an anti-THSD7A autoantibody comprises a THSD7A polypeptide, or a fragment or a variant thereof.

In some embodiments, one or more peptides that specifically bind to an anti-THSD7A autoantibody comprises a soluble fragment of a THSD7A polypeptide.

In some embodiments, one or more peptides that specifically bind to an anti-THSD7A autoantibody comprises a fragment of a THSD7A polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length THSD7A polypeptide of SEQ ID NO: 10.

In some embodiments, one or more peptides that specifically bind to an anti-THSD7A autoantibody comprises a fragment of a THSD7A polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length THSD7A polypeptide of SEQ ID NO: 10 without the signal peptide.

In some embodiments, one or more peptides that specifically bind to an anti-THSD7A autoantibody comprises a fragment of a THSD7A polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length THSD7A polypeptide of SEQ ID NO: 10.

In some embodiments, one or more peptides that specifically bind to an anti-THSD7A autoantibody comprises a fragment of a THSD7A polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length THSD7A polypeptide of SEQ ID NO: 10 without the signal peptide.

In some embodiments, a fragment comprises an epitope that is recognized by a THSD7A autoantibody. In some embodiments, an epitope is a linear epitope. In some embodiments, an epitope is a conformational epitope. In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by an anti-podocyte autoantibody, and X denotes intervening amino acid sequences with n being an integer from 0-20.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn-[An]-Xn, wherein A1 is a first portion of an epitope, A2 is a second portion of an epitope, and An is the n-th portion of an epitope which together form a spatial epitope that is recognized by an anti-podocyte autoantibody, and X denotes intervening amino acid sequences with n being an integer from 0-20.

In some embodiments, a first moiety comprises a plurality of epitopes, e.g., the same or different epitopes. In some embodiments, a first moiety comprises a plurality of the same epitopes, e.g., epitopes recognized by an anti-THSD7A autoantibody. In some embodiments, a first moiety comprises a plurality of different epitopes, e.g., epitopes recognized by different anti-THSD7A autoantibodies. In some embodiments, the plurality of epitopes is separated by a linker, IRES or cleavage peptide.

In some embodiments, a THSD7A polypeptide comprises the following extracellular domains: 21 thrombospondin type 1 domains (TSD) and a coiled coil domain. In some embodiments, the TSD domains comprise a TSR1 domain and/or a TSR2 domain.

In some embodiments, one or more peptides that specifically bind to an anti-THSD7A autoantibody comprises a fragment of a THSD7A polypeptide. In some embodiments, the fragment comprises an epitope that is recognized by a THSD7A autoantibody. In some embodiments, the fragment comprises one or more domains of a THSD7A polypeptide, or a fragment or variant thereof. In some embodiments, the fragment comprises one or more additional sequences 5′ and/or 3′ to the epitope sequence.

In some embodiments, a THSD7A polypeptide comprises a TSP-1 domain having the amino acid sequence of SEQ ID NO: 11:

TLYLWKTGPWGRCMGDECGPGGIQTRAVWCAHVEGWTTLHTNCKQAERP
NNQQNCFKVCD.

In some embodiments, an epitope that is recognized by a THSD7A autoantibody comprises SEQ ID NO: 11 or a portion thereof. In some embodiments, an epitope comprises one or more additional sequences 5′ and/or 3′ to the epitope sequence.

In some embodiments, one or more peptides that specifically bind to an anti-THSD7A autoantibody comprises SEQ ID NO: 11 or a portion thereof, or a sequence with at least 90% identity to SEQ ID NO 11.

In some embodiments, a THSD7A polypeptide comprises the amino acid sequence of SEQ ID NO: 12: GPGGIQTRAVWCAHVEGWTTLHTNCKQA.

In some embodiments, an epitope that is recognized by a THSD7A autoantibody comprises SEQ ID NO: 12 or a portion thereof. In some embodiments, an epitope comprises one or more additional sequences 5′ and/or 3′ to the epitope sequence.

In some embodiments, one or more peptides that specifically bind to an anti-THSD7A autoantibody comprises SEQ ID NO: 12 or a portion thereof, or a sequence with at least 90% identity to SEQ ID NO 12.

In some embodiments, a THSD7A polypeptide comprises a TSP-2 domain having the amino acid sequence of SEQ ID NO: 17:

ELYDWRLGPWNQCQPVISKSLEKPLECIKGEEGIQVREIACIQKDKDIPAEDIICEYFEPKP
LLEQACLIPCQ.

In some embodiments, an epitope that is recognized by a THSD7A autoantibody comprises SEQ ID NO: 17 or a portion thereof. In some embodiments, an epitope comprises one or more additional sequences 5′ and/or 3′ to the epitope sequence.

In some embodiments, one or more peptides that specifically bind to an anti-THSD7A autoantibody comprises SEQ ID NO: 17 or a portion thereof, or a sequence with at least 90% identity to SEQ ID NO 17.

In some embodiments, one or more peptides that specifically bind to an anti-THSD7A autoantibody comprises one or more TSP domains. In some embodiments, one or more peptides that specifically bind to an anti-THSD7A autoantibody comprises a TSP-1 domain of SEQ ID NO: 11 and a TSP-2 domain of SEQ ID NO: 17.

In some embodiments, a THSD7A polypeptide comprises SEQ ID NO: 18:

AAQGEAEAPTLYLWKTGPWGRCMGDECGPGGIQTRAVWCAHVEGWTTLHTNCKQAE
RPNNQQNCFKVCDWHKELYDWRLGPWNQCQPVISKSLEKPLECIKGEEGIQVREIACIQ
KDKDIPAEDIICEYFEPKPLLEQACLIPCQ

In some embodiments, an epitope that is recognized by a THSD7A autoantibody comprises SEQ ID NO: 18 or a portion thereof. In some embodiments, an epitope comprises one or more additional sequences 5′ and/or 3′ to the epitope sequence.

In some embodiments, one or more peptides that specifically bind to an anti-THSD7A autoantibody comprises SEQ ID NO: 18 or a portion thereof, or a sequence with at least 90% identity to SEQ ID NO 18.

In some embodiments, one or more peptides that specifically bind to an anti-THSD7A autoantibody comprises a fragment, e.g., an epitope, that is not present or not accessible in a THSD7A polypeptide from a healthy individual or an individual not at risk of developing idiopathic membranous nephropathy.

In some embodiments, one or more peptides that specifically bind to an anti-THSD7A autoantibody comprises a variant of a THSD7A polypeptide. In some embodiments, a variant is an inactive variant as compared to a wild-type THSD7A polypeptide.

In some embodiments, one or more peptides that specifically bind to an anti-THSD7A autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 10.

In some embodiments, one or more peptides that specifically bind to an anti-THSD7A autoantibody comprises a full length THSD7A protein, e.g., as provided in SEQ ID NO: 10 with or without the signal peptide.

In some embodiments, a first moiety comprises 1, 2, 3, 4, 5, or more peptides that specifically bind to an anti-THSD7A autoantibody.

In some embodiments, one or more peptides of a first moiety that specifically bind to an anti-THSD7A autoantibody are the same, e.g., the one or more peptides have the same sequence of a THSD7A polypeptide, or a fragment or a variant thereof. In some embodiments, the one or more peptides having the same sequence of a THSD7A polypeptide, or a fragment or a variant thereof are separated by one or more intervening sequences (e.g., spacers and/or linkers). In some embodiments, the one or more peptides having the same sequence of a THSD7A polypeptide, or a fragment or a variant thereof are not separated by one or more intervening sequences (e.g., spacers and/or linkers).

In some embodiments, one or more peptides of a first moiety that specifically bind to an anti-THSD7A autoantibody are different, e.g., the one or more peptides do not have the same sequence of a THSD7A polypeptide, or a fragment or a variant thereof. In some embodiments, the one or more peptides having different sequences of a THSD7A polypeptide, or a fragment or a variant thereof are separated by one or more intervening sequences (e.g., spacers and/or linkers). In some embodiments, the one or more peptides different sequences of a THSD7A polypeptide, or a fragment or a variant thereof are not separated by one or more intervening sequences (e.g., spacers and/or linkers).

In some embodiments, each of the one or more peptides having different sequences of a THSD7A polypeptide, or a fragment or a variant thereof specifically bind to an anti-THSD7A autoantibody or a fragment thereof.

In some embodiments, a linker separating one or more peptides of a first moiety comprises a Gly-Ser linker, or an EAAAK linker. In some embodiments, a linker comprises a (Gly-Gly-Gly-Gly-Ser)n linker, wherein n is an integer between 0 to 20.

In some embodiments, one or more peptides that specifically bind to an anti-THSD7A autoantibody are each conjugated to a second moiety.

In some embodiments, one or more peptides that specifically bind to an anti-THSD7A autoantibody are not each conjugated to the second moiety.

In some embodiments, one or more peptides that specifically bind to an anti-THSD7A autoantibody are conjugated to each other, e.g., are situated on one polypeptide.

In some embodiments, one or more peptides that specifically bind to an anti-THSD7A autoantibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an anti-THSD7A autoantibody are not separated by a protease cleavage site or an IRES, e.g., is expressed as a fusion protein.

### Neural Epidermal Growth Factor-Like 1 Protein (NELL-1) and First Moiety Comprising NELL-1 Peptides

Neural epidermal growth factor-like 1 protein (NELL-1) is a secreted polypeptide with epidermal growth factor (EFG)-like repeats. NELL-1 is highly expressed in in osteoblasts and promotes bone regeneration (Ronco et al. Curr Opin Neprol Hypertens, 2021). NELL-1 protein is an 810 amino acid protein comprising the following domains from N terminus to C terminus: a thrombospondin-1 like (TSPN) domain, a coiled coil domain, two von Willebrand factor, type C (VWFC) domains, six EGF domains, and two additional VWFC domains (Paksava M et al., (2017) Genes & Diseases 4(3) pp 127-137). At least one report has identified Apoptosis Related Protein 3 (ARP3) as a NELL1 binding partner (Zou X. et al., FEBS Letters (2011) 2410-2418).

A subset of membranous nephropathy is associated with accumulation and co-localization of NELL-1 and IgG along the glomerular basement membrane. NELL1 is an autoantigen in membranous nephropathy and anti-NELL1 autoantibodies are found in 1-5% of patients with membranous nephropathy.

In some embodiments, NELL1 is expressed in podocytes, e.g., on the membrane of podocytes. In some embodiments, a podocyte autoantigen is a NELL1 polypeptide or a variant or fragment thereof. In some embodiments, an anti-NELL1 autoantibody or a fragment or a complex thereof is characterized in that it binds to a NELL1 polypeptide or a variant or fragment thereof (e.g., a NELL1 autoantigen). In some embodiments, a NELL1 autoantigen is human NELL1.

A human NELL1 polypeptide sequence is provided herein as SEQ ID NO: 9, with the bolded sequence denoting the signal peptide:

MPMDLILVVWFCVCTARTVVGFGMDPDLQMDIVTELDLVNTTLGVAQ
VSGMHNASKAFLFQDIEREIHAAPHVSEKLIQLFRNKSEFTILATVQQKPSTSGVILSIREL
EHSYFELESSGLRDEIRYHYIHNGKPRTEALPYRMADGQWHKVALSVSASHLLLHVDCN
RIYERVIDPPDTNLPPGINLWLGQRNQKHGLFKGIIQDGKIIFMPNGYITQCPNLNHTCPTC
SDFLSLVQGIMDLQELLAKMTAKLNYAETRLSQLENCHCEKTCQVSGLLYRDQDSWVD
GDHCRNCTCKSGAVECRRMSCPPLNCSPDSLPVHIAGQCCKVCRPKCIYGGKVLAEGQR
ILTKSCRECRGGVLVKITEMCPPLNCSEKDHILPENQCCRVCRGHNFCAEGPKCGENSEC
KNWNTKATCECKSGYISVQGDSAYCEDIDECAAKMHYCHANTVCVNLPGLYRCDCVP
GYIRVDDFSCTEHDECGSGQHNCDENAICTNTVQGHSCTCKPGYVGNGTICRAFCEEGC
RYGGTCVAPNKCVCPSGFTGSHCEKDIDECSEGIIECHNHSRCVNLPGWYHCECRSGFH
DDGTYSLSGESCIDIDECALRTHTCWNDSACINLAGGFDCLCPSGPSCSGDCPHEGGLKH
NGQVWTLKEDRCSVCSCKDGKIFCRRTACDCQNPSADLFCCPECDTRVTSQCLDQNGH
KLYRSGDNWTHSCQQCRCLEGEVDCWPLTCPNLSCEYTAILEGECCPRCVSDPCLADNI
TYDIRKTCLDSYGVSRLSGSVWTMAGSPCTTCKCKNGRVCCSVDFECLQNN

Human NELL1 signal peptide: MPMDLILVVWFCVCTARTVVG (SEQ ID NO: 19)

Human NELL1 can be encoded by the following nucleic acid sequence from the NELL1 gene (SEQ ID NO: 20)

| | |
|---|---|
| 1 | ggggctgcct tcccgggcgc atatgcgagc gcagcacccg gcgctgccga gccacctccc |
| 61 | ccgccgcccg ctagcaagtt tggcggctcc aagccaggcg cgcctcagga tccaggctca |
| 121 | tttgcttcca cctagcttcg gtgccccctg ctaggcgggg accctcgaga gcgatgccga |
| 181 | tggatttgat tttagttgtg tggttctgtg tgtgcactgc caggacagtg gtgggctttg |
| 241 | ggatggaccc tgaccttcag atggatatcg tcaccgagct tgaccttgtg aacaccaccc |
| 301 | ttggagttgc tcaggtgtct ggaatgcaca atgccagcaa agcattttta tttcaagaca |
| 361 | tagaaagaga gatccatgca gctcctcatg tgagtgagaa attaattcag ctgttccgga |
| 421 | acaagagtga attcaccatt ttggccactg tacagcagaa gccatccact tcaggagtga |
| 481 | tactgtccat tcgagaactg gagcacagct attttgaact ggagagcagt ggcctgaggg |
| 541 | atgagattcg gtatcactac atacacaatg ggaagccaag gacagaggca cttccttacc |
| 601 | gcatggcaga tggacaatgg cacaaggttg cactgtcagt tagcgcctct catctcctgc |
| 661 | tccatgtcga ctgtaacagg atttatgagc gtgtgataga ccctccagat accaaccttc |
| 721 | ccccaggaat caatttatgg cttggccagc gcaaccaaaa gcatggctta ttcaaaggga |
| 781 | tcatccaaga tgggaagatc atctttatgc cgaatggata tataacacag tgtccaaatc |
| 841 | taaatcacac ttgcccaacc tgcagtgatt tcttaagcct ggtgcaagga ataatggatt |
| 901 | tacaagagct tttggccaag atgactgcaa aactaaatta tgcagagaca agacttagtc |
| 961 | aattggaaaa ctgtcattgt gagaagactt gtcaagtgag tggactgctc tatcgagatc |
| 1021 | aagactcttg ggtagatggt gaccattgca ggaactgcac ttgcaaaagt ggtgccgtgg |
| 1081 | aatgccgaag gatgtcctgt ccccctctca attgctcccc agactccctc ccagtgcaca |
| 1141 | ttgctggcca gtgctgtaag gtctgccgac caaaatgtat ctatggagga aaagttcttg |
| 1201 | cagaaggcca gcggatttta accaagagct gtcgggaatg ccgaggtgga gttttagtaa |
| 1261 | aaattacaga aatgtgtcct cctttgaact gctcagaaaa ggatcacatt cttcctgaga |
| 1321 | atcagtgctg ccgtgtctgt agaggtcata acttttgtgc agaaggacct aaatgtggtg |
| 1381 | aaaactcaga gtgcaaaaac tggaatacaa aagctacttg tgagtgcaag agtggttaca |
| 1441 | tctctgtcca gggagactct gcctactgtg aagatattga tgagtgtgca gctaagatgc |
| 1501 | attactgtca tgccaatact gtgtgtgtca accttcctgg gttatatcgc tgtgactgtg |
| 1561 | tcccaggata cattcgtgtg gatgacttct cttgtacaga acacgatgaa tgtggcagcg |
| 1621 | gccagcacaa ctgtgatgag aatgccatct gcaccaacac tgtccaggga cacagctgca |
| 1681 | cctgcaaacc gggctacgtg gggaacggga ccatctgcag agctttctgt gaagagggct |
| 1741 | gcagatacgg tggaacgtgt gtggctccca acaaatgtgt ctgtccatct ggattcacag |
| 1801 | gaagccactg cgagaaagac attgatgaat gtgccttaag aactcacacc tgttggaacg |
| 1861 | attctgcctg catcaacctg gcagggggct ttgactgtct ctgcccctct gggccctcct |
| 1921 | gctctggtga ctgtcctcat gaaggggggc tgaagcacaa tggccaggtg tggaccttga |
| 1981 | aagaagacag gtgttctgtc tgctcctgca aggatggcaa gatattctgc cgacggacag |
| 2041 | cttgtgattg ccagaatcca agtgctgacc tattctgttg cccagaatgt gacaccagag |
| 2101 | tcacaagtca atgtttagac caaaatggtc acaagctgta tcgaagtgga gacaattgga |
| 2161 | cccatagctg tcagcagtgt cggtgtctgg aaggagaggt agattgctgg ccactcactt |
| 2221 | gccccaactt gagctgtgag tatacagcta tcttagaagg ggaatgttgt ccccgctgtg |
| 2281 | tcagtgaccc ctgcctagct gataacatca cctatgacat cagaaaaact tgcctggaca |
| 2341 | gctatggtgt ttcacggctt agtggctcag tgtggacgat ggctggatct ccctgcacaa |
| 2401 | cctgtaaatg caagaatgga agagtctgtt gttctgtgga ttttgagtgt cttcaaaata |
| 2461 | attgaagtat ttacagtgga ctcaacgcag aagaatggac gaaatgacca tccaacgtga |
| 2521 | ttaaggatag gaatcggtag tttggttttt ttgtttgttt tgttttttta accacagata |
| 2581 | attgccaaag tttccacctg aggacggtgt ttggaggttg ccttttggac ctaccacttt |
| 2641 | gctcattctt gctaacctag tctaggtgac ctacagtgcc gtgcatttaa gtcaatggtt |
| 2701 | gttaaaagaa gtttcccgtg ttgtaaatca tgtttccctt atcagatcat ttgcaaatac |
| 2761 | atttaaatga tctcatggta aatgttgatg tattttttgg tttattttgt gtactaacat |
| 2821 | aatagagaga gactcagctc cttttattta ttttgttgat ttatggatca aattctaaaa |
| 2881 | taaagttgcc tgttgtgact tttgtcccat ctactgcata cttagtgctg agatccctgt |
| 2941 | aaaatgtttt gatgaaaata tgtatgtaga gtccagtcgc attatacata catttcatag |
| 3001 | tgctgaacct tcttaaatgc ctactcattc agcttaaaca ggctgaagcc aagtatgaca |
| 3061 | aagaggggaa gggccaaaaa cataatcaaa gaataatttt aaagagaatt cttgtctctc |
| 3121 | ttgcaaaaa |

In some embodiments, a NELL1 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 9. In some embodiments, a NELL1 polypeptide is or comprises SEQ ID NO: 9. In some embodiments, a NELL1 polypeptide is or comprises SEQ ID NO: 9 without the signal peptide.

In some embodiments, a NELL1 polypeptide comprises a sequence having at least 85% identity to SEQ ID NO: 9 without the signal peptide of SEQ ID NO: 19.

In some embodiments, a NELL1 polypeptide comprising a sequence having at least 85% identity to SEQ ID NO: 9 without the signal peptide of SEQ ID NO: 19, further comprises a different signal peptide, e.g., as disclosed herein. In some embodiments, a signal peptide is chosen from SEQ ID NO:19, SEQ ID NO: 44, SEQ ID NO: 21 or SEQ ID NO: 22.

In some embodiments, a glycoengineered polypeptide comprises a first moiety comprising one or more peptides comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 9. In some embodiments, a first moiety comprises one or more peptides comprising the sequence of SEQ ID NO: 9. In some embodiments, a first moiety comprises one or more peptides comprising the sequence of SEQ ID NO:9 without the signal peptide of SEQ ID NO: 19.

In some embodiments, a first moiety comprises one or more peptides comprising a sequence having at least 85% identity to SEQ ID NO: 9 without the signal peptide of SEQ ID NO: 19.

In some embodiments, a first moiety comprising one or more peptides comprising a sequence having at least 85% identity to SEQ ID NO: 9 without the signal peptide of SEQ ID NO: 19, further comprises a different signal peptide, e.g., as disclosed herein.

In some embodiments, a podocyte autoantigen is NELL1 polypeptide or a variant or fragment thereof.

In some embodiments, an anti-podocyte autoantibody is an anti-NELL1 autoantibody or a fragment thereof. In some embodiments, an anti-NELL1 autoantibody is characterized in that it binds to a NELL1 polypeptide or a variant or fragment thereof.

In some embodiments, a first moiety of a glycoengineered polypeptide disclosed herein comprises one or more peptides that specifically bind to an anti-NELL1 autoantibody. In some embodiments, a one or more peptides that specifically bind to an anti-NELL1 autoantibody comprises a NELL1 polypeptide, or a fragment or a variant thereof.

In some embodiments, one or more peptides that specifically bind to an anti-NELL1 autoantibody comprises a soluble fragment of a NELL1 polypeptide.

In some embodiments, one or more peptides that specifically bind to an anti-NELL1 autoantibody comprises a fragment of a NELL1 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length NELL1 polypeptide of SEQ ID NO: 9.

In some embodiments, one or more peptides that specifically bind to an anti-NELL1 autoantibody comprises a fragment of a NELL1 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length NELL1 polypeptide of SEQ ID NO: 9 without the signal peptide.

In some embodiments, one or more peptides that specifically bind to an anti-NELL1 autoantibody comprises a fragment of a NELL1 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length NELL1 polypeptide of SEQ ID NO: 9.

some embodiments, one or more peptides that specifically bind to an anti-NELL1 autoantibody comprises a fragment of a NELL1 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length NELL1 polypeptide of SEQ ID NO: 9 without the signal peptide.

In some embodiments, a fragment comprises an epitope that is recognized by a NELL1 autoantibody. In some embodiments, an epitope is a linear epitope. In some embodiments, an epitope is a conformational epitope. In some embodiments, an epitope is or comprises a single continuous epitope. In some embodiments, an epitope comprises one or more additional amino acid residues, e.g., on the 5′ end and/or the 3′ end of the epitope.

In some embodiments, an epitope comprises one or more sequences separated by one or more intervening amino acid sequences configured such that the one or more sequences form a single epitope, e.g., spatially form an epitope when expressed and folded into a polypeptide conformation. In some embodiments, an intervening amino acid sequence comprises a linker and/or a spacer. For example, an epitope comprising one or more sequences separated by one or more intervening amino acid sequences has the following structure: Xn-[A1]-Xn-[A2]-Xn, wherein A1 is a first portion of an epitope and A2 is a second portion of an epitope which together form a spatial epitope that is recognized by an anti-podocyte autoantibody, and X denotes intervening amino acid sequences with n being an integer from 0-20.

In some embodiments, an epitope that is formed by one or more sequences can be broken up into 3, 4, 5, or more fragments. For example, in such embodiments, the polypeptide may comprise the following structure: Xn-[A1]-Xn-[A2]-Xn-[An]-Xn, wherein A1 is a first portion of an epitope, A2 is a second portion of an epitope, and An is the n-th portion of an epitope which together form a spatial epitope that is recognized by an anti-podocyte autoantibody, and X denotes intervening amino acid sequences with n being an integer from 0-20.

In some embodiments, a first moiety comprises a plurality of epitopes, e.g., the same or different epitopes. In some embodiments, a first moiety comprises a plurality of the same epitopes, e.g., epitopes recognized by an anti-NELL1 autoantibody. In some embodiments, a first moiety comprises a plurality of different epitopes, e.g., epitopes recognized by different anti-NELL1 autoantibodies. In some embodiments, the plurality of epitopes is separated by a linker, IRES or cleavage peptide.

In some embodiments, a NELL1 polypeptide comprises the following domains a TSPN domain, a coiled coil domain, one or more Willebrand factor, type C (VWFC) domains, and/or one or more EGF domains.

In some embodiments, one or more peptides that specifically bind to an anti-NELL1 autoantibody comprises a fragment of a NELL1 polypeptide. In some embodiments, the fragment comprises an epitope that is recognized by a NELL1 autoantibody. In some embodiments, the fragment comprises one or more domains of a NELL1 polypeptide, or a fragment or variant thereof. In some embodiments, the fragment comprises one or more additional sequences 5′ and/or 3′ to the epitope sequence.

In some embodiments, one or more peptides that specifically bind to an anti-NELL1 autoantibody comprises a fragment, e.g., an epitope, that is not present or not accessible in a NELL1 polypeptide from a healthy individual or an individual not at risk of developing idiopathic membranous nephropathy.

In some embodiments, one or more peptides that specifically bind to an anti-NELL1 autoantibody comprises a variant of a NELL1 polypeptide. In some embodiments, a variant is an inactive variant as compared to a wild-type NELL1 polypeptide.

In some embodiments, one or more peptides that specifically bind to an anti-NELL1 autoantibody comprises a contiguous chain of amino acids comprising at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, of the amino acid of SEQ ID NO: 9.

In some embodiments, one or more peptides that specifically bind to an anti-NELL1 autoantibody comprises a full length NELL1 protein, e.g., as provided in SEQ ID NO: 9 with or without the signal peptide.

In some embodiments, a first moiety comprises 1, 2, 3, 4, 5, or more peptides that specifically bind to an anti-NELL1 autoantibody.

In some embodiments, one or more peptides of a first moiety that specifically bind to an anti-NELL1 autoantibody are the same, e.g., the one or more peptides have the same sequence of a NELL1 polypeptide, or a fragment or a variant thereof. In some embodiments, the one or more peptides having the same sequence of a NELL1 polypeptide, or a fragment or a variant thereof are separated by one or more intervening sequences (e.g., spacers and/or linkers). In some embodiments, the one or more peptides having the same sequence of a NELL1 polypeptide, or a fragment or a variant thereof are not separated by one or more intervening sequences (e.g., spacers and/or linkers).

In some embodiments, one or more peptides of a first moiety that specifically bind to an anti-NELL1 autoantibody are different, e.g., the one or more peptides do not have the same sequence of a NELL1 polypeptide, or a fragment or a variant thereof. In some embodiments, the one or more peptides having different sequences of a NELL1 polypeptide, or a fragment or a variant thereof are separated by one or more intervening sequences (e.g., spacers and/or linkers). In some embodiments, the one or more peptides different sequences of a NELL1 polypeptide, or a fragment or a variant thereof are not separated by one or more intervening sequences (e.g., spacers and/or linkers).

In some embodiments, each of the one or more peptides having different sequences of a NELL1 polypeptide, or a fragment or a variant thereof specifically bind to an anti-NELL1 autoantibody or a fragment thereof.

In some embodiments, a linker separating one or more peptides of a first moiety comprises a Gly-Ser linker, or an EAAAK linker. In some embodiments, a linker comprises a (Gly-Gly-Gly-Gly-Ser)n linker, wherein n is an integer between 0 to 20.

In some embodiments, one or more peptides that specifically bind to an anti-NELL1 autoantibody are each conjugated to a second moiety.

In some embodiments, one or more peptides that specifically bind to an anti-NELL1 autoantibody are not each conjugated to the second moiety.

In some embodiments, one or more peptides that specifically bind to an anti-NELL1 autoantibody are conjugated to each other, e.g., are situated on one polypeptide.

In some embodiments, one or more peptides that specifically bind to an anti-NELL1 autoantibody are separated by a protease cleavage site or an IRES. In some embodiments, each of the one or more peptides is expressed as a separate peptide, e.g., translation as a separate peptide from an IRES or after cleavage of a protease cleavage site.

In some embodiments, one or more peptides that specifically bind to an anti-NELL1 autoantibody are not separated by a protease cleavage site or an IRES, e.g., is expressed as a fusion protein.

### Additional Podocyte Autoantigens:

### Neutral Endopeptidase (NEP)

Neutral endopeptidase (NEP) is a membrane-bound zinc-dependent endopeptidase and is involved in the catabolism of regulatory peptide with vasoactive properties. NEP is expressed in numerous tissues and its membrane form is naturally present on human podocytes. NEP is one of the first identified human podocyte antigens (e.g., autoantigens), which is associated with MN (e.g., neonatal alloimmune MN).

A human NEP peptide sequence is provided as SEQ ID NO: 25 (750 amino acids):

MGKSESQMDITDINTPKPKKKQRWTPLEISLSVLVLLLTIIAVTMIALYATY
DDGICKSSDCIKSAARLIQNMDATTEPCTDFFKYACGGWLKRNVIPETSSRYGNFDILRD
ELEVVLKDVLQEPKTEDIVAVQKAKALYRSCINESAIDSRGGEPLLKLLPDIYGWPVATE
NWEQKYGASWTAEKAIAQLNSKYGKKVLINLFVGTDDKNSVNHVIHIDQPRLGLPSRD
YYECTGIYKEACTAYVDFMISVARLIRQEERLPIDENQLALEMNKVMELEKEIANATAKP
EDRNDPMLLYNKMTLAQIQNNFSLEINGKPFSWLNFTNEIMSTVNISITNEEDVVVYAPE
YLTKLKPILTKYSARDLQNLMSWRFIMDLVSSLSRTYKESRNAFRKALYGTTSETATWR
RCANYVNGNMENAVGRLYVEAAFAGESKHVVEDLIAQIREVFIQTLDDLTWMDAETKK
RAEEKALAIKERIGYPDDIVSNDNKLNNEYLELNYKEDEYFENIIQNLKFSQSKQLKKLR
EKVDKDEWISGAAVVNAFYSSGRNQIVFPAGILQPPFFSAQQSNSLNYGGIGMVIGHEIT
HGFDDNGRNFNKDGDLVDWWTQQSASNFKEQSQCMVYQYGNFSWDLAGGQHLNGIN
TLGENIADNGGLGQAYRAYQNYIKKNGEEKLLPGLDLNHKQLFFLNFAQVWCGTYRPE
YAVNSIKTDVHSPGNFRIIGTLQNSAEFSEAFHCRKNSYMNPEKKCRVW

In some embodiments, a NEP polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 25. In some embodiments, a NEP polypeptide is or comprises SEQ ID NO: 25.

In some embodiments, a NEP polypeptide comprising an amino acid sequence having at least 85%, identity with SEQ ID NO:25, further comprises a different signal peptide, e.g., as disclosed herein.

In some embodiments, one or more peptides that specifically bind to an anti-NEP autoantibody comprises a fragment of a NEP polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length NEP polypeptide of SEQ ID NO: 25.

In some embodiments, one or more peptides that specifically bind to an anti-NEP autoantibody comprises a fragment of a NEP polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length NEP polypeptide of SEQ ID NO: 25.

In some embodiments, NEP is expressed in podocytes, e.g., on the membrane of podocytes. In some embodiments, a podocyte autoantigen is a NEP polypeptide or a variant or fragment thereof. In some embodiments, an anti-NEP autoantibody or a fragment or a complex thereof is characterized in that it binds to a NEP polypeptide or a variant or fragment thereof. In some embodiments, a NEP autoantigen is human NEP.

### Exostosin-1 (EXT1) and Exostosin-2 (EXT2)

Exostosin-1 (EXT1) and Exostosin-2 (EXT2) are proteins with glycosyltransferase activities and belong to the Exostosin family of proteins (Busse-Wicher M et al., Volume 35, April 2014, Pages 25-33). A subset of membranous nephropathy is associated with accumulation of EXT1 and/or EXT2 along the glomerular basement membrane. Membranous nephropathy with EXT1 and/or EXT2 accumulation is typically secondary to autoimmune disease (Sethi S et al., J Am Soc Nephrol. 2019 June; 30(6): 1123-1136).

A human EXT1 sequence is provided as SEQ ID NO: 23 (746 amino acids):
MQAKKRYFILLSAGSCLALLFYFGGLQFRASRSHSRREEHSGRNGLHHPSPDHFWPRFPD
ALRPFVPWDQLENEDSSVHISPRQKRDANSSIYKGKKCRMESCFDFTLCKKNGFKVYVY
PQQKGEKIAESYQNILAAIEGSRFYTSDPSQACLFVLSLDTLDRDQLSPQYVHNLRSKVQ
SLHLWNNGRNHLIFNLYSGTWPDYTEDVGFDIGQAMLAKASISTENFRPNFDVSIPLFSK
DHPRTGGERGFLKFNTIPPLRKYMLVFKGKRYLTGIGSDTRNALYHVHNGEDVVLLTTC
KHGKDWQKHKDSRCDRDNTEYEKYDYREMLHNATFCLVPRGRRLGSFRFLEALQAAC
VPVMLSNGWELPFSEVINWNQAAVIGDERLLLQIPSTIRSIHQDKILALRQQTQFLWEAY
FSSVEKIVLTTLEIIQDRIFKHISRNSLIWNKHPGGLFVLPQYSSYLGDFPYYYANLGLKPP
SKFTAVIHAVTPLVSQSQPVLKLLVAAAKSQYCAQIIVLWNCDKPLPAKHRWPATAVPV
VVIEGESKVMSSRFLPYDNIITDAVLSLDEDTVLSTTEVDFAFTVWQSFPERIVGYPARSH
FWDNSKERWGYTSKWTNDYSMVLTGAAIYHKYYHYLYSHYLPASLKNMVDQLANCE
DILMNFLVSAVTKLPPIKVTQKKQYKETMMGQTSRASRWADPDHFAQRQSCMNTFAS
WFGYMPLIHSQMRLDPVLFKDQVSILRKKYRDIERL
A human EXT2 sequence is provided as SEQ ID NO: 24 (718 amino acids)
MCASVKYNIRGPALIPRMKTKHRIYYITLFSIVLLGLIATGMFQFWPHSIESSNDWNVEKR
SIRDVPVVRLPADSPIPERGDLSCRMHTCFDVYRCGFNPKNKIKVYIYALKKYVDDFGVS
VSNTISREYNELLMAISDSDYYTDDINRACLFVPSIDVLNQNTLRIKETAQAMAQLSRWD
RGTNHLLFNMLPGGPPDYNTALDVPRDRALLAGGGFSTWTYRQGYDVSIPVYSPLSAEV
DLPEKGPGPRQYFLLSSQVGLHPEYREDLEALQVKHGESVLVLDKCTNLSEGVLSVRKR
CHKHQVFDYPQVLQEATFCVVLRGARLGQAVLSDVLQAGCVPVVIADSYILPFSEVLD
WKRASVVVPEEKMSDVYSILQSIPQRQIEEMQRQARWFWEAYFQSIKAIALATLQIINDR
IYPYAAISYEEWNDPPAVKWGSVSNPLFLPLIPPQSQGFTAIVLTYDRVESLFRVITEVSK
VPSLSKLLVVWNNQNKNPPEDSLWPKIRVPLKVVRTAENKLSNRFFPYDEIETEAVLAID
DDIIMLTSDELQFGYEVWREFPDRLVGYPGRLHLWDHEMNKWKYESEWTNEVSMVLT
GAAFYHKYFNYLYTYKMPGDIKNWVDAHMNCEDIAMNFLVANVTGKAVIKVTPRKKF
KCPECTAIDGLSLDQTHMVERSECINKFASVFGTMPLKVVEHRADPVLYKDDFPEKLKS
FPNIGSL

In some embodiments, EXT1 is expressed in podocytes, e.g., on the membrane of podocytes. In some embodiments, a podocyte autoantigen is an EXT1 polypeptide or a variant or fragment thereof. In some embodiments, an anti-EXT1 autoantibody or a fragment or a complex thereof is characterized in that it binds to a EXT1 polypeptide or a variant or fragment thereof. In some embodiments, a EXT1 autoantigen is human EXT1.

In some embodiments, EXT2 is expressed in podocytes, e.g., on the membrane of podocytes. In some embodiments, a podocyte autoantigen is an EXT2 polypeptide or a variant or fragment thereof. In some embodiments, an anti-EXT2 autoantibody or a fragment or a complex thereof is characterized in that it binds to a EXT2 polypeptide or a variant or fragment thereof. In some embodiments, a EXT2 autoantigen is human EXT2.

In some embodiments, a EXT1 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 23. In some embodiments, a EXT1 polypeptide is or comprises SEQ ID NO: 23.

In some embodiments, one or more peptides that specifically bind to an anti-EXT1 autoantibody comprises a fragment of an EXT1 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length EXT1 polypeptide of SEQ ID NO: 23.

In some embodiments, one or more peptides that specifically bind to an anti-EXT1 autoantibody comprises a fragment of a EXT1 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length EXT1 polypeptide of SEQ ID NO: 23.

In some embodiments, a EXT1 polypeptide comprising an amino acid sequence having at least 85%, identity with SEQ ID NO: 23, further comprises a different signal peptide, e.g., as disclosed herein.

In some embodiments, a EXT2 polypeptide comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 24. In some embodiments, a EXT2 polypeptide is or comprises SEQ ID NO: 24.

In some embodiments, one or more peptides that specifically bind to an anti-EXT2 autoantibody comprises a fragment of a EXT2 polypeptide. In some embodiments, a fragment comprises at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% of a full length EXT2 polypeptide of SEQ ID NO: 24.

In some embodiments, one or more peptides that specifically bind to an anti-EXT2 autoantibody comprises a fragment of a EXT2 polypeptide. In some embodiments, a fragment comprises no more than 95%, no more than 90%, no more than, 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, or no more than 10% of a full length EXT2 polypeptide of SEQ ID NO: 24.

In some embodiments, a EXT2 polypeptide comprising an amino acid sequence having at least 85%, identity with SEQ ID NO: 24, further comprises a different signal peptide, e.g., as disclosed herein.

### Second Moiety

A glycoengineered polypeptide disclosed herein comprises a first moiety that specifically binds to an anti-podocyte autoantibody or a fragment or a complex thereof; and a second moiety comprising one or more glycans conjugated to the first moiety at one or more glycosylation sites.

Without being bound by any particular theory, glycan engagement with endocytic carbohydrate binding proteins and receptors enables different biological pathways. These essential biological pathways are involved in modulating immune responses, mediating protein clearance, protein turnover, and controlling trafficking of soluble glycoproteins, glycolipids and any natural molecule containing a glycan moiety. The glycan-receptor interaction is determined by the glycan structure. Glycan binding receptors are highly diverse and can be exploited by glycoengineering to develop novel therapeutics based on the concept of glycan-mediated protein degradation to treat different diseases, which include but are not limited to autoimmune disorders as disclosed herein.

Further without being bound by any particular theory a glycoengineered polypeptide comprising a second moiety having one or more glycans, as described herein, is expected to activate natural degradation pathways.

In some embodiments, a second moiety of a glycoengineered polypeptide disclosed herein comprises one or more glycans and specifically binds to one or more endocytic receptors. Endocytic receptors as described herein capture glycoproteins via specific glycan structures to mediate degradation, e.g., lysosomal degradation. Endocytic receptors are ubiquitous in human and can be found on different cells.

In some embodiments, an endocytic receptor is or comprises an endocytic lectin. In some embodiments, the endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

In some embodiments, provided herein is a glycoengineered polypeptide comprising a first moiety that specifically binds to a target protein (e.g., an anti-podocyte autoantibody) and a second moiety comprising a glycan comprising terminal GlcNAc.

In some embodiments, provided herein is a glycoengineered polypeptide comprising a first moiety that specifically binds to a target protein (e.g., an anti-podocyte autoantibody) and a second moiety comprising a glycan comprising terminal GalNAc.

In some embodiments, provided herein is a glycoengineered polypeptide comprising a first moiety that specifically binds to a target protein (e.g., an anti-podocyte autoantibody) and a second moiety comprising a glycan comprising terminal Gal.

In some embodiments, a glycoengineered polypeptide provided herein can comprise (i) a binding specificity to one or more target protein(s) (e.g., one or more an anti-podocyte autoantibodies) and (ii) one or more N-glycan(s) with binding specificities to one or more endocytic receptor(s).

In some embodiments, a glycoengineered polypeptide comprises one type of N-glycan with binding specificity to one type of endocytic receptor.

In some embodiments, a glycoengineered polypeptide comprises one or more N-glycosylation sites in a first moiety. In some embodiments, one or more N-glycosylation sites in a first moiety are native N-glycosylation sites. In some embodiments, one or more N-glycosylation sites in a first moiety are engineered N-glycosylation sites. In some embodiments, a glycoengineered polypeptide comprises one or more native N-glycosylation sites and one or more engineered N-glycosylation sites.

In some embodiments, a second moiety comprising one or more glycans is conjugated, e.g., linked, to a first moiety at one or more N-glycosylation sites.

In some embodiments, a glycoengineered polypeptide comprises 1, 2, 3, 4, 5, 6, 7, 8, 9, 10 or more N-glycosylation sites (or glycosites; such as an N-glycosylation consensus sequence). These N-glycosylation sites can be glycosylated by an N-glycan such that the resulting glycoengineered bifunctional binding protein can engage with or bind to 1, 2, 3, 4, 5, 6, 7, 8, 9, 10 or more endocytic receptor molecules.

In some embodiments, a glycoengineered polypeptide comprises two types of N-glycans with binding specificities to two different endocytic receptors. In certain embodiments, a glycoengineered polypeptide provided herein can comprise 1, 2, 3, 4, 5, 6, 7, 8, 9, 10 or more polypeptide chains. Each chain can be produced in a different cell line. In certain embodiments, the glycoengineered polypeptide can be an antibody and one type of N-glycan is on the Fc domain and another type of N-glycan is on the Fab domain (eg, the variable regions) of the antibody.

In some embodiments, a glycoengineered polypeptide comprises: (i) a first type of N-glycan with binding specificity to a first endocytic receptor wherein the first type of N-glycan is present at 1, 2, 3, 4, 5, 6, 7, 8, 9, 10 or more glycosites thus engaging with or binding to 1, 2, 3, 4, 5, 6, 7, 8, 9, 10 or more molecular of the first endocytic carbohydrate-binding protein or receptor; and (ii) a second type of N-glycan with binding specificity to a second endocytic receptor wherein the second N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the second endocytic receptor(s).

In some embodiments, a glycoengineered polypeptide comprises: (i) a first type of N-glycan with binding specificity to a first endocytic receptor wherein the first type of N-glycan is present at 1, 2, 3, 4, 5, 6, 7, 8, 9, 10 or more glycosites thus engaging with or binding to 1, 2, 3, 4, 5, 6, 7, 8, 9, 10 or more molecular of the first endocytic receptor; (ii) a second type of N-glycan with binding specificity to a second endocytic receptor wherein the second N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the second endocytic receptor(s); and (iii) a third type of N-glycan with binding specificity to a third endocytic receptor wherein the third N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the third endocytic receptor(s).

In some embodiments, a glycoengineered polypeptide provided herein has 1, 2, 3, 4, 5, 6, 7, 8, 9, 10 or more glycosites. In some embodiments, in a population of glycoengineered polypeptides, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycosites in the population at one specific position are glycosylated. In certain embodiments, in a population of glycoengineered polypeptides, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycosites in the population are glycosylated. N-glycans that can be present at the glycosites of the glycoengineered polypeptide provided herein are described herein.

In some embodiments, a glycosite is an N-glycosylation consensus sequence. The consensus sequence can be N-X-S/T, or N-X-C, wherein X is any amino acid except proline.

In some embodiments, a glycosite is or comprises the sequence of GGGGANSTAPAPAPA (SEQ ID NO: 37).

In some embodiments, an N-glycan is conjugated to the glycoengineered polypeptide at at least one, two, three, or four N-glycosylation sites.

In some embodiments, an N-glycan is conjugated to the glycoengineered polypeptide at one, two, three, or four N-glycosylation sites.

In some embodiments, an N-glycosylation site is naturally occurring.

In some embodiments, an N-glycosylation site is engineered into the amino acid sequence of the first moiety.

In certain embodiments, one or more of the N-glycosylation sites are engineered into the amino acid sequence of the first moiety of the glycoengineered polypeptide (i.e. one or more of the N-glycosylation sites are not present in a wild-type, or naturally occurring form of the first moiety). In certain embodiments, at least one of the N-glycosylation sites is engineered into the amino acid sequence of the first moiety of the glycoengineered polypeptide. In certain embodiments, at least two of the N-glycosylation sites are engineered into the amino acid sequence of the first moiety of the glycoengineered polypeptide. In certain embodiments, at least three of the N-glycosylation sites are engineered into amino acid sequence of the first moiety of the glycoengineered polypeptide. In certain embodiments, at least four of the N-glycosylation sites are engineered into the amino acid sequence of the first moiety of the glycoengineered polypeptide. In certain embodiments, one or more of the engineered N-glycosylation sites are glycotags fused to the N- and/or C-terminus of the amino acid sequence of the first moiety of the glycoengineered polypeptide via a peptide linker. In certain embodiments, a glycotag is fused to the N-terminus of first moiety of the glycoengineered polypeptide. In certain embodiments, a glycotag is fused to the C-terminus of first moiety of the glycoengineered polypeptide. In certain embodiments, a glycotag is fused to the N- and the C-terminus of first moiety of the glycoengineered polypeptide. In certain embodiments, one or more of the N-glycosylation sites are natural N-glycosylation sites (i.e. one or more of the N-glycosylation sites are present in a wild-type, or naturally occurring form of the first moiety). In certain embodiments, at least one of the N-glycosylation sites is a natural N-glycosylation site. In certain embodiments, at least two of the N-glycosylation sites are natural N-glycosylation sites.

In some embodiments, provided herein is a glycoengineered polypeptide that specifically binds to a target protein associated with a disease (e.g., an anti-podocyte autoantibody), comprising a first moiety and a second moiety. In some embodiments, provided herein is a glycoengineered polypeptide comprising a first moiety that specifically binds to a target protein associated with a disease (e.g., an anti-podocyte autoantibody), and a second moiety that binds specifically to an endocytic receptor, wherein the second moiety comprises a glycan structure.

In some embodiments, provided herein is a glycoengineered polypeptide comprising a first moiety that specifically binds to a target protein and a second moiety comprising an N-glycan selected from the group consisting of GlcNAc2Man3GlcNAc2, GalNAc2GlcNAc2Man3 GlcNAc2, Gal2GlcNAc2Man3GlcNAc2, Man3 GlcNAc, GlcNAc 1Man3 GlcNAc2, Gal2GlcNAc2Man3 GlcNAc2, Gal 1 GlcNAc2Man3 GlcNAc2, GalNAc 1 GlcNAc2Man3 GlcNAc2, GlcNAc3Man3 GlcNAc2, GlcNAc4Man3 GlcNAc2, Gal3GlcNAc3Man3 GlcNAc2, GalNAc3 GlcNAc3Man3 GlcNAc2, GalNAc4GlcNAc4Man3GlcNAc2, Gal4GlcNAc4Man3GlcNAc2, or Man-6-P-N-glycan.

In some embodiments, increasing the number of glycan structures on a glycoengineered polypeptide increases the rate of lysosomal degradation as compared to an otherwise similar glycoengineered polypeptide with fewer glycan structures.

In some embodiments, the number of glycan structures on a glycoengineered polypeptide disclosed herein is 1 or more, 2 or more, 3 or more, 4 or more, 5 or more, 6 or more, 7 or more, 8 or more, 9 or more or 10 or more glycan structures.

In some embodiments, a glycoengineered polypeptide disclosed herein comprises a glycan structure having a monoantennary structure.

In some embodiments, a glycoengineered polypeptide disclosed herein comprises a glycan structure having a biantennary structure.

In some embodiments, a glycoengineered polypeptide disclosed herein comprises a glycan structure having a triantennary structure.

In some embodiments, a glycoengineered polypeptide disclosed herein comprises a glycan structure having a tetraantennary structure.

In some embodiments, the glycan structure comprises a biantennary structure. In some embodiments, the glycan structure comprises a biantennary GalNAc. In some embodiments, the biantennary GalNac binds to an asialoglycoprotein receptor (ASGPR) or a fragment or variant thereof, or a complex comprising ASGPR.

In some embodiments, the N-glycan has a structure of:

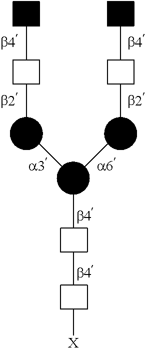

wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.

In some embodiments, the N-glycan specifically binds to one or more endocytic receptors, e.g., that mediate lysosomal degradation. In some embodiments, the N-glycan specifically binds to ASGPR.

In some embodiments, the endocytic receptor is or comprises ASGPR or a fragment or variant thereof, or a complex comprising ASGPR. In some embodiments, when the endocytic receptor is ASGPR, the glycan structure of the second moiety comprises a terminal GalNac.

ASGPR-mediated degradation in the hepatocyte has many applications. ASPGR binding to the N-glycan structure disclosed herein can result in the selective degradation of one or more target proteins (e.g., an anti-podocyte autoantibody). By way of example, ASGPR-mediated degradation can lead to removal of cytokines, chemokines and hormones. Additionally, ASGPR-mediated degradation can be used for the delivery of the target molecules to the hepatocyte endosome. Thus, ASGPR-mediated degradation is applicable for various diseases, while limiting systemic toxicity.

In certain embodiments, the 1, 2, 3, 4, 5, 6, 7, 8, 9, 10 or more N-glycosylation sites can be glycosylated by the N-glycan such that the resulting glycoengineered polypeptide can engage with or bind to 1, 2, 3, 4, 5, 6, 7, 8, 9, 10 or more endocytic receptor molecules. In certain embodiments, the glycoengineered polypeptide is glycosylated by the N-glycan at at least 2, at least 3, at least 4, at least 5, at least 6, at least 7, at least 8, at least 9, or at least 10 of the N-glycosylation sites. In certain embodiments, the glycoengineered polypeptide is glycosylated by the N-glycan at at least 2 N-glycosylation sites. In certain embodiments, the glycoengineered polypeptide is glycosylated by the N-glycan at at least 3 N-glycosylation sites. In certain embodiments, the glycoengineered polypeptide is glycosylated by the N-glycan at at least 4 N-glycosylation sites. In certain embodiments, the glycoengineered polypeptide is glycosylated by the N-glycan at at least 5 N-glycosylation sites. In certain embodiments, the glycoengineered polypeptide is glycosylated by the N-glycan at at least 6 N-glycosylation sites. In certain embodiments, the glycoengineered polypeptide is glycosylated by the N-glycan at at least 7 N-glycosylation sites. In certain embodiments, the glycoengineered polypeptide is glycosylated by the N-glycan at at least 8 N-glycosylation sites. In certain embodiments, the glycoengineered polypeptide is glycosylated by the N-glycan at at least 9 N-glycosylation sites. In certain embodiments, the glycoengineered polypeptide is glycosylated by the N-glycan at at least 10 N-glycosylation sites.

In certain embodiments, the glycoengineered polypeptide is glycosylated by the N-glycan at 2 N-glycosylation sites. In certain embodiments, the glycoengineered polypeptide is glycosylated by the N-glycan at 3 N-glycosylation sites. In certain embodiments, the glycoengineered polypeptide is glycosylated by the N-glycan at 4 N-glycosylation sites. In certain embodiments, the glycoengineered polypeptide is glycosylated by the N-glycan at 5 N-glycosylation sites. In certain embodiments, the glycoengineered polypeptide is glycosylated by the N-glycan at 6 N-glycosylation sites. In certain embodiments, the glycoengineered polypeptide is glycosylated by the N-glycan at 7 N-glycosylation sites. In certain embodiments, the glycoengineered polypeptide is glycosylated by the N-glycan at 8 N-glycosylation sites. In certain embodiments, the glycoengineered polypeptide is glycosylated by the N-glycan at 9 N-glycosylation sites. In certain embodiments, the glycoengineered polypeptide is glycosylated by the N-glycan at 10 N-glycosylation sites. In certain embodiments, the glycoengineered polypeptide is glycosylated at an Asn amino acid residue of the glycoengineered polypeptide. In certain embodiments, the N-glycosylation site is an N-glycosylation consensus sequence. In certain embodiments, the N-glycosylation site comprises a consensus sequence of N-X-S/T or N-X-C, wherein X is any amino acid except proline.

In certain embodiments, at least 10%, at least 20%, at least 30%, at least 40%, at least 50%, at least 60%, at least 70%, at least 80%, at least 90%, at least 95, or at least 98% of the N-glycosylation sites are occupied by an N-glycan. In certain embodiments, at least 10%, at least 20%, at least 30%, at least 40%, at least 50%, at least 60%, at least 70%, at least 80%, at least 90%, at least 95, or at least 98% of the N-glycosylation sites are occupied by an N-glycan of the structure:

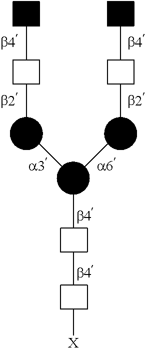

linked to the glycoengineered polypeptide at one or more N-glycosylation sites, wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide. In certain embodiments, at least 10% of the N-glycosylation sites are occupied by the N-glycan. In certain embodiments, at least 20% of the N-glycosylation sites are occupied by the N-glycan. In certain embodiments, at least 30% of the N-glycosylation sites are occupied by the N-glycan. In certain embodiments, at least 40% of the N-glycosylation sites are occupied by the N-glycan. In certain embodiments, at least 50% of the N-glycosylation sites are occupied by the N-glycan. In certain embodiments, at least 60% of the N-glycosylation sites are occupied by the N-glycan. In certain embodiments, at least 70% of the N-glycosylation sites are occupied by the N-glycan. In certain embodiments, at least 80% of the N-glycosylation sites are occupied by the N-glycan. In certain embodiments, at least 90% of the N-glycosylation sites are occupied by the N-glycan. In certain embodiments, at least 95% of the N-glycosylation sites are occupied by the N-glycan. In certain embodiments, at least 98% of the N-glycosylation sites are occupied by the N-glycan.

In certain embodiments, the N-glycan is linked to the glycoengineered polypeptide at at least one N-glycosylation site. In certain embodiments, the N-glycan is linked to the glycoengineered polypeptide at at least two N-glycosylation sites. In certain embodiments, the N-glycan is linked to the glycoengineered polypeptide at one, two, three, or four N-glycosylation sites. In certain embodiments, the N-glycan is linked to the glycoengineered polypeptide at one N-glycosylation site. In certain embodiments, the N-glycan is linked to the glycoengineered polypeptide at two N-glycosylation sites. In certain embodiments, the N-glycan is linked to the glycoengineered polypeptide at three N-glycosylation sites. In certain embodiments, the N-glycan is linked to the glycoengineered polypeptide at four N-glycosylation sites. In certain embodiments, the N-glycan is linked to the glycoengineered polypeptide at an Asn amino acid residue of the glycoengineered polypeptide. In certain embodiments, the N-glycan is linked to the glycoengineered polypeptide at an N-glycosylation consensus sequence. In certain embodiments, the N-glycan is linked to the glycoengineered polypeptide at a consensus sequence of N-X-S/T or N-X-C, wherein X is any amino acid except proline.

In certain embodiments, the glycoengineered polypeptide comprises two different N-glycans (i.e. a first and a second N-glycan), wherein each N-glycan is independently linked to the glycoengineered polypeptide at 1, 2, 3, 4, 5, 6, 7, 8, 9, 10 or more N-glycosylation sites, and wherein one of the N-glycans (i.e. the first N-glycan) has the structure:

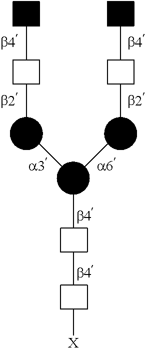

wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide. In certain embodiments, the different N-glycans specifically bind to different endocytic receptors. In certain embodiments, the first N-glycan specifically binds to ASGPR. In certain embodiments, the other N-glycan is an N-glycan described in PCT/EP2022/057556, which is incorporated herein by reference in its entirety. In certain embodiments, the first N-glycan is larger than the second N-glycan. In other embodiments, the first N-glycan is smaller than the second N-glycan. In certain embodiments, the N-glycosylation sites predominantly or exclusively occupied by the larger N-glycan are more sterically accessible than the N-glycosylation sites predominantly or exclusively occupied by the smaller N-glycan. In certain embodiments, the other N-glycan is A2. In certain embodiments, the other N-glycan is A1GalNAc1 or A2GalNAc1. In certain embodiments, the N-glycans are linked to the glycoengineered polypeptide at an Asn amino acid residue of the glycoengineered polypeptide. In certain embodiments, the N-glycans are linked to the glycoengineered polypeptide at an N-glycosylation consensus sequence. In certain embodiments, the N-glycans are linked to the glycoengineered polypeptide at a consensus sequence of N-X-S/T or N-X-C, wherein X is any amino acid except proline. In certain embodiments, the first N-glycan is linked to the glycoengineered polypeptide at 1, 2, 3, 4, 5, 6, 7, 8, 9, 10 or more N-glycosylation sites, and the second N-glycan is linked to the glycoengineered polypeptide at 1, 2, 3, 4, 5, 6, 7, 8, 9, 10 or more N-glycosylation sites.

In certain embodiments, the glycoengineered polypeptide further comprises a third N-glycan, wherein the third N-glycan is linked to the glycoengineered polypeptide at 1, 2, 3, 4, 5, 6, 7, 8, 9, 10 or more N-glycosylation sites. In certain embodiments, the third N-glycan specifically binds to a different endocytic receptor than the first and/or second N-glycan. In certain embodiments, the third N-glycan is an N-glycan described in PCT/EP2022/057556, which is incorporated herein by reference in its entirety. In certain embodiments, the third N-glycan is A2. In certain embodiments, the third N-glycan is A1GalNAc1 or A2GalNAc1. In certain embodiments, the third N-glycan is linked to the glycoengineered polypeptide at an Asn amino acid residue of the glycoengineered polypeptide. In certain embodiments, the third N-glycan is linked to the glycoengineered polypeptide at an N-glycosylation consensus sequence. In certain embodiments, the third N-glycan is linked to the glycoengineered polypeptide at a consensus sequence of N-X-S/T or N-X-C, wherein X is any amino acid except proline.

In certain embodiments, the second and/or third N-glycan specifically bind to an endocytic lectin. In some embodiments, the endocytic lectin is a mannose binding receptor. In some embodiments, the endocytic lectin is a Cluster of Differentiation 206 (CD206) receptor. In some embodiments, the endocytic lectin is a DC-SIGN (Cluster of Differentiation 209 or CD209) receptor. In some embodiments, the endocytic lectin is a C-Type Lectin Domain Family 4 Member G (LSECTin) receptor. In some embodiments, the endocytic lectin is a macrophage inducible Ca²⁺-dependent lectin receptor (Mincle). In some embodiments, the endocytic receptor is L-SIGN CD209L. In some embodiments, the endocytic receptor is asialoglycoprotein (ASGPR). In some embodiments, the endocytic receptor is dectin-1. In some embodiments, the endocytic receptor is dectin-2. In some embodiments, the endocytic receptor is langerin. In some embodiments, the second and/or third N-glycan specifically bind to a receptor selected from the group consisting of macrophage mannose 2 receptor, BDCA-2, DCIR, MBL, MDL, MICL, CLEC2, CLEC10, DNGR1, CLEC12B, DEC-205, and mannose 6 phosphate receptor (M6PR).

CD206 is a C-type lectin and phagocytic/endocytic recycling and signaling receptor. CD206 is expressed primarily by M2 anti-inflammatory macrophages, dendritic cells, and live sinusoidal endothelial cells. DC-SIGN is a non-recycling, signaling receptor that targets both the ligand and receptor to the lysosome for degradation. LSECTin is expressed on liver sinusoidal endothelial cells.

In certain embodiments, the glycoengineered polypeptide is glycosylated at two or more N-glycosylation sites by an N-glycan of the structure:

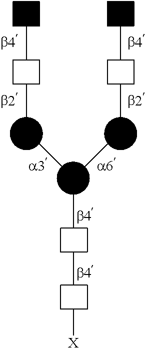

wherein the black square represents an N-acetyl galactosamine (GalNAc), the white square represents an N-acetylglucosamine (GlcNAc) residue the black circle represents a mannose (Man) residue, and X represents an amino acid residue of the glycoengineered polypeptide, and wherein two of the N-glycosylation sites are separated by at least 5, at least 10, at least 20, at least 30, at least 40, at least 50, at least 60, at least 70, at least 80, at least 90, or at least 100 amino acids. In certain embodiments, the N-glycan is linked to the glycoengineered polypeptide at two N-glycosylation sites separated by a distance of about 5-10, about 10-20, about 20-30, about 30-40, about 40-50, about 50-60, about 60-70, about 70-80, about 80-90, about 90-100, about 100-150, about 150-200, or about 200-300 amino acids. In certain embodiments, the amino acid separation between the N-glycosylation sites is the number of amino acids between the terminal amino acids of the N-glycosylation consensus sequence. Without being bound by theory, the glycoengineered polypeptide folds in space and, thus, has a three-dimensional geometry in addition to its primary amino acid structure. Also without being bound by theory, this three-dimensional geometry, including the position of the N-glycan is not static but dynamic (see, for example, Re, S., et al Biophysical Reviews, 4, 179-187 (2012)). Notwithstanding, in certain embodiments, the distance between N-glycosylation sites and/or N-glycans on a glycoengineered polypeptide may be from an equilibrium geometry of the glycoengineered polypeptide, as determined by any standard means known in the art, including for example computational modelling studies. In certain embodiments, the N-glycan is linked to the glycoengineered polypeptide at two N-glycosylation sites separated by a distance of at least 1.0 nm. In certain embodiments, the N-glycan is linked to the glycoengineered polypeptide at two N-glycosylation sites separated by a distance of about 1.0-5.0 nm. In certain embodiments, the N-glycan is linked to the glycoengineered polypeptide at two N-glycosylation sites separated by a distance of about 1.5-3.0 nm. In certain embodiments, the N-glycan is linked to the glycoengineered polypeptide at two N-glycosylation sites separated by a distance of about 1.5-2.5 nm. In certain embodiments, the N-glycan is linked to the glycoengineered polypeptide at three N-glycosylation sites each separated by a distance of about 1.0-5.0 nm. In certain embodiments, the N-glycan is linked to the glycoengineered polypeptide at three N-glycosylation sites each separated by a distance of about 1.5-3.0 nm. In certain embodiments, the N-glycan is linked to the glycoengineered polypeptide at three N-glycosylation sites each separated by a distance of about 1.5-2.5 nm. In certain embodiments, the N-glycans are separated by a distance of at least 1.0 nm. In certain embodiments, the N-glycans are separated by a distance of about 1.0 to about 5.0 nm. In certain embodiments, the N-glycans are separated by a distance of about 1.5 to about 2.5 nm. In certain embodiments, the distance between the N-glycosylation sites and/or N-glycans is chosen to minimize steric hindrance, for example between the glycoengineered polypeptide (s), the target protein(s), and/or the ASGPR receptor(s). In certain embodiments, the distance between the N-glycosylation sites and/or N-glycans is chosen based on the separation of ASGPR receptors on a cell surface. In certain embodiments, the distance between the N-glycosylation sites and/or N-glycans is chosen to be similar (e.g. no more than twice, or no less than half) to the separation of ASGPR receptors on a cell surface. In certain embodiments, the N-glycan is linked to the glycoengineered polypeptide at an Asn amino acid residue of the glycoengineered polypeptide. In certain embodiments, the N-glycan is linked to the glycoengineered polypeptide at an N-glycosylation consensus sequence. In certain embodiments, the N-glycan is linked to the glycoengineered polypeptide at a consensus sequence of N-X-S/T or N-X-C, wherein X is any amino acid except proline.

### Nucleic Acid Sequences Encoding Glycoengineered Polypeptides

The present disclosure, among other things, provides nucleic acids encoding glycoengineered polypeptides as described herein.

In some embodiments, a nucleic acid is or comprises single stranded DNA (e.g., as in certain viral vectors). In some embodiments, a nucleic acid is or comprises double stranded DNA (e.g., as in certain viral vectors and/or certain plasmids). In some embodiments, a nucleic acid is or comprises RNA (e.g., as in certain viral vectors and/or as in mRNA therapeutics), etc.

Nucleic acids encoding glycoengineered polypeptides may be modified to include codons that are optimized for expression in a particular cell type (e.g., a Leishmania cell) or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acids encoding glycoengineered polypeptides described herein, in whole or in part, may include an altered sequence to optimize codon usage for a particular cell type (e.g., a eukaryotic or prokaryotic cell). For example, a coding sequence for an antibody agent (e.g., antigen binding fragment) as described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host (e.g., Leishmania host cell). Selection of vectors and methods to construct them are commonly known to persons of ordinary skill in the art.

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding a glycoengineered polypeptide described herein under control of transcriptional and/or translational control signals. These methods may include in vitro recombinant DNA and synthetic techniques and in vivo recombination (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

### Compositions and Pharmaceutical Compositions

A composition disclosed herein may comprise and/or deliver one or more glycoengineered polypeptides disclosed herein or nucleic acids encoding one or more glycoengineered polypeptides disclosed herein.

In some embodiments, a composition disclosed herein comprises a glycoengineered polypeptide comprising a first moiety and a second moiety. In some embodiments, a composition disclosed herein comprises a plurality of glycoengineered polypeptides comprising a first moiety and a second moiety.

In some embodiments, a composition comprising a plurality of glycoengineered polypeptides comprises 1, 2, 3, 4, 5, or more glycoengineered polypeptides comprising a first moiety that binds to an anti-podocyte autoantibody (e.g., a first moiety that binds to the same anti-podocyte autoantibody).

In some embodiments, a composition comprising a plurality of glycoengineered polypeptides comprises glycoengineered polypeptides having a first moiety that binds to an anti-PLA2R autoantibody or a fragment thereof. In some embodiments, the glycoengineered polypeptides in the plurality each comprise the same first moiety. In some embodiments, the glycoengineered polypeptides in the plurality each comprise a different first moiety (e.g., a first glycoengineered polypeptide comprises a first moiety that binds to an anti-PLA2R autoantibody or a fragment thereof, a second glycoengineered polypeptide comprises a different first moiety that binds to an anti-PLA2R autoantibody or a fragment thereof, etc.).

In some embodiments, a composition comprising a plurality of glycoengineered polypeptides comprises glycoengineered polypeptides having a first moiety that binds to an anti-THSD7A autoantibody or a fragment thereof. In some embodiments, the glycoengineered polypeptides in the plurality each comprise the same first moiety. In some embodiments, the glycoengineered polypeptides in the plurality each comprise a different first moiety (e.g., a first glycoengineered polypeptide comprises a first moiety that binds to an anti-THSD7A autoantibody or a fragment thereof, a second glycoengineered polypeptide comprises a different first moiety that binds to an anti-THSD7A autoantibody or a fragment thereof, etc.).

In some embodiments, a composition comprising a plurality of glycoengineered polypeptides comprises glycoengineered polypeptides having a first moiety that binds to an anti-NELL1 autoantibody or a fragment thereof. In some embodiments, the glycoengineered polypeptides in the plurality each comprise the same first moiety. In some embodiments, the glycoengineered polypeptides in the plurality each comprise a different first moiety (e.g., a first glycoengineered polypeptide comprises a first moiety that binds to an anti-NELL1 autoantibody or a fragment thereof, a second glycoengineered polypeptide comprises a different first moiety that binds to an anti-NELL1 autoantibody or a fragment thereof, etc.).

In some embodiments, a composition comprising a plurality of glycoengineered polypeptides comprises 1, 2, 3, 4, 5, or more glycoengineered polypeptides each comprising a first moiety that binds to a different anti-podocyte autoantibody (e.g., an anti-PLA2R autoantibody or a fragment thereof, or an anti-THSD7A autoantibody or a fragment thereof or an anti-NELL1 autoantibody or a fragment thereof. In some embodiments, a composition comprising a plurality of glycoengineered polypeptides comprises a first glycoengineered polypeptide comprising a first moiety that binds to an anti-podocyte autoantibody (e.g., an anti-PLA2R autoantibody or a fragment thereof), a second glycoengineered polypeptide comprising a first moiety that binds to a anti-podocyte autoantibody (e.g., an anti-THSD7A autoantibody or a fragment thereof) and a third glycoengineered polypeptide comprising a first moiety that binds to a different anti-podocyte autoantibody (e.g., an anti-NELL1 autoantibody or a fragment thereof).

In some embodiments, a glycoengineered polypeptide disclosed herein (i) a first glycoengineered polypeptide comprising a first moiety that specifically binds to an anti-PLA2R autoantibody or a fragment or a complex thereof, (ii) a second glycoengineered polypeptide comprising a first moiety that specifically binds to an anti-THSD7A autoantibody or a fragment or a complex thereof; and (iii) a third glycoengineered polypeptide comprising a first moiety that specifically binds to an anti-NELL1 autoantibody or a fragment or a complex thereof

In some embodiments, ratio of the first glycoengineered polypeptide to the second glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

In some embodiments, the ratio of the first glycoengineered polypeptide to the second glycoengineered polypeptide is about 1:5 to about 5:1; about 1:2.5 to about 2.5:1.

In some embodiments, the ratio of the first glycoengineered polypeptide to the second glycoengineered polypeptide is about 1:1.5 to about 1.5:1.

In some embodiments, ratio of the first glycoengineered polypeptide to the third glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

In some embodiments, the ratio of the first glycoengineered polypeptide to the third glycoengineered polypeptide is about 1:5 to about 5:1; about 1:2.5 to about 2.5:1.

In some embodiments, the ratio of the first glycoengineered polypeptide to the third glycoengineered polypeptide is about 1:1.5 to about 1.5:1.

In some embodiments, ratio of the second glycoengineered polypeptide to the third glycoengineered polypeptide is about 1:9, about 1:8, about 1:7, about 1:6, about 1:5, about 1:4, about 1:3, about 1:2.5, about 1:2, about 1:1.5, about 1:1, about 9:1, about 8:1, about 7:1, about 6:1, about 5:1, about 4:1, about 3:1, about 2.5:1 about, 2:1, or about 1.5:1.

In some embodiments, the ratio of the second glycoengineered polypeptide to the third glycoengineered polypeptide is about 1:5 to about 5:1; about 1:2.5 to about 2.5:1.

In some embodiments, the ratio of the second glycoengineered polypeptide to the third glycoengineered polypeptide is about 1:1.5 to about 1.5:1.

In some embodiments, the first glycoengineered polypeptide is present at an amount of about 10-90% and the additional (e.g., second or third) glycoengineered polypeptide is present at an amount of about 90-10%.

In some embodiments, the first glycoengineered polypeptide is present at an amount of about 20-80% and the additional (e.g., second or third) glycoengineered polypeptide is present at an amount of about 80-20%.

In some embodiments, the first glycoengineered polypeptide is present at an amount of about 30-70% and the additional (e.g., second or third) glycoengineered polypeptide is present at an amount of about 70-30%.

In some embodiments, the first glycoengineered polypeptide is present at an amount of about 40-60% and the additional (e.g., second or third) glycoengineered polypeptide is present at an amount of about 60-40%.

In some embodiments, the first glycoengineered polypeptide is present at an amount of about 10% and the additional (e.g., second or third) glycoengineered polypeptide is present at an amount of about 90%.

In some embodiments, the first glycoengineered polypeptide is present at an amount of about 20% and the additional (e.g., second or third) glycoengineered polypeptide is present at an amount of about 80%.

In some embodiments, the first glycoengineered polypeptide is present at an amount of about 30% and the additional (e.g., second or third) glycoengineered polypeptide is present at an amount of about 70%.

In some embodiments, the first glycoengineered polypeptide is present at an amount of about 40% and the additional (e.g., second or third) glycoengineered polypeptide is present at an amount of about 60%.

In some embodiments, the first glycoengineered polypeptide is present at an amount of about 50% and the additional (e.g., second or third) glycoengineered polypeptide is present at an amount of about 50%.

In some embodiments, the first glycoengineered polypeptide is present at an amount of about 60% and the additional (e.g., second or third) glycoengineered polypeptide is present at an amount of about 40%.

In some embodiments, the first glycoengineered polypeptide is present at an amount of about 70% and the additional (e.g., second or third) glycoengineered polypeptide is present at an amount of about 30%.

In some embodiments, the first glycoengineered polypeptide is present at an amount of about 80% and the additional (e.g., second or third) glycoengineered polypeptide is present at an amount of about 20%.

In some embodiments, the first glycoengineered polypeptide is present at an amount of about 90% and the additional (e.g., second or third) glycoengineered polypeptide is present at an amount of about 10%.

In some embodiments, disclosed herein is a composition comprising a population of glycoengineered polypeptides disclosed herein, wherein the population of glycoengineered polypeptides has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s).

In some embodiments, the homogeneity of the N-glycan profile at one or more of the N-glycosylation sites is determined by N-glycan analysis, glycopeptide analysis or intact protein analysis.

In some embodiments, the N-glycan profile comprises about 30% to 40%, about 40% to about 50%, about 50% to about 60%, about 60% to about 70%, about 70% to about 80%, about 80% to about 90%, or about 90% to about 100% of the N-glycan of the structure provided herein.

In some embodiments, the population of glycoengineered polypeptides has an N-glycan profile comprising about 30% to about 40%, about 40% to about 50%, about 50% to about 60%, about 60% to about 70%, about 70% to about 80%, about 80% to about 90%, or about 90% to about 100% of the N-glycan of the structure provided herein among all glycans in the N-glycan profile.

In some embodiments, a glycoengineered polypeptide disclosed herein or a composition comprising the same may be useful to treat and/or prevent a disease described herein (e.g., a disease associated anti-podocyte autoantibodies), or to ameliorate a symptom associated with a disease, disorder or condition described herein.

The present disclosure also provides pharmaceutical compositions that, when administered to a subject (e.g., a human subject), e.g., when administered to a subject suffering from a disease associated with anti-podocyte autoantibodies, deliver a glycoengineered polypeptide as described herein to such subject. Thus, in some embodiments, the present disclosure provides pharmaceutical compositions that comprise or deliver one or more glycoengineered polypeptides or one or more polynucleotides encoding such as described herein.

In some embodiments, a pharmaceutical composition is or comprises a composition according to the present disclosure.

Typically, a pharmaceutical composition includes an glycoengineered polypeptide as described herein, or a nucleic acid that encodes it in combination with one or more pharmaceutically acceptable carriers or excipients such as, for example one or more buffers, diluents, fillers, salts, solubilizers, stabilizers, and/or other materials as is known in the art. Those skilled in the art will be aware of a variety of carrier components appropriate to a particular active type (e.g., polypeptide versus nucleic acid, viral vector vs plasmid versus RNA, etc.) and/or route of administration (e.g., parenteral, enteral, etc.).

In some embodiments, a pharmaceutical composition, may comprise or deliver two or more different glycoengineered polypeptide, so that such agents may be administered in combination (e.g., substantially simultaneously or sequentially) to subject(s).

In some embodiments, a pharmaceutical composition may contain one or more agents that, for example, may improve stability of the composition and/or its active agent (e.g., to particular storage conditions and/or period(s) of time), facilitate delivery of the composition and/or its active agent, and/or otherwise enhance effectiveness (and/or reduce one or more undesirable side effects) of the active agent or composition once administered.

Alternatively or additionally, in some embodiments, a provided pharmaceutical composition may comprise or deliver another active agent in addition to an glycoengineered polypeptide as described herein.

Methods of Treatment and/or Prevention

The present disclosure, among other things, provides methods of treating and/or preventing membranous nephropathy (e.g., iMN) in a subject comprising administering a composition as described herein, thereby improving at least one sign or symptom of membranous nephropathy (e.g., iMN) in the subject after administration.

In some embodiments, provided herein are methods of treating and/or preventing a membranous nephropathy (e.g., iMN), comprising administering a composition as described herein.

Among other things, disclosed herein is the identification of glycoengineered polypeptides that specially bind to anti-podocyte autoantibodies or fragments or complexes thereof (e.g., anti-PLA2R autoantibodies, anti-NELL1 autoantibodies and/or anti-THSD7A autoantibodies). In some embodiments, glycoengineered polypeptides disclosed herein have therapeutic value, e.g., in the treatment of membranous nephropathy (e.g., iMN).

A glycoengineered polypeptide that specifically binds to an anti-podocyte autoantibody (e.g., anti-PLA2R autoantibodies, anti-NELL1 autoantibodies and/or anti-THSD7A autoantibodies) of the present disclosure can be used, inter alia, to treat, prevent, and/or improve membranous nephropathy (e.g., iMN).

Additionally or alternatively, a glycoengineered polypeptide that specifically binds to an anti-podocyte autoantibody (e.g., anti-PLA2R autoantibodies, anti-NELL1 autoantibodies and/or anti-THSD7A autoantibodies) of the present disclosure can be used, inter alia, to treat, prevent, and/or improve, any number of diseases in which the anti-podocyte autoantibody levels are aberrantly high and/or in which a reduction of anti-podocyte autoantibody levels is sought.

A subject to be treated with methods described herein can be e.g., a patient having, or at risk of having, or is diagnosed as having membranous nephropathy (e.g., iMN).

Those skilled in the art, reading the present disclosure, will appreciate that provided compositions may be useful for treating membranous nephropathy (e.g., iMN). In some embodiments, a subject having membranous nephropathy (e.g., iMN) has or is characterized as having increased levels of anti-podocyte autoantibodies. In some embodiments, a subject having membranous nephropathy (e.g., iMN) has or is characterized as having aberrant anti-podocyte autoantibodies.

In some embodiment, a subject has an anti-PLA2R autoantibody. In some embodiments, administration of a composition reduces a level of an anti-PLA2R autoantibody or a complex comprising the same as compared to a subject who has not been administered the composition or as compared to the same subject prior to administration of the composition.

In some embodiment, a subject has an anti-PLA2R autoantibody. In some embodiments, administration of a composition reduces and/or prevents formation and/or accumulation of an immune complex as compared to a subject who has not been administered the pharmaceutical composition or as compared to the same subject prior to administration of the pharmaceutical composition. In some embodiments, the immune complex comprises one or more: IgG antibodies, the target, an antigen recognized by the target, complement components, or combinations thereof. In some embodiments, the immune complex comprising one or more complement components comprises a membrane attack complex. In some embodiments, the one or more complement components comprise: C3, C5b, C6, C7, C8, and/or C9.

In some embodiment, a subject has an anti-PLA2R autoantibody. In some embodiments, administration of a composition reduces activation of a complement pathway or component thereof as compared to a subject who has not been administered the pharmaceutical composition or as compared to the same subject prior to administration of the pharmaceutical composition. In some embodiments, a complement pathway comprises: a lectin pathway, a classical pathway or an alternative pathway.

In some embodiment, a subject has an anti-PLA2R autoantibody. In some embodiments, administration of a composition reduces and/or prevents proteinuria, decreased albumin levels and/or edema as compared to a subject who has not been administered the pharmaceutical composition or as compared to the same subject prior to administration of the pharmaceutical composition.

In some embodiment, a subject has an anti-PLA2R autoantibody. In some embodiments, administration of a composition reduces and/or prevents thickening of glomerular capillary walls as compared to a subject who has not been administered the pharmaceutical composition or as compared to the same subject prior to administration of the pharmaceutical composition.

In some embodiment, a subject has an anti-PLA2R autoantibody. In some embodiments, administration of a composition reduces and/or prevents changes in structure and/or function of podocytes as compared to a subject who has not been administered the pharmaceutical composition or as compared to the same subject prior to administration of the pharmaceutical composition. In some embodiments, administration of the pharmaceutical composition prevents apoptosis in podocytes. In some embodiments, administration of the pharmaceutical composition prevents an increase in intracellular calcium in human podocytes. In some embodiments, administration of the pharmaceutical composition prevents a decrease in cellular motility and/or adhesion of podocytes.

In some embodiment, a subject has an anti-THSD7A autoantibody. In some embodiments, administration of a composition reduces a level of an anti-THSD7A autoantibody or a complex comprising the same as compared to a subject who has not been administered the composition or as compared to the same subject prior to administration of the composition.

In some embodiment, a subject has an anti-THSD7A autoantibody. In some embodiments, administration of a composition reduces and/or prevents formation and/or accumulation of an immune complex as compared to a subject who has not been administered the pharmaceutical composition or as compared to the same subject prior to administration of the pharmaceutical composition. In some embodiments, the immune complex comprises one or more: IgG antibodies, the target, an antigen recognized by the target, complement components, or combinations thereof. In some embodiments, the immune complex comprising one or more complement components comprises a membrane attack complex. In some embodiments, the one or more complement components comprise: C3, C5b, C6, C7, C8, and/or C9.

In some embodiment, a subject has an anti-THSD7A autoantibody. In some embodiments, administration of a composition reduces activation of a complement pathway or component thereof as compared to a subject who has not been administered the pharmaceutical composition or as compared to the same subject prior to administration of the pharmaceutical composition. In some embodiments, a complement pathway comprises: a lectin pathway, a classical pathway or an alternative pathway.

In some embodiment, a subject has an anti-THSD7A autoantibody. In some embodiments, administration of a composition reduces and/or prevents proteinuria, decreased albumin levels and/or edema as compared to a subject who has not been administered the pharmaceutical composition or as compared to the same subject prior to administration of the pharmaceutical composition.

In some embodiment, a subject has an anti-THSD7A autoantibody. In some embodiments, administration of a composition reduces and/or prevents thickening of glomerular capillary walls as compared to a subject who has not been administered the pharmaceutical composition or as compared to the same subject prior to administration of the pharmaceutical composition.

In some embodiment, a subject has an anti-THSD7A autoantibody. In some embodiments, administration of a composition reduces and/or prevents changes in structure and/or function of podocytes as compared to a subject who has not been administered the pharmaceutical composition or as compared to the same subject prior to administration of the pharmaceutical composition. In some embodiments, administration of the pharmaceutical composition prevents apoptosis in podocytes. In some embodiments, administration of the pharmaceutical composition prevents an increase in intracellular calcium in human podocytes. In some embodiments, administration of the pharmaceutical composition prevents a decrease in cellular motility and/or adhesion of podocytes.

In some embodiment, a subject has an anti-NELL1 autoantibody. In some embodiments, administration of a composition reduces a level of an anti-NELL1 autoantibody or a complex comprising the same as compared to a subject who has not been administered the composition or as compared to the same subject prior to administration of the composition.

In some embodiment, a subject has an anti-NELL1 autoantibody. In some embodiments, administration of a composition reduces and/or prevents formation and/or accumulation of an immune complex as compared to a subject who has not been administered the pharmaceutical composition or as compared to the same subject prior to administration of the pharmaceutical composition. In some embodiments, the immune complex comprises one or more: IgG antibodies, the target, an antigen recognized by the target, complement components, or combinations thereof. In some embodiments, the immune complex comprising one or more complement components comprises a membrane attack complex. In some embodiments, the one or more complement components comprise: C3, C5b, C6, C7, C8, and/or C9.

In some embodiment, a subject has an anti-NELL1 autoantibody. In some embodiments, administration of a composition reduces activation of a complement pathway or component thereof as compared to a subject who has not been administered the pharmaceutical composition or as compared to the same subject prior to administration of the pharmaceutical composition. In some embodiments, a complement pathway comprises: a lectin pathway, a classical pathway or an alternative pathway.

In some embodiment, a subject has an anti-NELL1 autoantibody. In some embodiments, administration of a composition reduces and/or prevents proteinuria, decreased albumin levels and/or edema as compared to a subject who has not been administered the pharmaceutical composition or as compared to the same subject prior to administration of the pharmaceutical composition.

In some embodiment, a subject has an anti-NELL1 autoantibody. In some embodiments, administration of a composition reduces and/or prevents thickening of glomerular capillary walls as compared to a subject who has not been administered the pharmaceutical composition or as compared to the same subject prior to administration of the pharmaceutical composition.

In some embodiment, a subject has an anti-NELL1 autoantibody. In some embodiments, administration of a composition reduces and/or prevents changes in structure and/or function of podocytes as compared to a subject who has not been administered the pharmaceutical composition or as compared to the same subject prior to administration of the pharmaceutical composition. In some embodiments, administration of the pharmaceutical composition prevents apoptosis in podocytes. In some embodiments, administration of the pharmaceutical composition prevents an increase in intracellular calcium in human podocytes. In some embodiments, administration of the pharmaceutical composition prevents a decrease in cellular motility and/or adhesion of podocytes.

Also disclosed herein is a method comprising, assessing a level of an anti-podocyte autoantibody in a sample from a subject, and administering a pharmaceutical composition disclosed herein, if the level of the anti-podocyte autoantibody is higher than a comparator. In some embodiments, a comparator comprises a predetermined reference sample such as a sample obtained from an otherwise similar subject who does not have a disease or disorder, or a symptom of a disease or disorder.

In some embodiments, an anti-podocyte autoantibody comprises: an anti-PLA2R autoantibody or a fragment or a complex thereof; an anti-NELL1 autoantibody or a fragment or a complex thereof; or an anti-THSD7A autoantibody or a fragment or a complex thereof, or a combination thereof.

### Administration

In some embodiments of the disclosure, provided are methods comprising administering to a subject a composition according to the present disclosure. In some embodiments, a method comprises administering to a subject a pharmaceutical composition comprising a glycoengineered polypeptide according to the present disclosure.

Delivery of a glycoengineered polypeptide can be achieved e.g., by administration of a pharmaceutical composition as described herein, such as a pharmaceutical composition that comprises a glycoengineered polypeptide or a nucleic acid that encodes it, for example via oral ingestion, inhalation, topical application or parenteral administration (e.g., cutaneous, subcutaneous, intraperitoneal, intramuscular or intravenous injection). In some embodiments, administration is by intravenous or intramuscular injection. In some embodiments, local administration may be or comprise topical administration (e.g., to the skin) or parenteral administration (e.g., by injection to a site of deposition such as to the kidney).

In some embodiments, delivery of a glycoengineered polypeptide can be achieved e.g., by administration of a pharmaceutical composition as described herein, such as a pharmaceutical composition that comprises a glycoengineered polypeptide or a nucleic acid that encodes it, may be oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural). Those skilled in the art will be aware of typical guiding principles for formulation of pharmaceutical compositions for administration by such routes. For example, such techniques may include the step of bringing into association a glycoengineered polypeptide or a nucleic acid that encodes it and the pharmaceutical carrier(s) or excipient(s). In some embodiments, compositions are prepared by uniformly and intimately bringing into association the active ingredient with liquid carriers or finely divided solid carriers or both, and then, if necessary, shaping the product.

In some embodiments, the administration step comprises intravenous injection, intraperitoneal injection, subcutaneous injection, transdermal injection, or intramuscular injection.

In some embodiments, a composition according to the present disclosure are delivered to a subject suffering from or susceptible to membranous nephropathy (e.g., iMN).

In some embodiments, the subject has or is diagnosed as having membranous nephropathy (e.g., iMN). In some embodiments, the individual is a human.

In some embodiments, a subject has or is diagnosed as having anti-PLA2R autoantibodies, anti-NELL1 autoantibodies, anti-THSD7A autoantibodies, or other anti-podocyte autoantibodies disclosed herein. In some embodiments, a subject having anti-PLA2R autoantibodies is treated with a glycoengineered polypeptide or a nucleic acid that encodes it that specifically binds anti-PLA2R autoantibodies. In some embodiments, a subject having anti-NELL1 autoantibodies is treated with a glycoengineered polypeptide or a nucleic acid that encodes it that specifically binds anti-NELL1 autoantibodies. In some embodiments, a subject having anti-THSD7A autoantibodies is treated with a glycoengineered polypeptide or a nucleic acid that encodes it that specifically binds anti-THSD7A autoantibodies.

In some embodiments, administration of a composition according to the present disclosure alleviates one or more symptoms of membranous nephropathy (e.g., iMN).

In some embodiments, administration of a glycoengineered polypeptide that binds to an anti-PLA2R autoantibody treats and/or prevents membranous nephropathy (e.g., iMN).

In some embodiments, administration of a glycoengineered polypeptide that binds to an anti-THSD7A autoantibody treats and/or prevents membranous nephropathy (e.g., iMN).

In some embodiments, administration of a glycoengineered polypeptide that binds to an anti-NELL1 autoantibody treats and/or prevents membranous nephropathy (e.g., iMN).

In some embodiments, administration of a glycoengineered polypeptide that binds to an anti-EXT1 autoantibody treats and/or prevents membranous nephropathy (e.g., iMN).

In some embodiments, administration of a glycoengineered polypeptide that binds to an anti-EXT2 autoantibody treats and/or prevents membranous nephropathy (e.g., iMN).

### Dosing Regimens

In some embodiments, a method comprises administering a composition once. In some embodiments, a method comprises administering a composition repeatedly.

In some embodiments, administration of a composition is continued to maintain remission (e.g., keep anti-podocyte autoantibodies low and/or undetectable) and/or avoid relapse.

Amounts glycoengineered polypeptide administered in a single dose may depend on the nature and/or severity of the condition being treated and/or on the nature of prior treatments that the patient has undergone. In some embodiments, the attending physician decides the amount of glycoengineered polypeptide with which to treat each individual patient. In some embodiments, the attending physician initially administers low doses of glycoengineered polypeptides of the present invention and observe the patient's response. In some embodiments, larger doses are administered until an optimal therapeutic effect is obtained for the patient, after which dosage is not increased further.

In some embodiments, a glycoengineered polypeptide according to the present disclosure is delivered in an amount effective to reduce levels of anti-podocyte autoantibody or a fragment or an immune complex comprising the same.

In some embodiments, a glycoengineered polypeptide according to the present disclosure is delivered in an amount effective to reduce levels of anti-PLA2R autoantibody or a fragment or an immune complex comprising the same.

In some embodiments, a glycoengineered polypeptide according to the present disclosure is delivered in an amount effective to reduce levels of anti-THSD7A autoantibody or a fragment or an immune complex comprising the same.

In some embodiments, a glycoengineered polypeptide according to the present disclosure is delivered in an amount effective to reduce levels of anti-NELL1 autoantibody or a fragment or an immune complex comprising the same.

In some embodiments, a glycoengineered polypeptide according to the present disclosure is delivered in an amount effective to reduce levels of anti-EXT1 autoantibody or a fragment or an immune complex comprising the same.

In some embodiments, a glycoengineered polypeptide according to the present disclosure is delivered in an amount effective to reduce levels of anti-EXT2 autoantibody or a fragment or an immune complex comprising the same.

### Combination Therapies

According to the present disclosure, glycoengineered polypeptides may be administered in combination with one or more therapies such as standard of care that is typically used for the treatment and/or management of membranous nephropathy. As will be appreciated with one with skill in the art, the KDIGO 2021 Clinical Practice Guidelines for the Management of Glomerular Diseases, Volume 100, Issue 45, published in October 2021 provides treatment options for patients with membranous nephropathy.

In some embodiments, glycoengineered polypeptides may be administered in combination with one or more pharmaceutical agents. For example, a glycoengineered polypeptide may be administered in combination with one or more therapeutic agents for membranous nephropathy (e.g., iMN) (such as agents that ameliorate symptoms of membranous nephropathy), and/or in combination with one or more other pharmaceutical agents. In some embodiments, glycoengineered polypeptides may be administered in combination with one or more therapies and/or agents that are prescribed by a clinician for treatment of membranous nephropathy (e.g., iMN).

In some embodiments, a pharmaceutical composition according to the present disclosure is administered in combination with one or more additional therapies. In some embodiments, a pharmaceutical composition according to the present disclosure is administered in combination with a VEGF pathway inhibitor, an anti-BAFF antibody, an anti-CD20 antibody, an anti-CD38 antibody, an anti-CD19 antibody, a Janus kinase inhibitor, a SYK inhibitor, a Factor B complement inhibitor, a Factor D complement inhibitor, a C3 complement inhibitor, or a combination thereof. In some embodiments, a pharmaceutical composition according to the present disclosure is administered in combination with one or more therapies and/or agents that are prescribed by a clinician for treatment of membranous nephropathy (e.g., iMN).

In some embodiments, a pharmaceutical composition according to the present disclosure is administered in combination with a VEGF pathway inhibitor.

In some embodiments, a pharmaceutical composition according to the present disclosure is administered in combination with an anti-BAFF antibody. In some embodiments, an anti-BAFF antibody is belilumab.

In some embodiments, a pharmaceutical composition according to the present disclosure is administered in combination with an anti-CD20 antibody. In some embodiments, an anti-CD20 antibody is Rituximab or Oblinutuzumab.

In some embodiments, a pharmaceutical composition according to the present disclosure is administered in combination with an anti-CD38 antibody. In some embodiments, an anti-CD38 antibody is Felzartamab.

In some embodiments, a pharmaceutical composition according to the present disclosure is administered in combination with an anti-CD19 antibody. In some embodiments, an anti-CD19 antibody is VB119.

In some embodiments, a pharmaceutical composition according to the present disclosure is administered in combination with a Janus kinase inhibitor. In some embodiments, a Janus kinase inhibitor is Filgotinib.

In some embodiments, a pharmaceutical composition according to the present disclosure is administered in combination with a SYK inhibitor.

In some embodiments, a pharmaceutical composition according to the present disclosure is administered in combination with a Factor B complement inhibitor. In some embodiments, a Factor B complement inhibitor is Iptacopan.

In some embodiments, a pharmaceutical composition according to the present disclosure is administered in combination with a Factor D complement inhibitor. In some embodiments, a Factor D complement inhibitor is BCX9930.

In some embodiments, a pharmaceutical composition according to the present disclosure is administered in combination with a C3 complement inhibitor. In some embodiments, a C3 complement inhibitor is Pegcetacolpan.

### Characterization of Glycoengineered Polypeptides

The present disclosure provides, among other things, glycoengineered polypeptides that specifically bind to anti-podocyte autoantibodies (e.g., anti-PLA2R autoantibodies, anti-NELL1 autoantibodies and/or anti-THSD7A autoantibodies) and thereby causing degradation of anti-podocyte autoantibodies. In some embodiments, anti-podocyte autoantibodies degradation comprises internalized into a cell for degradation (e.g., by transporting the anti-podocyte autoantibody to a lysosome). In some embodiments, glycoengineered polypeptides specifically binding anti-podocyte autoantibodies according to the present disclosure are characterized in that they inhibit the biological function of anti-podocyte autoantibodies (e.g., binding to podocyte autoantigens) including their ability to form immune complexes and/or activate complement pathways.

In some embodiments, a glycoengineered polypeptide according to the present disclosure is used to degrade and/or reduce the levels of anti-podocyte autoantibodies. In some embodiments, a glycoengineered polypeptide is used to lower anti-podocyte autoantibody levels, such as lowering elevated plasma anti-podocyte autoantibody levels in a subject with membranous nephropathy (e.g., iMN).

In some embodiments, the present disclosure provides glycoengineered polypeptides characterized in that when administered to a cell, tissue, or subject, the glycoengineered polypeptide which is bound to a target via the first moiety and to an endocytic receptor via a second moiety results in degradation of the target.

In some embodiments, degradation comprises internalization into a cell. In some embodiments, degradation comprises lysosomal degradation. In some embodiments, degradation occurs in a liver cell.

In some embodiments, a target is an anti-podocyte autoantibody or a fragment thereof or an immune complex comprising the same. In some embodiments, the immune complex comprises one or more: IgG antibodies, the target, an antigen recognized by the target, complement components, or combinations thereof. In some embodiments, the immune complex comprising complement components comprises a membrane attack complex. In some embodiments, the one or more complement components comprise: C3, C5b, C6, C7, C8, and/or C9.

In some embodiments, a target is an anti-PLA2R autoantibody or a fragment thereof or an immune complex comprising the same.

In some embodiments, a target is an anti-THSD7A autoantibody or a fragment thereof or an immune complex comprising the same.

In some embodiments, a target is an anti-NELL1 autoantibody or a fragment thereof or an immune complex comprising the same.

In some embodiments, a target is an anti-EXT1 autoantibody or a fragment thereof or an immune complex comprising the same.

In some embodiments, a target is an anti-EXT2 autoantibody or a fragment thereof or an immune complex comprising the same.

In some embodiments, the present disclosure provides glycoengineered polypeptides characterized in that when administered to a cell, tissue, or subject, the glycoengineered polypeptide which is bound to an anti-podocyte autoantibody via the first moiety prevents activation of a complement pathway or component thereof. In some embodiments, a complement pathway comprises a classical pathway, an alternative pathway, or a lectin pathway.

In some embodiments, the present disclosure provides glycoengineered polypeptides characterized in that when administered to a cell, tissue, or subject, the glycoengineered polypeptide which is bound to an anti-podocyte autoantibody via the first moiety prevents proteinuria, decreased albumin levels and/or edema.

In some embodiments, the present disclosure provides glycoengineered polypeptides characterized in that when administered to a cell, tissue, or subject, the glycoengineered polypeptide which is bound to an anti-podocyte autoantibody via the first moiety prevents thickening of glomerular capillary walls.

In some embodiments, the present disclosure provides glycoengineered polypeptides characterized in that when administered to a cell, tissue, or subject, the glycoengineered polypeptide which is bound to an anti-podocyte autoantibody via the first moiety prevents changes in structure and/or function of podocytes.

In some embodiments, the present disclosure provides glycoengineered polypeptides characterized in that when administered to a cell, tissue, or subject, the glycoengineered polypeptide which is bound to an anti-podocyte autoantibody via the first moiety prevents apoptosis in podocytes.

In some embodiments, the present disclosure provides glycoengineered polypeptides characterized in that when administered to a cell, tissue, or subject, the glycoengineered polypeptide which is bound to an anti-podocyte autoantibody via the first moiety prevents an increase in intracellular calcium in human podocytes.

In some embodiments, the present disclosure provides glycoengineered polypeptides characterized in that when administered to a cell, tissue, or subject, the glycoengineered polypeptide which is bound to an anti-podocyte autoantibody via the first moiety prevents a decrease in cellular motility and/or adhesion of podocytes.

### Method of Making Glycoengineered Polypeptides

The present disclosure, among other things, provides methods of making a glycoengineered polypeptide comprising a first moiety comprising one or more peptides that specifically binds to a target (e.g., an anti-podocyte autoantibody or a fragment or a complex thereof) and a second moiety comprising one or more glycans conjugated to the first moiety.

A glycoengineered polypeptide disclosed herein can be made using methods disclosed in U.S. Provisional Patent Application No. 63/410,955 filed on Sep. 28, 2022 and U.S. Provisional Patent Application No. 63/410,936, filed on Sep. 28, 2022, and International Patent Application PCT/EP2023/076767 filed on Sep. 27, 2023, the entire contents of each of which are hereby incorporated by reference.

For example, in U.S. 63/410,955, Section 5.3 discloses Leishmania host cells; Section 5.4 discloses exemplary methods of genetically engineering a Leishmania cell for expressing glycoengineered polypeptides, Section 5.5 discloses exemplary methods of culturing Leishmania host cells; and Section 5.6 discloses exemplary uses of Leishmania host cells as an expression system.

As another example, in International Patent Application PCT/EP2023/076767, Section 7.1 discloses Leishmania host cells including modifications that can be made to a Leishmania host cell for producing glycoengineered polypeptides, Section 7.2 disclose methods of genetically engineering Leishmania host cells for producing glycoengineered polypeptides, and Section 7.3 discloses methods of culturing Leishmania host cells. An exemplary method of making glycoengineered polypeptides using Leishmania host cells is provided in Example 1 herein.

In particular, as disclosed in PCT/EP2023/076767, exemplary Leishmania strains that can be used to make glycoengineered polypeptides disclosed herein include: StCGP3558, StCGP4564, StCGP5359, or StCGP5942.

As would be understood by persons with ordinary skill in the art, such methods and host cells can also be used for making glycoengineered polypeptides disclosed herein.

In some embodiments, the one or more glycans of the second moiety are conjugated to the first moiety at one or more glycosylation sites with in vivo glycosylation, e.g., in a cell. In some embodiments, a cell is a Leishmania host cell. In some embodiments, a cell is a glycoengineered yeast host cell, e.g., glycoengineered Pichia pastoris host cell.

In some embodiments, the one or more glycans of the second moiety are conjugated to the first moiety at one or more glycosylation sites with chemical conjugation, e.g., using Click chemistry.

Also disclosed herein are methods for making a glycoengineered polypeptide. In one embodiment, provided herein is a method of producing a glycoengineered polypeptide in vivo, using a Leishmania host cell described herein. In some embodiments, provided herein is a method for producing a glycoengineered polypeptide, said method comprising (i) culturing a Leishmania host cell under conditions suitable for polypeptide production and (ii) isolating said glycoengineered polypeptide. In a specific embodiment, the Leishmania host cell comprises: (a) a recombinant nucleic acid encoding a glycoengineered polypeptide; and (b) a recombinant nucleic acid encoding one or more recombinant N-acetylgalactosamine (GalNAc) transferases. In certain embodiments, the Leishmania host cell is capable of producing glycoengineered polypeptide comprising a biantennary, GalNAc-terminated N-glycan. In particular, the Leishmania host cells provided herein is capable of producing glycoengineered polypeptide comprising an N-glycan of the following structure:

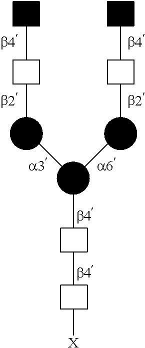

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

In certain embodiments, the glycoengineered polypeptide produced by the Leishmania host cell is a therapeutic polypeptide, i.e., a polypeptide used in the treatment of a disease or disorder. For example, the glycoengineered polypeptide produced by the Leishmania host cell can be peptide or an antibody.

### Leishmania Host Cells

Provided herein are Leishmania host cells for the production of glycoengineered polypeptides disclosed herein, or a population of glycoengineered polypeptides, wherein the Leishmania host cells comprise: (a) a recombinant nucleic acid encoding a glycoengineered polypeptide disclosed herein; and (b) a recombinant nucleic acid encoding one or more recombinant N-acetylgalactosamine (GalNAc) transferases. In certain embodiments, the Leishmania host cells provided herein are capable of producing glycoengineered polypeptides comprising a biantennary, GalNAc-terminated N-glycan. In particular, the Leishmania host cells provided herein are capable of producing glycoengineered polypeptides comprising an N-glycan of the following structure:

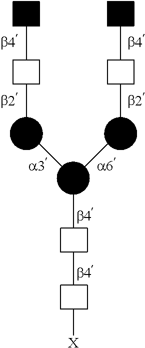

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

In certain embodiments, the Leishmania host cells provided herein comprise a recombinant nucleic acid encoding one or more recombinant N-acetylgalactosamine (GalNAc) transferases disclosed herein. In certain embodiments, the Leishmania host cells provided herein comprise a recombinant nucleic acid encoding one or more additional recombinant glycosyltransferases disclosed herein. In certain embodiments, one or more endogenous enzymes disclosed herein from the glycan biosynthesis pathway of the the Leishmania host cells provided herein have been deleted, mutated and/or functionally inactivated. In certain embodiments, the Leishmania host cells provided herein further comprise a recombinant nucleic acid encoding heterologous UDP-GalNAc biosynthetic pathway proteins capable of generating UDP-GalNAc. In certain embodiments, the Leishmania host cells provided herein comprise a recombinant nucleic acid encoding a heterologous UDP-GalNAc transporter protein capable of transporting UDP-GalNAc to the secretory pathway.

In certain embodiments, the Leishmania host cells provided herein have been genetically engineered such that the formation of an O-linked GlcNAc on a polypeptide produced in the Leishmania host cell is reduced or eliminated. Leishmania host cells that have been genetically engineered to reduce or eliminate the formation of an O-linked GlcNAc are described, for example, in WO 2021/140143, which is incorporated herein by reference in its entirety.x

In certain embodiments, the Leishmania host cells provided herein below are genetically engineered using the methods described herein. In certain embodiments, the Leishmania host cells provided herein below are cultured according to the methods described herein.

Other suitable host cells comprise liver cells, myeloid cells, immune cells, endothelial cells, parenchymal cells or epithelial cells. In some embodiments, the immune cell is a dendritic cell, a macrophage, a monocyte, a microglia cell, a granulocyte or a B lymphocyte.

### Methods of Culturing Leishmania Host Cells

Provided herein are methods for culturing Leishmania host cells. In one embodiment, the Leishmania host cells are cultured using any of the standard culturing techniques known in the art. For example, cells are routinely grown in rich media like Brain Heart Infusion, Trypticase Soy Broth or Yeast Extract, all containing 5 μg/ml Hemin. Additionally, incubation is done at 26° C. in the dark as static or shaking cultures for 2-3 days. In some embodiments, cultures of recombinant cell lines contain the appropriate selective agents. Non-limiting exemplary selective agents are provided in Table 1.

TABLE 1
Selective agents used during transfection (50% concentration for
preselection and 100% concentration for main selection) and standard
culturing of L. tarentolae. Double amounts of the selective agents
could be used if higher selection pressure was intended.

| | | Concentration | | | |
|---|---|---|---|---|---|
| | Resistance | (100%) | | Concentration | |
| Selective | conferring | main selection/ | | (50%) | |
| agent | gene | standard culturing | | preselection | |
| Nourseothricin | sat | 50 | μg/ml | 25 | μg/ml |
| Geneticin | neo | 50 | μg/ml | 25 | μg/ml |
| Paromomycin | neo | 300 | μg/ml | 150 | μg/ml |
| Zeocin | ble | 150 | μg/ml | 75 | μg/ml |
| Hygromycin | hyg | 50 | μg/ml | 25 | μg/ml |
| Blasticidin | bsd | 5 | μg/ml | 2.5 | μg/ml |
| Puromycin | pac | 5 | μg/ml | 2.5 | μg/ml |

In certain embodiments, the Leishmania host cells are cultured in a growth medium comprising GalNAc. In certain embodiments, the growth medium comprises at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, or at least 20 mM GalNAc. In certain embodiments, the growth medium comprises about 1 mM to about 5 mM, about 5 mM to about 10 mM, about 10 mM to about 15 mM, or about 15 mM to about 20 mM GalNAc. In certain embodiments, the growth medium comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM GalNAc. In certain embodiments, the growth medium comprises about about 10 mM GalNAc.

In certain embodiments, the Leishmania host cells are cultured in a growth medium comprising GlcNAc. In certain embodiments, the growth medium comprises at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, or at least 20 mM GlcNAc. In certain embodiments, the growth medium comprises about 1 mM to about 5 mM, about 5 mM to about 10 mM, about 10 mM to about 15 mM, or about 15 mM to about 20 mM GlcNAc. In certain embodiments, the growth medium comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM GlcNAc.

In certain embodiments, a Leishmania host cell may be used as an expression system for making a glycoengineered polypeptide disclosed herein or a population of glycoengineered polypeptides. In certain embodiments, the glycoengineered polypeptide degrader may be a heterologous, non-Leishmania protein, such as a therapeutic protein (e.g., an antibody). Other methods of producing Leishmania host cells for use as expression systems are known and may also be used, for example, see WO 2019/002512, WO 2021/140144 and WO 2021/140143, each of which are incorporated herein by reference in their entirety. Use of Leishmania host cells to make monoclonal antibodies are also known. Exemplary methods are described in WO 2022/053673, which is incorporated herein by reference in its entirety.

The compositions comprising the Leishmania host cells can comprise additional components suitable for maintenance and survival of the Leishmania host cells, and can additionally comprise additional components required or beneficial to the production of glycoengineered bifunctional degraders by the Leishmania host cells, e.g., inducers for inducible promoters, such as arabinose, IPTG.

### Yeast or Filamentous Fungal Host Cells

Provided herein are yeast or filamentous fungal host cells for the production of glycoengineered polypeptides disclosed herein, or a population of glycoengineered polypeptides. In some embodiments, a yeast or filamentous fungal host cell is a K lactis host cells. In some embodiments, a yeast or filamentous fungal host cell is a Pichia pastoris host cell. In some embodiments, a yeast or filamentous fungal host cell is a Pichia methanolica host cell. In some embodiments, a yeast or filamentous fungal host cell is a Hansenula host cell.

Exemplary yeast or filamentous fungal host cells that can be used to produce glycoengineered polypeptides disclosed herein are disclosed in U.S. Pat. No. 8,206,949, the entire contents of which are hereby incorporated by reference.

Exemplary yeast or filamentous fungal host cells that can be used to produce glycoengineered polypeptides disclosed herein are disclosed in U.S. Pat. No. 7,981,660, the entire contents of which are hereby incorporated by reference.

Exemplary yeast or filamentous fungal host cells that can be used to produce glycoengineered polypeptides disclosed herein are disclosed in U.S. Pat. No. 8,883,483, the entire contents of which are hereby incorporated by reference.

In some embodiments, a yeast or filamentous fungal host cell has been genetically modified to produce glycoproteins with a predominant N-glycan glycoform.

In certain embodiments, the yeast or filamentous fungal host cells provided herein are capable of producing glycoengineered polypeptides comprising a biantennary, GalNAc-terminated N-glycan. In particular, the yeast or filamentous fungal host cells provided herein are capable of producing glycoengineered polypeptides comprising an N-glycan of the following structure:

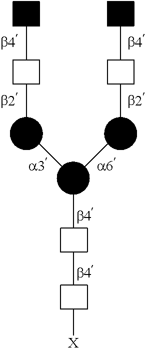

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered polypeptide.

### EXAMPLES

### Example 1: Construction of Glycoengineered Polypeptides Comprising a PLA2R Polypeptide

This example describes the construction of an exemplary glycoengineered polypeptide comprising a portion of a PLA2R polypeptide for use as a potential bait for autoantibody degradation.

The phospholipase A2 receptor, PLA2R (Uniprot Q13018), can exist as a membrane bound form and a soluble form (by shedding). It is a Type I transmembrane glycoprotein with an intracellular C-terminal region. PLA2R has the following domains: a Cysteine rich region “CysR” (or Ricin B-type lectin domain), a Fibronectin-like type II domain (Fn) and a repeat of C-type lectin-like domains “CTLD” called C1 to C8.

Methods: The construction of the glycoengineered polypeptide of PLA2R comprised four major steps. First, the PLA2R protein was engineered to cover the minimal epitopes suitable for binding the diversity of anti-PLA2R autoantibodies from iMN patients, including a fragment comprising the N-terminal Cys rich region,Fn domain, CTLD C1 (also referred to herein as CTLD1) and CTLD C2 (also referred to herein as CTLD2). Second, the native signal peptide was used at the N-terminus of PLA2R to make it suitable for recombinant protein expression and secretion. While a PLA2R native signal peptide was used in this Example, other suitable signal peptides as can be readily envisioned by an ordinarily skilled artisan can also be used. Third, the C terminus of PLA2R was modified with a peptide sequence (“glycotag”) containing a glycosylation consensus site (SEQ ID NO: 37) and a C terminal His tag for purification (SEQ ID NO: 40). Fourth, the native glycosylation sites N93, N315, N433, N454 and N473 were maintained in the polypeptide fragment spanning from the N terminal CysR region, Fn domain and the CTLD C1 and C2 domains (Table 2). These construction steps resulted in a PLA2R glycoengineered polypeptide protein having six N-glycosylation sites (five native sites: N93, N315, N433, N454 and N473, and a sixth engineered glycosylation site within a C terminal glycotag sequence). The sequence of the exemplary PLA2R glycoengineered polypeptide is provided herein as SEQ ID NO: 41.

The corresponding nucleotide sequence was transfected as an expression cassette into a glycoengineered Leishmania tarentolae host cell line. The resulting cell line StCGP6199 (based on parental strain StCGP4564 disclosed herein) was grown in a bioreactor for PLA2R glycoengineered polypeptide expression and secretion. The recombinant protein containing A2GalNAc2 glycans was then purified from the cell supernatant via an IMAC purification and further analyzed (Table 3). Size exclusion chromatography (SEC) was used to quantify aggregation and degradation of the protein.

Results: The glycoengineering process resulted in an exemplary PLA2R glycoengineered polypeptide (SEQ ID NO: 41). An overview of the polypeptide is provided in Table 2. Analytical data of the construct demonstrated that the PLA2R glycoengineered polypeptide has a high percentage of A2GalNAc2 (83.6%) and high purity (94.6% monomer measured by SEC), see also Table 3.

TABLE 2
Overview of the PLA2R glycoengineered
polypeptide and Status Glycosylation

| | Domain | N- | N- | |
|---|---|---|---|---|
| | (aa | glycosylation | glycosylation | A2GalNAc2 |
| Domain | position) | site | site occupancy | % |
| (CysR; Ricin | 38-161 | N93 | >99% | >85% |
| B-type lectin) | | | | |
| (Fibronectin | 173-221 | none | / | / |
| type-I) Fn | | | | |
| C1 | 238-355 | N315 | <10% occupied | >70% |
| C2 | 385-502 | N433 | <1% | |
| C2 | 385-502 | N454 | >90% | >85% |
| | | | (estimated) | (estimated) |
| C2 | 385-502 | N473* | >50% | >80% |
| | | | (estimated) | (estimated) |
| C terminal | | | >99% | >75% |
| glycotag | | | | |

TABLE 3
Analytical data for the PLA2R glycoengineered polypeptide

| | | Aggregation/ | | N-glycosylation |
|---|---|---|---|---|
| | N-glycan | Degradation | Endotoxin | site occupancy |
| Protein | (total) | (SEC) | (LAL) | (Peptide mapping) |
| PLA2R | 83.6% | 94.6% monomer; | <1 EU/mg | 5% 1 site |
| glycoengineered | A2GalNAc2 | 3.9% degradation; | | unoccupied; |
| polypeptide | | 1.5% aggregation | | 45% 2 sites |
| | | | | unoccupied; |
| | | | | 40% 3 sites |
| | | | | unoccupied; |
| | | | | 10% 4 sites |
| | | | | unoccupied |

LOD: limit of detection; EU: Endotoxin units.

### Example 2: Glycoengineered PLA2R Polypeptides Bind to Anti-PLA2R Antibody and Human ASGPR1

This example demonstrates binding of an exemplary glycoengineered polypeptide comprising a PLA2R polypeptide to an anti-PLA2R antibody and to human ASGPR1.

Methods: Surface plasmon resonance (SPR) was used to measure binding kinetics (i.e., ka, kd, KD, equilibrium) of a glycoengineered PLA2R polypeptide. SPR data was collected with a Biacore 8K instrument to characterize interactions between the PLA2R glycoengineered polypeptide and surrogate anti-PLA2R antibody 12-6-5 in multiple assay formats. The surrogate antibody was either captured using the Fc tag or covalently immobilized on the sensor surface, and the PLA2R glycoengineered polypeptide construct was injected in increasing concentrations in a single cycle kinetics experiment. The contact time was 300 s and the dissociation time was 1200 s. The data was fit to a 1:1 binding model with the global fitting software provided by Cytivia. For ASGPR1 binding experiments, biotinylated recombinant human ASGPR1 (Acro Biosystems) was immobilized using streptavidin capture, and increasing concentrations of the PLA2R glycoengineered polypeptide were injected in a single cycle kinetics experiment. ASGPR1 assays included 2 mM CaCl₂) in the running buffer. The data were fit using a heterogeneous binding model using the Cytvia provided software.

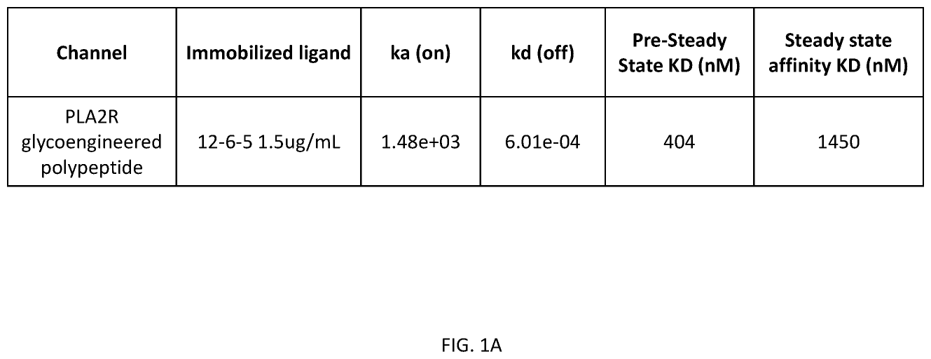

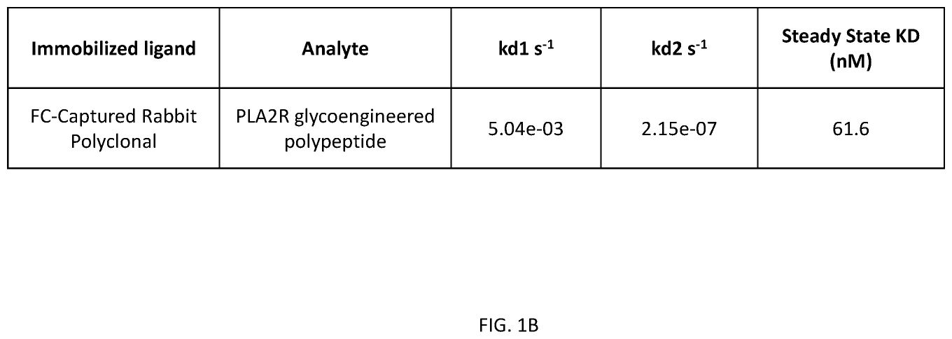

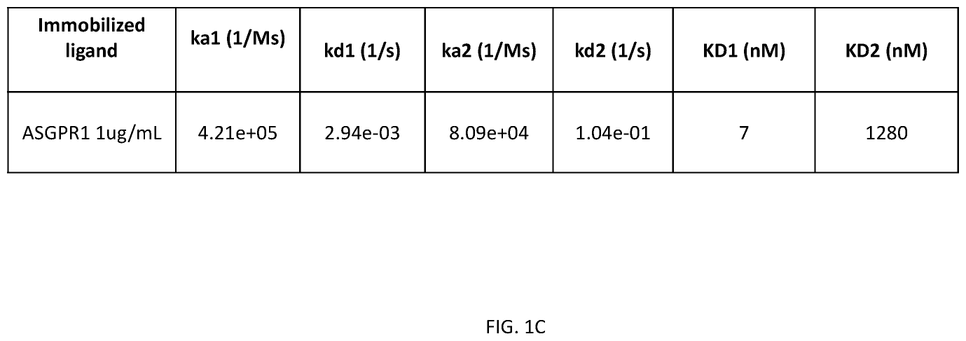

Results: SPR was used to measure the affinity of the PLA2R glycoengineered polypeptide to ASGPR1 and the surrogate anti-PLA2R antibody 12-6-5. When the antibody was covalently immobilized (FIG. 1A), the PLA2R glycoengineered polypeptide displayed a ka1 (i.e., on-rate, association with 12-6-5) of 1.48×10³ 1/Ms, a kd1 (i.e., off-rate, disassociation with 12-6-5) of 6.01×10⁻⁴ l/s, a pre-steady state KD of 404 nM, and a steady state KD of 1450 nM. When the antibody was immobilized using Fc capture (FIG. 1B), the PLA2R glycoengineered polypeptide displayed a kd1 (initial off-rate, disassociation with 12-6-5) of 5.04×10⁻³ l/s, a kd2 of 2.15×10⁻⁷ l/s, and a steady state KD of 61.6 nM. the PLA2R glycoengineered polypeptide also interacted with and showed a high steady state affinity for ASGPR1 (FIG. 1C), with ka2 (i.e., on-rate, association with ASGPR1) of 8.09×10⁴ 1/Ms, a kd2 (i.e., off-rate, disassociation with ASGPR1) of 1.04×10⁻¹ 1/s, and a steady state KD of 1280 nM.

This data shows that an exemplary glycoengineered PLA2R polypeptide can bind to both a target anti-PLA2R antibody and to human ASGPR1 with high affinity. Accordingly, this data supports the development of a glycoengineered polypeptide comprising a PLA2R polypeptide and a glycan moiety for binding to anti-PLA2R autoantibodies and to an endocytic receptor, e.g., ASGPR1, for targeting anti-PLA2R autoantibodies for degradation.

### Example 3: Glycoengineered PLA2R Polypeptides Bind to and Block PLA2R Autoantibodies

This example demonstrates that an exemplary glycoengineered polypeptide comprising a PLA2R polypeptide blocks autoantibodies in iMN patient serum.

Methods: Treatment of iMN patient sera with the PLA2R glycoengineered polypeptide and ELISA to quantify residual anti-PLA2R autoantibodies were carried out as follows. Serum collected from sixteen iMN patients and two control patients with other disorders was diluted 1:100. Diluted serum was left untreated or treated with concentrations of the PLA2R glycoengineered polypeptide ranging from 0 to 20 nM for 30 minutes at room temperature. Anti-PLA2R autoantibodies levels were then evaluated using an anti-PLA2R ELISA kit following the manufacturer instructions (Catalog #EA1254-9601, Euroimmun). Data obtained were quantitatively evaluated with the standard curve using a 4-parameter function. Results are expressed as a relative units per mL (Ru/ML) of PLA2R autoantibodies.

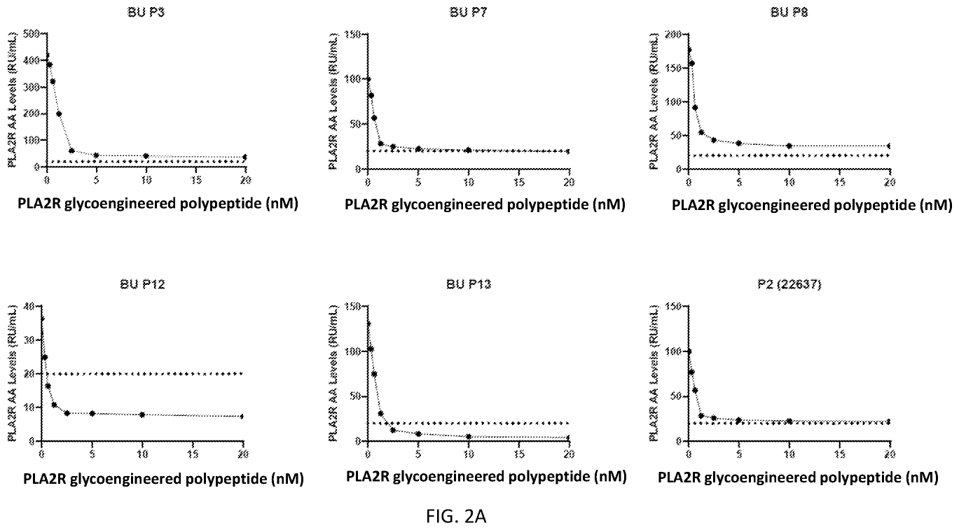

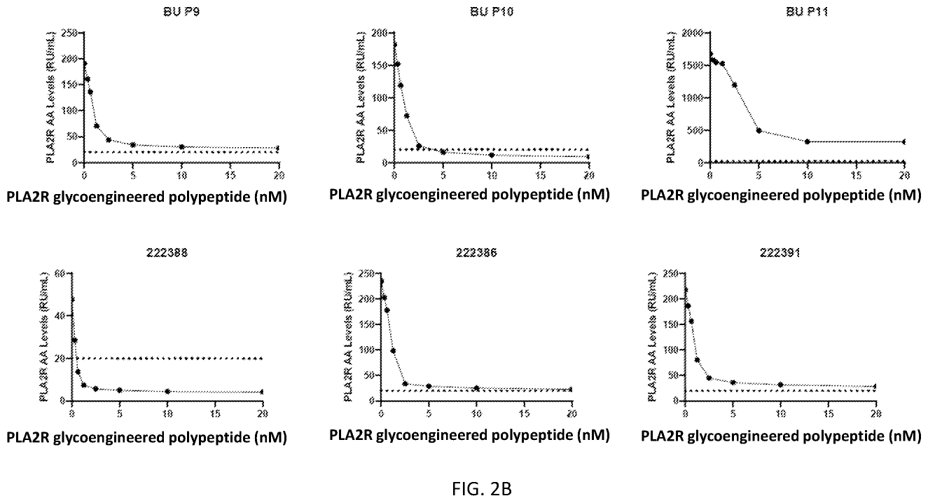

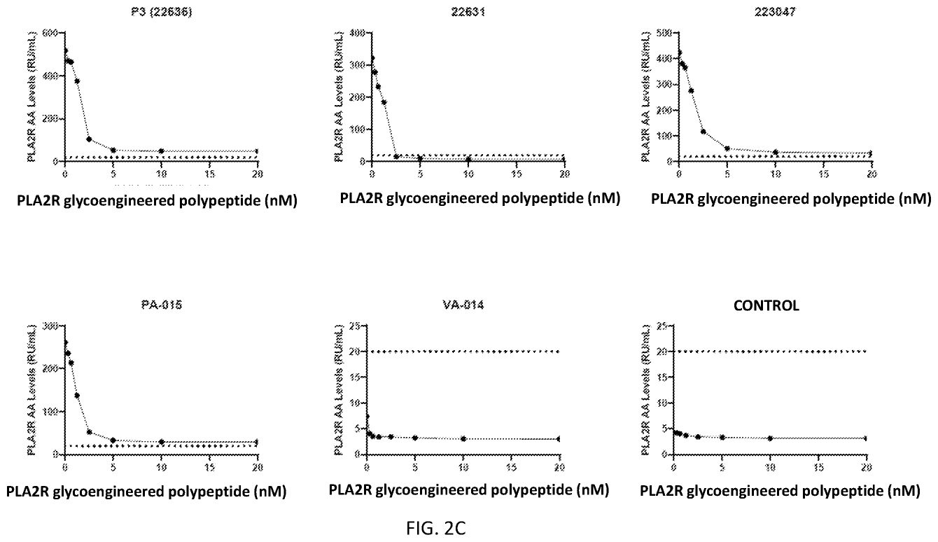

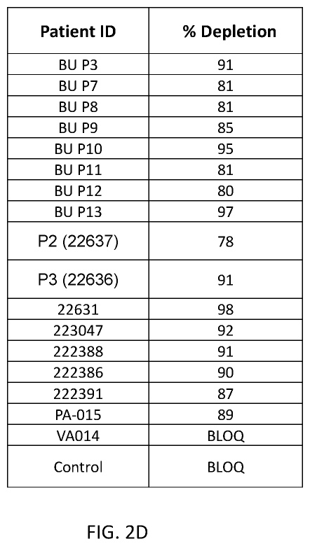

Results: Serum samples from all iMN patients tested showed dose dependent decreases in the level of anti-PLA2R autoantibodies after treatment with the PLA2R glycoengineered polypeptide (FIGS. 2A-2C). Treatment with a concentration of 10 nM of the PLA2R glycoengineered polypeptide was able to block 80% or more of the autoantibodies present in iMN patients serum in 15 out of the 16 iMN patients tested (FIG. 2D). This blocking occurred independently of the initial autoantibody titer (FIGS. 2A-2C), as the depletion of the autoantibody was not correlated with the initial autoantibody titer level, which ranged from about 40 RU/mL to above 1500 RU/mL across patient sera. The two non-iMN patient controls (VA014 and Control) did not show any detectable amount of anti-PLA2R autoantibodies (FIG. 2C, last two panels).

These data suggest that the PLA2R glycoengineered polypeptide can bind and block the activity of autoantibodies in the serum of iMN patients. This data further supports the development of glycoengineered polypeptides comprising a PLA2R polypeptide for use as therapeutic agent in treating and/or preventing iMN (e.g., in a subject having one or more anti-PLA2R autoantibodies). This data also supports the use of glycoengineered polypeptides comprising a PLA2R polypeptide for reducing the level of anti-PLA2R autoantibodies in a subject.

### Example 4: Glycoengineered PLA2R Polypeptides Deplete Anti-PLA2R Autoantibodies

This example demonstrates depletion of anti-PLA2R autoantibodies in iMN patient serum with an exemplary glycoengineered polypeptide comprising a PLA2R polypeptide.

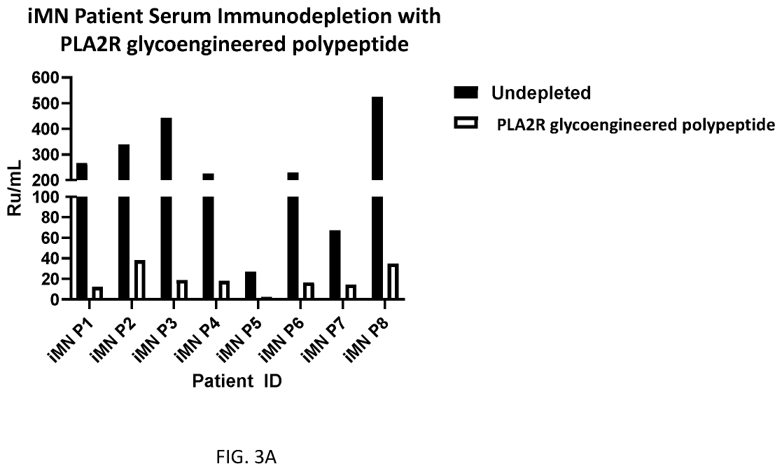

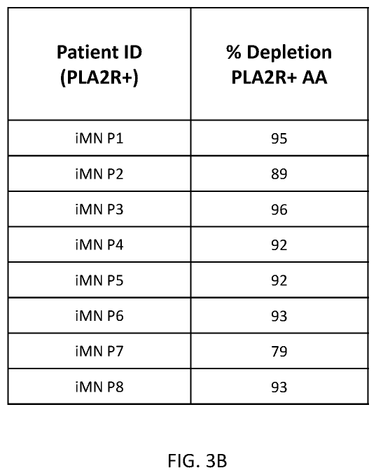

Methods: The PLA2R glycoengineered polypeptide was coupled to Dynabeads (Catalog #10103D, Invitrogen) following the manufacturer's instructions. Sera was diluted 50 times in 500 ul of Binding/Wash Buffer as per manufacturer protocol and 1 mg of the PLA2R glycoengineered polypeptide conjugated beads was added to the samples and incubated for 10 minutes at room temperature. Next, tubes were next placed on a magnet and supernatant collected (depleted sera). Autoantibody levels were evaluated using an anti-PLA2R ELISA kit following the manufacturer instructions (Catalog #EA1254-9601, Euroimmun). Data obtained were quantitatively evaluated with the standard curve using a 4-parameter function. Results are expressed in relative units per mL (RU/mL) in the serum sample before and after depletion with the PLA2R glycoengineered polypeptide coated beads (FIG. 3A) or as a percentage of autoantibody depletion for each patient (FIG. 3B), calculated as 100−(autoantibody level after depletion (white bar for each patient in FIG. 3A) divided by undepleted autoantibody level (black bar for each patient in FIG. 3A)*100).

Results: All sera from iMN patients treated with the PLA2R glycoengineered polypeptide coated beads showed a reduction in the level of PLA2R autoantibodies (FIG. 3A). Treatment with the PLA2R glycoengineered polypeptide coated beads depleted at least 79% of PLA2R autoantibodies from the serum of all iMN patients (FIG. 3B). The percentage of depletion was independent of the starting autoantibodies titers, as the percentage depletion did not correlate with the initial autoantibody titer (shown in the “undepleted” bars in FIG. 3A).

This data suggests that the PLA2R glycoengineered polypeptide can bind to autoantibodies in iMN patient serum in solution. This data supports the development of glycoengineered polypeptides for use as therapeutic agent in treating and/or preventing iMN (e.g., in a subject having one or more anti-PLA2R autoantibodies). This data further supports the use of glycoengineered polypeptides for reducing the level of anti-PLA2R autoantibodies in a subject.

### Example 5: ASGPR-Dependent Internalization and Degradation of Glycoengineered PLA2R Polypeptides in Complex with Anti-PLA2R Antibodies in Hepatocytes

This example demonstrates that complexes comprising an exemplary glycoengineered polypeptide and anti-PLA2R antibody can be internalized and degraded in an ASGPR-dependent manner.

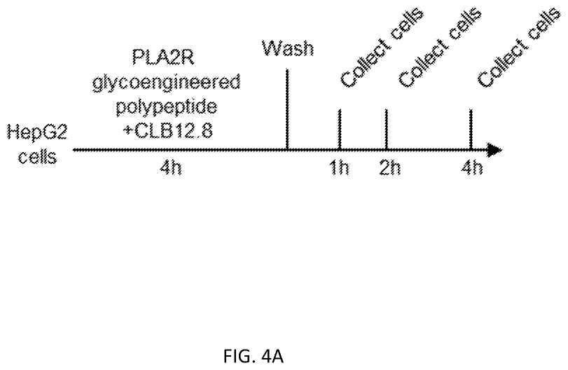

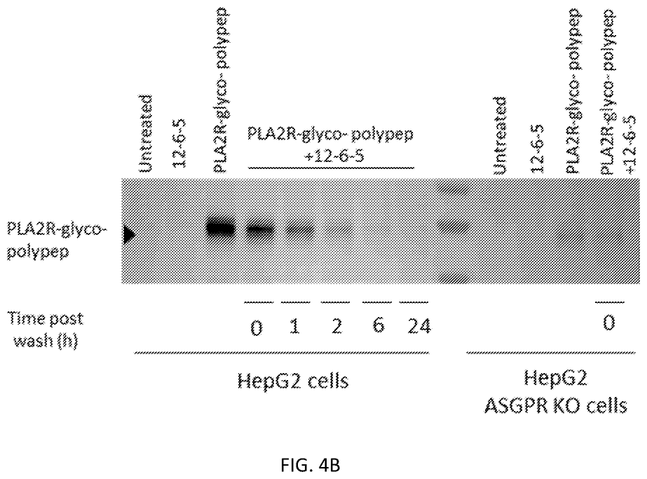

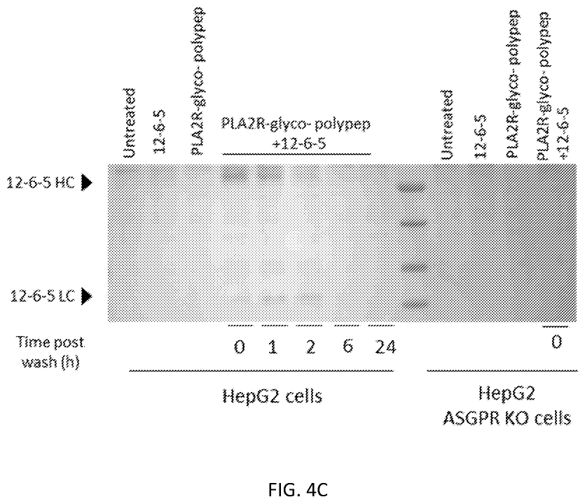

Methods: The experimental set-up is shown in FIG. 4A. The PLA2R glycoengineered polypeptide was pre-incubated with an anti-PLA2R antibody (clone 12-6-5) and allowed to form complexes. The complexes were added to HepG2 cell (wild-type or ASGPR knock-out) for 4 hours for internalization. HepG2 cells were then washed and collected at 0, 1, 2, 6 and 24 hours post wash and analyzed for the PLA2R glycoengineered polypeptide and 12-6-5 internalization by western blot. HepG2 ASGPR knock-out cells were washed and collected at 0 hours post wash only before further analysis. For internalization analysis, cell extracts were analyzed by immunoblotting using anti-His or anti-mouse IgG antibody.

Results: FIG. 4B shows that the PLA2R glycoengineered polypeptide is internalized in HepG2 cells. No change in internalization was observed when the PLA2R glycoengineered polypeptide was added in complex to the surrogate autoantibody anti-PLA2R clone 12-6-5. After the complexes were washed out from the media, the internalized the PLA2R glycoengineered polypeptide was quickly degraded and almost no the PLA2R glycoengineered polypeptide was detected in HepG2 cells 6 hours post-wash (FIG. 4B, left side of blot labelled “HepG2 cells”).

Furthermore, the surrogate autoantibody anti-PLA2R clone 12-6-5 was only internalized when in complexed with the PLA2R glycoengineered polypeptide. As shown in FIG. 4C, the presence of the anti-PLA2R antibody clone 12-5-6 was only detected by Western blot (i.e., internalized) when anti-PLA2R antibody clone 12-5-6 was allowed to form complexes with the PLA2R glycoengineered polypeptide. After the complexes were washed out from the media, anti-PLA2R clone 12-6-5 was also quickly degraded and almost no remaining antibody was detected 6 hours post wash.

No internalization of the PLA2R glycoengineered polypeptide or complexes comprising the PLA2R glycoengineered polypeptide and the anti-PLA2R antibody clone 12-5-6 was observed in HepG2 cells with ASGPR knockout (right side of blots in FIGS. 4B-C, labeled “HepG2 ASGPR KO cells”), confirming that internalization of the complexes is ASGPR dependent.

This data shows that the PLA2R glycoengineered polypeptide and 12-6-5 complexes were internalized and subsequently degraded in hepatocytes, and that this process was dependent on ASGPR. Taken together, these data suggest that the PLA2R glycoengineered polypeptide can promote internalization and degradation of anti-PLA2R autoantibodies in hepatocytes via ASGPR. This data further supports the development of glycoengineered polypeptides for use as a therapeutic agent in treating and/or preventing iMN (e.g., in a subject having one or more anti-PLA2R antibodies). This data also supports the development of glycoengineered polypeptides for reducing the level of anti-PLA2R autoantibodies in a subject.

### ENUMERATED EMBODIMENTS

Embodiment 1. A glycoengineered polypeptide comprising:
(a) a first moiety comprising one or more peptides that specifically binds to an anti-podocyte autoantibody or a fragment or a complex thereof; and
(b) a second moiety comprising one or more glycans conjugated to the first moiety at one or more glycosylation sites.
Embodiment 2. The glycoengineered polypeptide of embodiment 1, wherein the anti-podocyte autoantibody is an anti-PLA2R autoantibody or a fragment or a complex thereof.
Embodiment 3. The glycoengineered polypeptide of embodiment 2, wherein the anti-PLA2R autoantibody binds to a PLA2R polypeptide, or a variant or fragment thereof.
Embodiment 4. The glycoengineered polypeptide of embodiment 1, wherein the anti-podocyte autoantibody is an anti-THSD7A autoantibody or a fragment or a complex thereof.
Embodiment 5. The glycoengineered polypeptide of embodiment 4, wherein the anti-THSD7A autoantibody binds to a THSD7A polypeptide, or a variant or fragment thereof.
Embodiment 6. The glycoengineered polypeptide of embodiment 1, wherein the anti-podocyte autoantibody is an anti-NELL1 autoantibody or a fragment or a complex thereof.
Embodiment 7. The glycoengineered polypeptide of embodiment 6, wherein the anti-NELL1 autoantibody binds to a NELL1 polypeptide, or a variant or fragment thereof.
Embodiment 8. The glycoengineered polypeptide of embodiment 1, wherein the anti-podocyte autoantibody is an anti-NEP autoantibody or a fragment or a complex thereof.
Embodiment 9. The glycoengineered polypeptide of embodiment 8, wherein the anti-NEP autoantibody binds to a NEP polypeptide, or a variant or fragment thereof.
Embodiment 10. The glycoengineered polypeptide of embodiment 1, wherein the anti-podocyte autoantibody is an anti-EXT1 autoantibody or a fragment or a complex thereof.
Embodiment 11. The glycoengineered polypeptide of embodiment 10, wherein the anti-EXT1 autoantibody binds to an EXT1 polypeptide, or a variant or fragment thereof.
Embodiment 12. The glycoengineered polypeptide of embodiment 1, wherein the anti-podocyte autoantibody is an anti-EXT2 autoantibody or a fragment or a complex thereof.
Embodiment 13. The glycoengineered polypeptide of embodiment 12, wherein the anti-EXT2 autoantibody binds to an EXT2 polypeptide, or a variant or fragment thereof.
Embodiment 14. The glycoengineered polypeptide of embodiment 1, wherein the glycoengineered polypeptide is capable of binding to any one, or all, or a combination of: an anti-PLA2R autoantibody or a fragment or a complex thereof;
an anti-THSD7A autoantibody or a fragment or a complex thereof;
an anti-NELL autoantibody or a fragment or a complex thereof;
an anti-NEP autoantibody or a fragment or a complex thereof;
an anti-EXT1 autoantibody or a fragment or a complex thereof; and
an anti-EXT2 autoantibody or a fragment or a complex thereof.
Embodiment 15. The glycoengineered polypeptide of any one of the preceding embodiments, wherein the second moiety specifically binds to one or more endocytic receptors.
Embodiment 16. The glycoengineered polypeptide of embodiment 15, wherein the endocytic receptor is or comprises an endocytic lectin.
Embodiment 17. The glycoengineered polypeptide of embodiment 16, wherein the endocytic receptor is chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.
Embodiment 18. The glycoengineered polypeptide of any one of the preceding embodiments, wherein the glycan comprises a terminal GlcNac.
Embodiment 19. The glycoengineered polypeptide of any one of embodiments 1-17, wherein the glycan comprises a terminal GalNac.
Embodiment 20. The glycoengineered polypeptide of any one of embodiments 1-17, wherein the glycan comprises a terminal Gal.
Embodiment 21. The glycoengineered polypeptide of any one of the preceding embodiments, wherein the glycan is an N-glycan.
Embodiment 22. The glycoengineered polypeptide of embodiment 21, wherein the N-glycan is linked to the glycoengineered polypeptide at 1, 2, 3, 4 or 5 N-glycosylation sites.
Embodiment 23. The glycoengineered polypeptide of any one of the preceding embodiments, wherein the glycan structure comprises GlcNAc2-Man3-GlcNAc2, GalNAc2-GlcNAc2-Man3-GlcNAc2, Gal2-GlcNAc2-Man3-GlcNAc2, GlcNAc1-Man3-GlcNAc2, Gal2-GlcNAc2-Man3-GlcNAc2, Gal1-GlcNAc2-Man3-GlcNAc2, GalNAc1-GlcNAc2-Man3-GlcNAc2, GlcNAc3-Man3-GlcNAc2, GlcNAc4-Man3-GlcNAc2, Gal3-GlcNAc3-Man3-GlcNAc2, GalNAc3-GlcNAc3-Man3-GlcNAc2, GalNAc4-GlcNAc4-Man3-GlcNAc2, Gal4-GlcNAc4-Man3-GlcNAc2, or Man-6-P-N-glycan.
Embodiment 24. The glycoengineered polypeptide of any one of the preceding embodiments, wherein increasing a number of glycan structures on the glycoengineered polypeptide increases the rate of lysosomal degradation as compared to an otherwise similar glycoengineered polypeptide with fewer glycan structures.
Embodiment 25. The glycoengineered polypeptide of any one of the preceding embodiments, wherein a number of glycan structures comprise 1 or more, 2 or more, 3 or more, 4 or more, 5 or more, 6 or more, 7 or more, 8 or more, 9 or more or 10 or more.
Embodiment 26. The glycoengineered polypeptide of any one of the preceding embodiments, wherein the glycan structure comprises a monoantennary structure, biantennary structure, a triantennary structure, or a tetraantennary structure.
Embodiment 27. The glycoengineered polypeptide of any one of the preceding embodiments, wherein the glycan structure comprises a biantennary structure.
Embodiment 28. The glycoengineered polypeptide of embodiment 27, wherein the glycan structure comprises a biantennary GalNAc.
Embodiment 29. The glycoengineered polypeptide of embodiment 28, wherein the biantennary GalNac binds to an asialoglycoprotein receptor (ASGPR) or a fragment or variant thereof, or a complex comprising ASGPR.
Embodiment 30. The glycoengineered polypeptide of any one 21-29, wherein the N-glycan has a structure of:

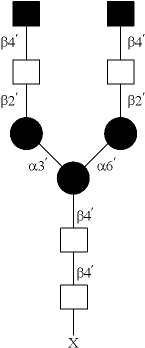

wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.
Embodiment 31. The glycoengineered polypeptide of any one of embodiments 21-30, wherein the N-glycan is conjugated to the glycoengineered polypeptide at at least one, two, three, or four N-glycosylation sites.
Embodiment 32. The glycoengineered polypeptide of any one of embodiments 21-30, wherein the N-glycan is conjugated to the glycoengineered polypeptide at one, two, three, or four N-glycosylation sites.
Embodiment 33. The glycoengineered polypeptide of any one of the preceding embodiments, wherein the N-glycosylation site comprises a consensus sequence of N-X-S/T or N-X-C, wherein X is any amino acid except proline.
Embodiment 34. The glycoengineered polypeptide of any one of the preceding embodiments, wherein the N-glycosylation site is naturally occurring.
Embodiment 35. The glycoengineered polypeptide any one of embodiments 1-33, wherein the N-glycosylation site is engineered into the amino acid sequence of the first moiety, optionally wherein the engineered glycosylation site comprises the sequence of SEQ ID NO: 37 (GGGGANSTAPAPAPA).
Embodiment 36. The glycoengineered polypeptide of any one of embodiments 15-35, wherein the endocytic receptor is or comprises ASGPR or a fragment or variant thereof, or a complex comprising ASGPR.
Embodiment 37. The glycoengineered polypeptide of embodiment 36, wherein when the endocytic receptor is ASGPR, the glycan structure of the second moiety comprises a terminal GalNac.
Embodiment 38. The glycoengineered polypeptide of any one of embodiments 1-2, or 14-37, wherein the first moiety comprises one or more peptides that specifically bind to an anti-PLA2R autoantibody or a fragment thereof.
Embodiment 39. The glycoengineered polypeptide of embodiment 38, wherein the first moiety comprises 1, 2, 3, 4, 5, or more peptides that specifically bind to an anti-PLA2R autoantibody.
Embodiment 40. The glycoengineered polypeptide of embodiment 38 or 39, wherein the one or more peptides that specifically bind to an anti-PLA2R autoantibody are the same.
Embodiment 41. The glycoengineered polypeptide of embodiment 40, wherein the one or more peptides that specifically bind to an anti-PLA2R autoantibody are separated by an intervening sequence.
Embodiment 42. The glycoengineered polypeptide of embodiment 41, wherein the intervening sequence is an IRES, a protease cleavage site, a linker or a spacer or a combination thereof.
Embodiment 43. The glycoengineered polypeptide of embodiment 38 or 39, wherein the one or more peptides that specifically bind to an anti-PLA2R autoantibody are different.
Embodiment 44. The glycoengineered polypeptide of embodiment 43, wherein the one or more peptides that specifically bind to an anti-PLA2R autoantibody are separated by an intervening sequence.
Embodiment 45. The glycoengineered polypeptide of embodiment 44, wherein the intervening sequence is an IRES, a protease cleavage site, a linker or a spacer or a combination thereof.
Embodiment 46. The glycoengineered polypeptide of any one of embodiments 43-45, wherein the different peptides form a spatial epitope.
Embodiment 47. The glycoengineered polypeptide of any one of embodiments 38-46, wherein the one or more peptides that specifically bind to an anti-PLA2R autoantibody are each conjugated to a second moiety.
Embodiment 48. The glycoengineered polypeptide of any one of embodiments 38-46, wherein the one or more peptides that specifically bind to an anti-PLA2R autoantibody are not each conjugated to the second moiety.
Embodiment 49. The glycoengineered polypeptide of any one of embodiments 38-48, wherein the one or more peptides that specifically bind to an anti-PLA2R autoantibody are conjugated to each other.
Embodiment 50. The glycoengineered polypeptide of embodiment 49, wherein the one or more peptides are situated on one polypeptide.
Embodiment 51. The glycoengineered polypeptide of embodiment 49 or 50, wherein the one or more peptides are separated by an intervening amino acid sequence.
Embodiment 52. The glycoengineered polypeptide of embodiment 51, wherein the intervening amino acid sequence is an IRES, a protease cleavage site, a linker or a spacer or a combination thereof.
Embodiment 53. The glycoengineered polypeptide of any one of embodiments 38-52, wherein the glycoengineered polypeptide comprises a first moiety comprising one peptides that specifically binds to an anti-PLA2R autoantibody or a fragment thereof.
Embodiment 54. The glycoengineered polypeptide of any one of embodiments 38-53, wherein the one or more peptides that specifically bind to an anti-PLA2R autoantibody is a soluble peptide.
Embodiment 55. The glycoengineered polypeptide of any one of embodiments 38-54, wherein the one or more peptides that specifically bind to an anti-PLA2R autoantibody comprises an epitope that is recognized by an anti-PLA2R autoantibody.
Embodiment 56. The glycoengineered polypeptide of any one of embodiments 38-55, wherein the one or more peptides that specifically bind to an anti-PLA2R autoantibody comprises a PLA2R polypeptide, or a fragment or a variant thereof.
Embodiment 57. The glycoengineered polypeptide of embodiment 56, wherein the PLA2R polypeptide is provided as SEQ ID NO: 1 with or without a signal peptide, SEQ ID NO: 38 with or without a signal peptide, or SEQ ID NO: 39.
Embodiment 58. The glycoengineered polypeptide of embodiment 56 or 57, wherein the one or more peptides that specifically bind to an anti-PLA2R autoantibody comprises a fragment of a PLA2R polypeptide.
Embodiment 59. The glycoengineered polypeptide of embodiment 58, wherein the fragment comprises at least 5% of a full length PLA2R polypeptide with or without a signal peptide.
Embodiment 60. The glycoengineered polypeptide of embodiment 58, wherein the fragment comprises no more than 99% of a full length PLA2R polypeptide with or without a signal peptide.
Embodiment 61. The glycoengineered polypeptide of any one of embodiments 58-60, wherein the fragment comprises one or more domains of a PLA2R polypeptide.
Embodiment 62. The glycoengineered polypeptide of embodiment 61, wherein a PLA2R polypeptide comprises the following extracellular domains:
an N-terminal cysteine rich (CysR) domain,
a fibronectin 2 domain, and
eight C-type lectin-like domains (CTLD), e.g., a CTLD1 domain, a CTLD2 domain, a CTLD3 domain, a CTLD4 domain, a CTLD5 domain, a CTLD6 domain, a CTLD7 domain, a CTLD8 domain, or combinations thereof.
Embodiment 63. The glycoengineered polypeptide of embodiment 62, wherein the CysR domain has the amino acid sequence of SEQ ID NO: 2.
Embodiment 64. The glycoengineered polypeptide of any one of embodiments 58-63, wherein the fragment comprises SEQ ID NO: 2 or a portion thereof, or a sequence with at least 90% identity to SEQ ID NO 2.
Embodiment 65. The glycoengineered polypeptide of any one of embodiments 58-63, wherein the fragment comprises SEQ ID NO: 3 (WQDKGIFVIQSESLKKCIQAGKSVLTLENCK) or a portion thereof, or a sequence with at least 90% identity to SEQ ID NO: 3.
Embodiment 66. The glycoengineered polypeptide of any one of embodiments 58-63, wherein the fragment comprises SEQ ID NO: 3 without the N-terminal WQD sequence.
Embodiment 67. The glycoengineered polypeptide of embodiment 62, wherein the CTLD1 domain has the amino acid sequence of SEQ ID NO: 4

(NSHICYQFNLLSSLSWSEAHSSCQMQGGTLLSITDETEENFIREHMSSKTVEVWMGLNQ
LDEHAGWQWSDGTPLNYLNWSPEVNFEPFVEDHCGTFSSFMPSAWRSRDCESTLPYICK).

Embodiment 68. The glycoengineered polypeptide of embodiment 67, wherein the fragment comprises SEQ ID NO: 4 or a portion thereof, or a sequence with at least 90% identity to SEQ ID NO 4.
Embodiment 69. The glycoengineered polypeptide of embodiment 62, wherein the CTLD7 domain has the amino acid sequence of SEQ ID NO: 5

(YGNRTYKIINANMTWYAAIKTCLMHKAQLVSITDQYHQSFLTVVLNRLGYAHWIGLFT
TDNGLNFDWSDGTKSSFTFWKDEESSLLGDCVFADSNGRWHSTACESFLQGAIC).

Embodiment 70. The glycoengineered polypeptide of embodiment 69, wherein the fragment comprises SEQ ID NO: 5 or a portion thereof, or a sequence with at least 90% identity to SEQ ID NO 5.
Embodiment 71. The glycoengineered polypeptide of embodiment 62, wherein the CTLD8 domain has the amino acid sequence of SEQ ID NO: 6

(FKSNCYSFSTVLDSMSFEAAHEFCKKEGSNLLTIKDEAENAFLLEELFAFGSSVQMVWL
NAQFDGNNETIKWFDGTPTDQSNWGIRKPDTDYFKPHHCVALRIPEGLWQLSPCQEKKG
FICK).

Embodiment 72. The glycoengineered polypeptide of embodiment 71, wherein the fragment comprises SEQ ID NO: 6 or a portion thereof, or a sequence with at least 90% identity to SEQ ID NO 6.
Embodiment 73. The glycoengineered polypeptide of any one of embodiments 58-72, wherein the fragment comprises one or more additional amino acid residues on the 5′ and/or 3′ end of the sequence.
Embodiment 74. The glycoengineered polypeptide of any one of embodiment 58-73, wherein the fragment comprises a CysR domain, an Fn domain, and one or more CTLD domains (e.g., a CTLD1 domain, a CTLD2 domain, a CTLD3 domain, a CTLD4 domain, a CTLD5 domain, a CTLD6 domain, a CTLD7 domain, a CTLD8 domain).
Embodiment 75. The glycoengineered polypeptide of embodiment 74, wherein the fragment comprises:
(i) a CysR domain;
(ii) a Fn domain;
(iii) a CTLD1 domain; and
(iv) a CTLD2 domain.
Embodiment 76. The glycoengineered polypeptide of any one of embodiments 38-75, wherein the fragment comprises an epitope that is not present or not accessible in a PLA2R polypeptide from a healthy individual or an individual not at risk of developing idiopathic membranous nephropathy.
Embodiment 77. The glycoengineered polypeptide of any one of embodiments 38-76, wherein the one or more peptides that specifically bind to an anti-PLA2R autoantibody comprises a contiguous chain of amino acids comprising at least 5% of the amino acid of SEQ ID NO: 1, SEQ ID NO: 38 or SEQ ID NO: 39.
Embodiment 77.1. The glycoengineered polypeptide of any one of embodiments 38-76, wherein the one or more peptides that specifically bind to an anti-PLA2R autoantibody comprises a sequence having at least 85% identity to:
(i) SEQ ID NO: 1, or
(ii) SEQ ID NO:1 without the signal peptide, optionally wherein the sequence further comprises a different signal peptide, e.g., as disclosed herein.
Embodiment 77.2. The glycoengineered polypeptide of any one of embodiments 38-76, wherein the one or more peptides that specifically bind to an anti-PLA2R autoantibody comprises a sequence having at least 85% identity to:
(i) SEQ ID NO: 38; or
(ii) SEQ ID NO: 38, without the signal peptide, optionally wherein the sequence further comprises a different signal peptide, e.g., as disclosed herein.
Embodiment 77.3. The glycoengineered polypeptide of any one of embodiments 38-76, wherein the one or more peptides that specifically bind to an anti-PLA2R autoantibody comprises a sequence having SEQ ID NO: 39, optionally wherein the sequence further comprises a signal peptide, e.g., as disclosed herein.
Embodiment 77.4. The glycoengineered polypeptide of any one of embodiments 38-76, wherein the one or more peptides that specifically bind to an anti-PLA2R autoantibody comprises a sequence having at least 85% identity to SEQ ID NO: 41.
Embodiment 78. The glycoengineered polypeptide of any one of embodiments 38-77.2, wherein the one or more peptides that specifically bind to an anti-PLA2R autoantibody comprises a full length PLA2R polypeptide.
Embodiment 79. The glycoengineered polypeptide of any one of embodiments 38-78, wherein binding of the anti-PLA2R autoantibody to a PLA2R polypeptide or a variant or fragment thereof:
(i) alters a level and/or activity of PLA2R;
(ii) induces formation and/or accumulation of an immune complex comprising the anti-PLA2R autoantibody and/or the PLA2R polypeptide; and/or
(iii) activates a complement pathway or component thereof.
Embodiment 80. The glycoengineered polypeptide of embodiment 79, wherein the immune complex comprises one or more: IgG antibodies, the anti-PLA2R autoantibody, the PLA2R polypeptide, complement components, or a combination thereof.
Embodiment 81. The glycoengineered polypeptide of embodiment 80, wherein the immune complex comprising one or more complement components comprises a membrane attack complex.
Embodiment 82. The glycoengineered polypeptide of embodiment 80 or 81, wherein the one or more complement components comprise: C3, C5b, C6, C7, C8, and/or C9.
Embodiment 83. The glycoengineered polypeptide of any one of embodiments 38-82, wherein the one or more peptides that specifically bind to an anti-PLA2R autoantibody comprises an antibody agent.
Embodiment 84. The glycoengineered polypeptide of embodiment 83, wherein the antibody agent comprises an antigen binding fragment.
Embodiment 85. The glycoengineered polypeptide of embodiment 84 wherein the antibody agent comprises a full antibody, a Fab fragment, an scFv, a nanobody, a duobody, or a single domain antibody (e.g., a VHH).
Embodiment 86. The glycoengineered polypeptide of any one of embodiments 1, 7, or 14-37 wherein the first moiety comprises one or more peptides that specifically bind to an anti-NELL autoantibody or a fragment thereof.
Embodiment 87. The glycoengineered polypeptide of embodiment 86, wherein the first moiety comprises 1, 2, 3, 4, 5, or more polypeptides that specifically bind to an anti-NELL autoantibody.
Embodiment 88. The glycoengineered polypeptide of embodiment 86 or 87, wherein the one or more peptides that specifically bind to an anti-NELL autoantibody are the same.
Embodiment 89. The glycoengineered polypeptide of embodiment 88, wherein the one or more peptides that specifically bind to an anti-NELL autoantibody are separated by an intervening sequence.
Embodiment 90. The glycoengineered polypeptide of embodiment 88, wherein the intervening sequence is an IRES, a protease cleavage site, a linker or a spacer or a combination thereof.
Embodiment 91. The glycoengineered polypeptide of embodiment 86 or 87, wherein the one or more peptides that specifically bind to an anti-NELL autoantibody are different.
Embodiment 92. The glycoengineered polypeptide of embodiment 91, wherein the one or more peptides that specifically bind to an anti-NELL1 autoantibody are separated by an intervening sequence.
Embodiment 93. The glycoengineered polypeptide of embodiment 92, wherein the intervening sequence is an IRES, a protease cleavage site, a linker or a spacer or a combination thereof.
Embodiment 94. The glycoengineered polypeptide of any one of embodiments 91-93, wherein the different peptides form a spatial epitope.
Embodiment 95. The glycoengineered polypeptide of any one of embodiments 86-94, wherein the one or more peptides that specifically bind to an anti-NELL autoantibody are each conjugated to a second moiety.
Embodiment 96. The glycoengineered polypeptide of any one of embodiments 86-94, wherein the one or more peptides that specifically bind to an anti-NELL autoantibody are not each conjugated to the second moiety.
Embodiment 97. The glycoengineered polypeptide of any one of embodiments 86-96, wherein the one or more peptides that specifically bind to an anti-NELL autoantibody are conjugated to each other.
Embodiment 98. The glycoengineered polypeptide of embodiment 97, wherein the one or more peptides are situated on one polypeptide.
Embodiment 99. The glycoengineered polypeptide of embodiment 97 or 98, wherein the one or more peptides are separated by an intervening amino acid sequence.
Embodiment 100. The glycoengineered polypeptide of embodiment of embodiment 99, wherein the intervening amino acid sequence is an IRES, a protease cleavage site, a linker or a spacer or a combination thereof.
Embodiment 101. The glycoengineered polypeptide of any one of embodiments 86-Embodiment 100. wherein the glycoengineered polypeptide comprises a first moiety comprising one polypeptide that specifically binds to an anti-NELL autoantibody or a fragment thereof.
Embodiment 102. The glycoengineered polypeptide of any one of embodiments 86-101, wherein the one or more peptides that specifically bind to an anti-NELL autoantibody is a soluble polypeptide.
Embodiment 103. The glycoengineered polypeptide of any one of embodiments 86-101, wherein the one or more peptides that specifically bind to an anti-NELL autoantibody comprises an epitope that is recognized by an anti-NELL autoantibody.
Embodiment 104. The glycoengineered polypeptide of any one of embodiments 86-103, wherein the one or more peptides that specifically bind to an anti-NELL autoantibody comprises a NELL polypeptide, or a fragment or a variant thereof.
Embodiment 105. The glycoengineered polypeptide of embodiment 104, wherein the NELL polypeptide is provided as SEQ ID NO: 9.
Embodiment 106. The glycoengineered polypeptide of embodiment 104 or 105, wherein the one or more peptides that specifically bind to an anti-NELL autoantibody comprises a fragment of a NELL polypeptide.
Embodiment 107. The glycoengineered polypeptide of embodiment 106, wherein the fragment comprises at least 5% of a full length NELL polypeptide.
Embodiment 108. The glycoengineered polypeptide of embodiment 106, wherein the fragment comprises no more than 99% of a full length NELL polypeptide.
Embodiment 109. The glycoengineered polypeptide of any one of embodiments 106-108, wherein the fragment comprises one or more domains of a NELL polypeptide.
Embodiment 110. The glycoengineered polypeptide of embodiment 106-109 wherein the fragment comprises an epitope that is not present or not accessible in a NELL polypeptide from a healthy individual or an individual not at risk of developing idiopathic membranous nephropathy.
Embodiment 111. The glycoengineered polypeptide of any one of embodiments 86-110, wherein the one or more peptides that specifically bind to an anti-NELL autoantibody comprises a contiguous chain of amino acids comprising at least 5% of the amino acid of SEQ ID NO: 9.
Embodiment 112. The glycoengineered polypeptide of any one of embodiments 86-111, wherein the one or more peptides that specifically bind to an anti-NELL autoantibody comprises a full length NELL polypeptide.
Embodiment 113. The glycoengineered polypeptide of any one of embodiments 86-112, wherein the one or more peptides that specifically bind to an anti-NELL autoantibody comprises an antibody agent.
Embodiment 114. The glycoengineered polypeptide of embodiment 113, wherein the antibody agent comprises an antigen binding fragment.
Embodiment 115. The glycoengineered polypeptide of embodiment 114, wherein the antibody agent comprises a full antibody, a Fab fragment, an scFv, a nanobody, a duobody or a single domain antibody (e.g., a VHH).
Embodiment 116. The glycoengineered polypeptide of any one of embodiments 1, 3 or 14-37, wherein the first moiety comprises one or more peptides that specifically bind to an anti-THSD7A autoantibody or a fragment thereof.
Embodiment 117. The glycoengineered polypeptide of embodiment 116, wherein the first moiety comprises 1, 2, 3, 4, 5, or more polypeptides that specifically bind to an anti-THSD7A autoantibody.
Embodiment 118. The glycoengineered polypeptide of embodiment 116 or 117, wherein the one or more peptides that specifically bind to an anti-THSD7A autoantibody are the same.
Embodiment 119. The glycoengineered polypeptide of embodiment 116, wherein the one or more peptides that specifically bind to an anti-THSD7A autoantibody are separated by an intervening sequence.
Embodiment 120. The glycoengineered polypeptide of embodiment 119, wherein the intervening sequence is an IRES, a protease cleavage site, a linker or a spacer or a combination thereof.
Embodiment 121. The glycoengineered polypeptide of embodiment 116 or 117, wherein the one or more peptides that specifically bind to an anti-THSD7A autoantibody are different.
Embodiment 122. The glycoengineered polypeptide of embodiment 121, wherein the one or more peptides that specifically bind to an anti-THSD7A autoantibody are separated by an intervening sequence.
Embodiment 123. The glycoengineered polypeptide of embodiment 122, wherein the intervening sequence is an IRES, a protease cleavage site, a linker or a spacer or a combination thereof.
Embodiment 124. The glycoengineered polypeptide of any one of embodiments 121-123, wherein the different peptides form a spatial epitope.
Embodiment 125. The glycoengineered polypeptide of any one of embodiments 116-124, wherein the one or more peptides that specifically bind to an anti-THSD7A autoantibody are each conjugated to a second moiety.
Embodiment 126. The glycoengineered polypeptide of any one of embodiments 116-124, wherein the one or more peptides that specifically bind to an anti-THSD7A autoantibody are not each conjugated to the second moiety.
Embodiment 127. The glycoengineered polypeptide of any one of embodiments 116-126, wherein the one or more peptides that specifically bind to an anti-THSD7A autoantibody are conjugated to each other.
Embodiment 128. The glycoengineered polypeptide of embodiment 127, wherein the one or more peptides are situated on one polypeptide.
Embodiment 129. The glycoengineered polypeptide of embodiment 127 or 128, wherein the one or more peptides are separated by an intervening amino acid sequence.
Embodiment 130. The glycoengineered polypeptide of embodiment of embodiment 129, wherein the intervening amino acid sequence is an IRES, a protease cleavage site, a linker or a spacer or a combination thereof.
Embodiment 131. The glycoengineered polypeptide of any one of embodiments 116-130, wherein the glycoengineered polypeptide comprises a first moiety comprising one polypeptide that specifically binds to an anti-THSD7A autoantibody or a fragment thereof.
Embodiment 132. The glycoengineered polypeptide of any one of embodiments 116-131, wherein the one or more peptides that specifically bind to an anti-THSD7A autoantibody is a soluble polypeptide.
Embodiment 133. The glycoengineered polypeptide of any one of embodiments 116-132, wherein the one or more peptides that specifically bind to an anti-THSD7A autoantibody comprises an epitope that is recognized by an anti-THSD7A autoantibody.
Embodiment 134. The glycoengineered polypeptide of any one of embodiments 116-133, wherein the one or more peptides that specifically bind to an anti-THSD7A autoantibody comprises a THSD7A polypeptide, or a fragment or a variant thereof.
Embodiment 135. The glycoengineered polypeptide of embodiment 135, wherein the THSD7A polypeptide is provided as SEQ ID NO: 10.
Embodiment 136. The glycoengineered polypeptide of embodiment 134 or 135, wherein the one or more peptides that specifically bind to an anti-THSD7A autoantibody comprises a fragment of a THSD7A polypeptide.
Embodiment 137. The glycoengineered polypeptide of embodiment 136, wherein the fragment comprises at least 5% of a full length THSD7A polypeptide.
Embodiment 138. The glycoengineered polypeptide of embodiment 136, wherein the fragment comprises no more than 99% of a full length THSD7A polypeptide.
Embodiment 139. The glycoengineered polypeptide of any one of embodiments 136-138, wherein the fragment comprises one or more domains of a THSD7A polypeptide.
Embodiment 140. The glycoengineered polypeptide of embodiment 139, wherein the THSD7A polypeptide comprises the following extracellular domains: 21 thrombospondin type 1 domains (TSP) and a coiled coil domain.
Embodiment 141. The glycoengineered polypeptide of embodiment 140, wherein the TSP domain comprises a TSP-1 domain.
Embodiment 142. The glycoengineered polypeptide of embodiment 140, wherein the TSP-1 domain has the amino acid sequence of SEQ ID NO: 11

(TLYLWKTGPWGRCMGDECGPGGIQTRAVWCAHVEGWTTLHTNCKQAERPNNQQNCF
KVCD).

Embodiment 143. The glycoengineered polypeptide of any one of embodiments 136-142, wherein the fragment comprises SEQ ID NO: 11 or a portion thereof, or a sequence with at least 90% identity to SEQ ID NO 11.
Embodiment 144. The glycoengineered polypeptide of any one of embodiments 136-142, glycoengineered wherein the fragment comprises SEQ ID NO: 12 or a portion thereof, or a sequence with at least 90% identity to SEQ ID NO 12.
Embodiment 145. The glycoengineered polypeptide of embodiment 140, wherein the TSP domain comprises a TSP-2 Domain.
Embodiment 146. The glycoengineered polypeptide of embodiment 145, wherein the TSP-2 domain has the amino acid sequence of SEQ ID NO: 17.
Embodiment 147. The glycoengineered polypeptide any one of embodiments 136-142 or 145-146, wherein the fragment comprises SEQ ID NO: 17 or a portion thereof, or a sequence with at least 90% identity to SEQ ID NO 17.
Embodiment 148. The glycoengineered polypeptide any one of embodiments 136-147, wherein the fragment comprises one or more additional amino acid residues on the 5′ and/or 3′ end of the sequence.
Embodiment 149. The glycoengineered polypeptide of any one of embodiments 136-148, wherein the fragment comprises one or more TSP domains.
Embodiment 150. The glycoengineered polypeptide of embodiment 149, wherein the fragment comprises a TSP-1 domain and a TSP-2 domain.
Embodiment 151. The glycoengineered polypeptide of embodiment 149 or 150, wherein the fragment comprises SEQ ID NO: 18 or a portion thereof, or a sequence with at least 90% identity to SEQ ID NO 18.
Embodiment 152. The glycoengineered polypeptide of embodiment 150, wherein the fragment comprises one or more additional amino acid residues on the 5′ and/or 3′ end of the sequence.
Embodiment 153. The glycoengineered polypeptide any one of embodiments 136-152, wherein the fragment comprises an epitope that is not present or not accessible in a THSD7A polypeptide from a healthy individual or an individual not at risk of developing idiopathic membranous nephropathy.
Embodiment 154. The glycoengineered polypeptide of any one of embodiments 116-153, wherein the one or more peptides that specifically bind to an anti-THSD7A autoantibody comprises a contiguous chain of amino acids comprising at least 5% of the amino acid of SEQ ID NO: 1.
Embodiment 155. The glycoengineered polypeptide of any one of embodiments 116-154, wherein the one or more peptides that specifically bind to an anti-THSD7A autoantibody comprises a full length THSD7A polypeptide.
Embodiment 156. The glycoengineered polypeptide of any one of embodiments 116-155, wherein the one or more peptides that specifically bind to an anti-THSD7A autoantibody comprises an antibody agent.
Embodiment 157. The glycoengineered polypeptide of embodiment 156, wherein the antibody agent comprises an antigen binding fragment.
Embodiment 158. The glycoengineered polypeptide of embodiment 157, wherein the antibody agent comprises a full antibody, a Fab fragment, an scFv, a nanobody, a duobody, or a single domain antibody (e.g., a VHH).
Embodiment 159. The glycoengineered polypeptide of any the preceding embodiments wherein the first moiety comprises:
(i) one or more anti-PLA2R autoantibody binding polypeptides;
(ii) one or more anti-THSD7A autoantibody binding polypeptides; and/or
(iii) one or more anti-NELL autoantibody binding polypeptides;
Embodiment 160. The glycoengineered polypeptide of embodiment 159, wherein (i)-(iii) or combinations thereof are situated on the same polypeptide.
Embodiment 161. The glycoengineered polypeptide of embodiment 159, wherein (i)-(iii) or combinations thereof are situated on different polypeptides.
Embodiment 162. The glycoengineered polypeptide of any one of embodiments 159-161, wherein each of (i)-(iii) are conjugated to a second moiety.
Embodiment 163. The glycoengineered polypeptide of any one of the preceding embodiments, wherein the polypeptide comprises one or more additional elements, e.g., a linker, a leader peptide, a spacer, a protease cleavage site, an IRES, or a combination thereof.
Embodiment 164. The glycoengineered polypeptide of any one of the preceding embodiments, wherein the second moiety is conjugated to the first moiety at at least one, two, three, or four N-glycosylation sites.
Embodiment 165. The glycoengineered polypeptide of any one of the preceding embodiments, wherein the second moiety is conjugated to the first moiety at one, two, three, or four N-glycosylation sites.
Embodiment 166. The glycoengineered polypeptide of any one of the preceding embodiments, wherein the N-glycosylation site comprises a consensus sequence of N-X-S/T or N-X-C, wherein X is any amino acid except proline.
Embodiment 167. The glycoengineered polypeptide of any one of the preceding embodiments, wherein the second moiety is conjugated to the first moiety in vivo.
Embodiment 168. The glycoengineered polypeptide of embodiment 167, wherein the conjugation occurs in a cell.
Embodiment 169. The glycoengineered polypeptide of embodiment 168, wherein the cell is a leishmania cell.
Embodiment 170. The glycoengineered polypeptide of any one of embodiments 1-166, wherein the second moiety is conjugated to the first moiety by chemical conjugation.
Embodiment 171. The glycoengineered polypeptide of embodiment 170 wherein chemical conjugation comprises click chemistry.
Embodiment 172. The glycoengineered polypeptide of any one of the preceding embodiments, wherein the first moiety is an antibody and the antibody is a monoclonal or polyclonal antibody.
Embodiment 173. The glycoengineered polypeptide of embodiment 172, wherein the antibody is a recombinant antibody.
Embodiment 174. The glycoengineered polypeptide of embodiment 172 or 173, wherein the antibody is humanized, chimeric or fully human.
Embodiment 175. The glycoengineered polypeptide of any one of embodiments 172-174, wherein the antibody has a glycan to protein ratio of 2 to 1, 4 to 1, 6 to 1, 8 to 1, or 10 to 1.
Embodiment 176. The glycoengineered polypeptide of any one of embodiments 172-175, wherein the antibody is glycosylated at a predetermined and specific residue.
Embodiment 177. The glycoengineered polypeptide of any one of the preceding embodiments, characterized in that when administered to a cell, tissue, or subject, the glycoengineered polypeptide which is bound to an anti-podocyte autoantibody via the first moiety and to an endocytic receptor via a second moiety results in degradation of the target.
Embodiment 178. The glycoengineered polypeptide of embodiment 177, wherein the anti-podocyte autoantibody is:
an anti-PLA2R autoantibody or a fragment or a complex thereof;
an anti-THSD7A autoantibody or a fragment or a complex thereof; and/or an anti-NELL autoantibody or a fragment or a complex thereof.
Embodiment 179. The glycoengineered polypeptide of embodiment 177 or 178, wherein degradation comprises internalization into a cell.
Embodiment 180. The glycoengineered polypeptide of embodiment 179, wherein the anti-podocyte autoantibody and the glycoengineered polypeptide are internalized.
Embodiment 181. The glycoengineered polypeptide of embodiment 180, wherein degradation comprises lysosomal degradation.
Embodiment 182. The glycoengineered polypeptide of embodiment 180 or 181, wherein degradation occurs in a liver cell.
Embodiment 183. The glycoengineered polypeptide of any one of the preceding embodiments, characterized in that when administered to a cell, tissue, or subject the glycoengineered polypeptide which is bound to an anti-podocyte autoantibody via the first moiety prevents formation and/or accumulation of an immune complex.
Embodiment 184. The glycoengineered polypeptide of embodiment 183, wherein the immune complex comprises one or more: IgG antibodies, the anti-podocyte autoantibody, an antigen recognized by the anti-podocyte autoantibody, complement components, or combinations thereof.
Embodiment 185. The glycoengineered polypeptide of embodiment 183, wherein the immune complex comprising complement components comprises a membrane attack complex.
Embodiment 186. The glycoengineered polypeptide of embodiment 184 or 185, wherein the one or more complement components comprise: C3, C5b, C6, C7, C8, and/or C9.
Embodiment 187. The glycoengineered polypeptide of any one of the preceding embodiments, characterized in that when administered to a cell, tissue, or subject the glycoengineered polypeptide which is bound to an anti-podocyte autoantibody via the first moiety prevents activation of a complement pathway or component thereof.
Embodiment 188. The glycoengineered polypeptide of embodiment 187, wherein a complement pathway comprises a classical pathway, an alternative pathway, or a lectin pathway.
Embodiment 189. The glycoengineered polypeptide of any one of the preceding embodiments, characterized in that when administered to a cell, tissue, or subject the glycoengineered polypeptide which is bound to an anti-podocyte autoantibody via the first moiety prevents proteinuria, decreased albumin levels and/or edema.
Embodiment 190. The glycoengineered polypeptide of any one of the preceding embodiments, characterized in that when administered to a cell, tissue, or subject the glycoengineered polypeptide which is bound to an anti-podocyte autoantibody via the first moiety prevents thickening of glomerular capillary walls.
Embodiment 191. The glycoengineered polypeptide of any one of the preceding embodiments, characterized in that when administered to a cell, tissue, or subject the glycoengineered polypeptide which is bound to an anti-podocyte autoantibody via the first moiety prevents changes in structure and/or function of podocytes.
Embodiment 192. The glycoengineered polypeptide of embodiment 191, wherein the glycoengineered polypeptide which is bound to an anti-podocyte autoantibody via the first moiety prevents apoptosis in podocytes.
Embodiment 193. The glycoengineered polypeptide of embodiment 191 or 192, wherein the glycoengineered polypeptide which is bound to an anti-podocyte autoantibody via the first moiety prevents an increase in intracellular calcium in human podocytes.
Embodiment 194. The glycoengineered polypeptide of any one of embodiments 191-193, wherein the glycoengineered polypeptide which is bound to an anti-podocyte autoantibody via the first moiety prevents a decrease in cellular motility and/or adhesion of podocytes.
Embodiment 195. The glycoengineered polypeptide of any one of embodiments 183-194, wherein the anti-podocyte autoantibody is:
an anti-PLA2R autoantibody or a fragment or a complex thereof;
an anti-THSD7A autoantibody or a fragment or a complex thereof; and/or an anti-NELL autoantibody or a fragment or a complex thereof.
Embodiment 196. A polynucleotide encoding the glycoengineered polypeptide of any one of the preceding embodiments.
Embodiment 197. The polynucleotide of embodiment 196, wherein the polynucleotide comprises a portion of the nucleotide sequence of SEQ ID NO: 13 or a codon-optimized version thereof.
Embodiment 198. The polynucleotide of embodiment 197, wherein the polynucleotide comprises at least 10% of SEQ ID NO: 13, or a codon-optimized version thereof.
Embodiment 199. The polynucleotide of embodiment 196, wherein the polynucleotide comprises a portion of the nucleotide sequence of SEQ ID NO:17 or a codon-optimized version thereof.
Embodiment 200. The polynucleotide of embodiment 199, wherein the polynucleotide comprises at least 10% of SEQ ID NO: 17, or a codon-optimized version thereof.
Embodiment 201. The polynucleotide of embodiment 196, wherein the polynucleotide comprises a portion of the nucleotide sequence of SEQ ID NO:20 or a codon-optimized version thereof.
Embodiment 202. The polynucleotide of embodiment 201, wherein the polynucleotide comprises at least 10% of SEQ ID NO: 20, or a codon-optimized version thereof.
Embodiment 203. A composition comprising a glycoengineered polypeptide of any one of the preceding embodiments.
Embodiment 204. The composition of embodiment 203, wherein the composition comprises a glycoengineered polypeptide comprising a first moiety that specifically binds to an anti-PLA2R autoantibody or a fragment or a complex thereof.
Embodiment 205. The composition of embodiment 204, wherein the composition comprises a glycoengineered polypeptide comprising a first moiety that specifically binds to an anti-THSD7A autoantibody or a fragment or a complex thereof.
Embodiment 206. The composition of embodiment 204, wherein the composition comprises a glycoengineered polypeptide comprising a first moiety that specifically binds to an anti-NELL autoantibody or a fragment or a complex thereof.
Embodiment 207. The composition of embodiment 204, wherein the composition comprises:
(i) a first glycoengineered polypeptide comprising a first moiety that specifically binds to an anti-PLA2R autoantibody or a fragment or a complex thereof;
(ii) a second glycoengineered polypeptide comprising a first moiety that specifically binds to an anti-THSD7A autoantibody or a fragment or a complex thereof; and/or
(iii) a third glycoengineered polypeptide comprising a first moiety that specifically binds to an anti-NELL autoantibody or a fragment or a complex thereof.
Embodiment 208. A composition comprising a population of glycoengineered polypeptides of any one of embodiments 1-195, wherein the population of glycoengineered polypeptides has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s).
Embodiment 209. The composition of embodiment 208, wherein the homogeneity of the N-glycan profile at one or more of the N-glycosylation sites is determined by N-glycan analysis, glycopeptide analysis or intact protein analysis.
Embodiment 210. The composition of embodiment 209, wherein the N-glycan profile comprises about 30% to 40%, about 40% to about 50%, about 50% to about 60%, about 60% to about 70%, about 70% to about 80%, about 80% to about 90%, or about 90% to about 100% of the N-glycan of the structure provided in embodiment 30.
Embodiment 211. The composition of embodiment 209, has an N-glycan profile comprising about 30% to about 40%, about 40% to about 50%, about 50% to about 60%, about 60% to about 70%, about 70% to about 80%, about 80% to about 90%, or about 90% to about 100% of the N-glycan of the structure provided in embodiment 30 among all glycans in the N-glycan profile.
Embodiment 212. The composition of any one of embodiments 203-211, wherein the composition is a pharmaceutical composition.
Embodiment 213. The composition of embodiment 212, wherein the pharmaceutical composition comprises one or more excipients.
Embodiment 214. A Leishmania host cell expressing a glycoengineered polypeptide of any one of embodiments 1-195.
Embodiment 215. The host cell of embodiment 214, wherein the cell comprises a polynucleotide sequence encoding a glycoengineered polypeptide.
Embodiment 216. A method comprising: administering to a subject a pharmaceutical composition of embodiment 212 or 213.
Embodiment 217. The method of embodiment 216, wherein the subject has or is diagnosed as having idiopathic membranous nephropathy (iMN).
Embodiment 218. The method of embodiment 217, wherein the method is a treatment method.
Embodiment 219. The method of embodiment 217, wherein the method is a prevention method.
Embodiment 220. A method of treating and/or preventing idiopathic membranous nephropathy (iMN) in a subject, the method comprising, administering to a subject a pharmaceutical composition of embodiment 212 or 213.
Embodiment 221. The method of any one of embodiments 216-220, wherein the glycoengineered polypeptide is capable of simultaneously binding to an anti-podocyte autoantibody with the first moiety and binding to an endocytic receptor-expressing cell with the second moiety, thereby causing the anti-podocyte autoantibody to be internalized into a cell.
Embodiment 222. The method of embodiment 221, wherein internalization comprises transporting to a lysosome and/or degradation.
Embodiment 223. The method of embodiment 221 or 222, wherein the anti-podocyte autoantibody and the glycoengineered polypeptide are internalized.
Embodiment 224. The method of any one of embodiments 221-223, wherein the anti-podocyte autoantibody comprises: an anti-PLA2R autoantibody or a fragment or a complex thereof; an anti-THSD7A autoantibody or a fragment or a complex thereof; an anti-NELL autoantibody or a fragment or a complex thereof, or a combination thereof.
Embodiment 225. The method of any one of embodiments 221-224, wherein the endocytic receptor is ASGPR or a variant or fragment thereof.
Embodiment 226. The method of any one of embodiments 221-225, wherein the subject has increased levels of an anti-podocyte autoantibody as compared to a subject who does not have iMN.
Embodiment 227. The method of any one of embodiments 221-226, wherein the anti-podocyte autoantibody comprises: an anti-PLA2R autoantibody or a fragment or a complex thereof; an anti-THSD7A autoantibody or a fragment or a complex thereof; an anti-NELL autoantibody or a fragment or a complex thereof, or a combination thereof.
Embodiment 228. The method of any one of embodiments 221-227, wherein administration of the pharmaceutical composition reduces a level of an anti-podocyte autoantibody as compared to a subject who has not been administered the pharmaceutical composition or as compared to the same subject prior to administration of the pharmaceutical composition.
Embodiment 229. The method of any one of embodiments 221-228, wherein administration of the pharmaceutical composition reduces and/or prevents formation and/or accumulation of an immune complex as compared to a subject who has not been administered the pharmaceutical composition or as compared to the same subject prior to administration of the pharmaceutical composition.
Embodiment 230. The method of embodiment 229, wherein the immune complex comprises one or more: IgG antibodies, the anti-podocyte autoantibody, an antigen recognized by the anti-podocyte autoantibody, complement components, or combinations thereof.
Embodiment 231. The method of embodiment 230, wherein the immune complex comprising one or more complement components comprises a membrane attack complex.
Embodiment 232. The method of embodiment 230 or 231, wherein the one or more complement components comprise: C3, C5b, C6, C7, C8, and/or C9.
Embodiment 233. The method of any one of embodiments 221-232, wherein administration of the pharmaceutical composition reduces activation of a complement pathway or component thereof as compared to a subject who has not been administered the pharmaceutical composition or as compared to the same subject prior to administration of the pharmaceutical composition.
Embodiment 234. The method of embodiment 233, wherein a complement pathway comprises: a lectin pathway, a classical pathway or an alternative pathway.
Embodiment 235. The method of any one of embodiments 221-234, wherein administration of the pharmaceutical composition reduces and/or prevents proteinuria, decreased albumin levels and/or edema as compared to a subject who has not been administered the pharmaceutical composition or as compared to the same subject prior to administration of the pharmaceutical composition.
Embodiment 236. The method of any one of embodiments 221-235, wherein administration of the pharmaceutical composition reduces and/or prevents thickening of glomerular capillary walls as compared to a subject who has not been administered the pharmaceutical composition or as compared to the same subject prior to administration of the pharmaceutical composition.
Embodiment 237. The method of any one of embodiments 221-236, wherein administration of the pharmaceutical composition reduces and/or prevents changes in structure and/or function of podocytes as compared to a subject who has not been administered the pharmaceutical composition or as compared to the same subject prior to administration of the pharmaceutical composition.
Embodiment 238. The method of embodiment 237, wherein administration of the pharmaceutical composition prevents apoptosis in podocytes.
Embodiment 239. The method of embodiment 237 or 238, wherein administration of the pharmaceutical composition prevents an increase in intracellular calcium in human podocytes.
Embodiment 240. The method of any one of embodiments 237-239, wherein administration of the pharmaceutical composition prevents a decrease in cellular motility and/or adhesion of podocytes.
Embodiment 241. The method of any one of embodiments 216-240, wherein administration of the pharmaceutical composition treats and/or prevents the disease.
Embodiment 242. The method of any one of embodiments 216-241, wherein administration of the pharmaceutical composition alleviates one or more symptoms of the disease.
Embodiment 243. A method comprising,
assessing a level of an anti-podocyte autoantibody in a sample from a subject, and
administering a pharmaceutical composition of any one of embodiments 212 or 213,
if the level of the anti-podocyte autoantibody is higher than a comparator.
Embodiment 244. The method of embodiment 243, wherein the comparator comprises a predetermined reference sample such as a sample obtained from an otherwise similar subject who does not have a disease or disorder, or a symptom of a disease or disorder.
Embodiment 245. The method of embodiment 243 or 244, wherein the anti-podocyte autoantibody comprises: an anti-PLA2R autoantibody or a fragment or a complex thereof; an anti-THSD7A autoantibody or a fragment or a complex thereof; an anti-NELL autoantibody or a fragment or a complex thereof, or a combination thereof.
Embodiment 246. The method of any one of embodiments 243-245, wherein the level of anti-podocyte autoantibody is assessed using an ELISA.
Embodiment 247. The method of any one of embodiments 243-246, wherein the disease or disorder is iMN.
Embodiment 248. The method of any one of embodiments 243-247, wherein the method is a treatment method.
Embodiment 249. The method of any one of embodiments 243-247, wherein the method is a prevention method.
Embodiment 250. A method treating and/or preventing iMN, comprising:
(a) determining a level of an anti-podocyte autoantibody in a sample from a subject, wherein a higher level of an anti-podocyte autoantibody in the sample as compared to a comparator indicates that the subject has or is at risk of developing iMN; and
(b) responsive to said determination, administering a pharmaceutical composition of any one of embodiments 212-213 to the subject.
Embodiment 251. The method of embodiment 250, wherein the comparator comprises a predetermined reference sample such as a sample obtained from an otherwise similar subject who does not have iMN.
Embodiment 252. The method of embodiment 250 or 251, wherein the subject having a higher level of the anti-podocyte autoantibody is administered the pharmaceutical composition.
Embodiment 253. The method of any one of embodiments 250-252, wherein the anti-podocyte autoantibody comprises: an anti-PLA2R autoantibody or a fragment or a complex thereof; an anti-THSD7A autoantibody or a fragment or a complex thereof; an anti-NELL autoantibody or a fragment or a complex thereof, or a combination thereof.
Embodiment 254. The method of any one of embodiments 250-253, wherein the level of the anti-podocyte autoantibody is assessed using an ELISA.
Embodiment 255. The method of any one of embodiments 250-254, wherein the method is a treatment method.
Embodiment 256. The method of any one of embodiments 250-254, wherein the method is a prevention method.
Embodiment 257. The method of any one of embodiments 216-256, wherein the method comprises administering the pharmaceutical composition once.
Embodiment 258. The method of any one of embodiments 216-256, wherein the method comprises administering the pharmaceutical composition repeatedly.
Embodiment 259. The method of any one of embodiments 216-258, wherein the method comprises administering the pharmaceutical composition in combination with one or more additional therapies.
Embodiment 260. The method of embodiment 259, wherein the one or more additional therapies comprises a VEGF inhibitor, an anti-BAFF antibody, an anti-CD20 antibody, an anti-CD38 antibody, an anti-CD19 antibody, a Janus kinase inhibitor, a SYK inhibitor, a Factor B complement inhibitor, or a combination thereof.
Embodiment 261. The method of any one of embodiments 216-260, wherein the administration step comprises intravenous injection, intraperitoneal injection, subcutaneous injection, transdermal injection, or intramuscular injection.
Embodiment 262. The method of any one of embodiments 216-261, wherein the subject is a mammal.
Embodiment 263. The method of embodiment 262, wherein the subject is a human.

### INCORPORATION BY REFERENCE

Each publication, including scientific references to the scientific literature, patent references, and electronic databases, websites and other references accessible through the internet, are hereby incorporated by reference herein, in their entirety, for their disclosure relevant to this specification and as cited herein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A glycoengineered polypeptide comprising:
(a) a first moiety comprising one or more peptides that specifically binds to an anti-podocyte autoantibody or a fragment or a complex thereof; and
(b) a second moiety comprising one or more glycans conjugated to the first moiety at one or more glycosylation sites, wherein the anti-podocyte autoantibody is:
(i) an anti-PLA2R autoantibody or a fragment or a complex thereof;
(ii) an anti-THSD7A autoantibody or a fragment or a complex thereof;
(iii) an anti-NELL1 autoantibody or a fragment or a complex thereof;
(iv) an anti-NEP autoantibody or a fragment or a complex thereof;
(v) an anti-EXT1 autoantibody or a fragment or a complex thereof; or
(vi) an anti-EXT2 autoantibody or a fragment or a complex thereof.

2. The glycoengineered polypeptide of claim 1, wherein the glycoengineered polypeptide is capable of binding to any one, or all, or a combination of:
an anti-PLA2R autoantibody or a fragment or a complex thereof;
an anti-THSD7A autoantibody or a fragment or a complex thereof;
an anti-NELL autoantibody or a fragment or a complex thereof;
an anti-NEP autoantibody or a fragment or a complex thereof;
an anti-EXT1 autoantibody or a fragment or a complex thereof; and
an anti-EXT2 autoantibody or a fragment or a complex thereof.

3. The glycoengineered polypeptide of claim 1 or 2, wherein the second moiety specifically binds to one or more endocytic receptors chosen from: an asialoglycoprotein receptor (ASGPR); a mannose binding receptor, a Cluster of Differentiation 206 (CD206) receptor; a DC-SIGN (Cluster of Differentiation 209 or CD209) receptor; a C-Type Lectin Domain Family 4 Member G (LSECTin) receptor; a macrophage inducible Ca2+-dependent lectin receptor (Mincle); a L-SIGN CD209L receptor; dectin-1; dectin-2, langerin, macrophage mannose 2 receptor, BDCA-2, DCIR, MBL, MDL, MICL, CLEC2, CLEC10, DNGR1, CLEC12B, DEC-205, and mannose 6 phosphate receptor (M6PR), or a combination thereof.

4. The glycoengineered polypeptide of any one of the preceding claims, wherein the glycan comprises a terminal GlcNac, a terminal GalNac, or a terminal Gal.

5. The glycoengineered polypeptide of any one of the preceding claims, wherein the one or more glycans is an N-glycan, optionally wherein the N-glycan is linked to the first moiety of the glycoengineered polypeptide at 1, 2, 3, 4 or 5 N-glycosylation sites.

6. The glycoengineered polypeptide of any one of the preceding claims, wherein the one or more glycans comprise a glycan structure comprising GlcNAc2-Man3-GlcNAc2, GalNAc2-GlcNAc2-Man3-GlcNAc2, Gal2-GlcNAc2-Man3-GlcNAc2, GlcNAc1-Man3-GlcNAc2, Gal2-GlcNAc2-Man3-GlcNAc2, Gal1-GlcNAc2-Man3-GlcNAc2, GalNAc1-GlcNAc2-Man3-GlcNAc2, GlcNAc3-Man3-GlcNAc2, GlcNAc4-Man3-GlcNAc2, Gal3-GlcNAc3-Man3-GlcNAc2, GalNAc3-GlcNAc3-Man3-GlcNAc2, GalNAc4-GlcNAc4-Man3-GlcNAc2, Gal4-GlcNAc4-Man3-GlcNAc2, or Man-6-P-N-glycan.

7. The glycoengineered polypeptide of claim 6, wherein the glycan structure comprises a monoantennary structure, biantennary structure, a triantennary structure, or a tetraantennary structure.

8. The glycoengineered polypeptide of claim 6 or 7, wherein the glycan structure comprises a biantennary structure optionally, wherein the glycan structure comprises a biantennary GalNAc.

9. The glycoengineered polypeptide of claim 8, wherein the biantennary GalNac binds to an asialoglycoprotein receptor (ASGPR) or a fragment or variant thereof, or a complex comprising ASGPR.

10. The glycoengineered polypeptide of any one of claims 5-9, wherein the N-glycan has a structure of: 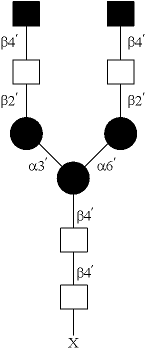
wherein the black square represents an N-acetyl galactosamine (GalNAc), the white square represents an N-acetylglucosamine (GlcNAc) residue and the black circle represents a mannose (Man) residue, and wherein X represents an amino acid residue of the first moiety.

11. The glycoengineered polypeptide of any one of claims 5-10, wherein the N-glycan is conjugated to the first moiety of the glycoengineered polypeptide at at least one, two, three, or four N-glycosylation sites.

12. The glycoengineered polypeptide of any one of claims 5-11, wherein the N-glycosylation site comprises a consensus sequence of N-X-S/T or N-X-C, wherein X is any amino acid except proline.

13. The glycoengineered polypeptide of any one of claims 5-12, wherein the N-glycosylation site is naturally occurring.

14. The glycoengineered polypeptide any one of claims 5-12, wherein the N-glycosylation site is engineered into the amino acid sequence of the first moiety.

15. The glycoengineered polypeptide of any one of claims 3-14, wherein the endocytic receptor is or comprises ASGPR or a fragment or variant thereof, or a complex comprising ASGPR, optionally, wherein when the endocytic receptor is ASGPR, the glycan structure of the second moiety comprises a terminal GalNac.

16. The glycoengineered polypeptide of any one of the preceding claims, wherein the first moiety comprises one or more peptides that specifically bind to an anti-PLA2R autoantibody or a fragment thereof.

17. The glycoengineered polypeptide of claim 16, wherein the one or more peptides that specifically bind to an anti-PLA2R autoantibody are each conjugated to a second moiety.

18. The glycoengineered polypeptide of claim 16 or 17, wherein the one or more peptides that specifically bind to an anti-PLA2R autoantibody is a soluble peptide.

19. The glycoengineered polypeptide of any one of claims 16-18, wherein the one or more peptides that specifically bind to an anti-PLA2R autoantibody comprises an epitope that is recognized by an anti-PLA2R autoantibody.

20. The glycoengineered polypeptide of any one of claims 16-19, wherein the one or more peptides that specifically bind to an anti-PLA2R autoantibody comprises a PLA2R polypeptide, or a fragment or a variant thereof.

21. The glycoengineered polypeptide of claim 20, wherein the PLA2R polypeptide is provided as SEQ ID NO: 1 (e.g., with or without the signal peptide), SEQ ID NO: 38 (e.g., with or without the signal peptide), or SEQ ID NO: 39.

22. The glycoengineered polypeptide of claim 20 or 21, wherein the one or more peptides comprise at least 5% of a full length PLA2R polypeptide, or a PLA2R polypeptide sequence provided in SEQ ID NO: 1 (e.g., with or without the signal peptide), SEQ ID NO: 38 (e.g., with or without the signal peptide), or SEQ ID NO: 39.

23. The glycoengineered polypeptide of any one of claims 20-22, wherein the one or more peptides comprise one or more domains of a PLA2R polypeptide, or fragments or variants thereof.

24. The glycoengineered polypeptide of claim 23, wherein the one or more domains of a PLA2R polypeptide include: an N-terminal cysteine rich (CysR) domain, a fibronectin 2 domain, and eight C-type lectin-like domains (CTLD), e.g., CTLD1, CTLD2, CTLD3, CTLD4, CTLD5, CTLD6, CTLD7 or CTLD8.

25. The glycoengineered polypeptide of claim 23 or 24, wherein the one or more peptides comprise:
(i) an N-terminal cysteine rich (CysR) domain, or a fragment or variant thereof;
(ii) a fibronectin 2 domain, or a fragment or variant thereof;
(iii) a CTLD1 domain, or a fragment or variant thereof;
(iv) a CTLD2 domain, or a fragment or variant thereof;
(v) a CTLD3 domain, or a fragment or variant thereof;
(vi) a CTLD4 domain, or a fragment or variant thereof;
(vii) a CTLD5 domain, or a fragment or variant thereof;
(viii) a CTLD6 domain, or a fragment or variant thereof;
(ix) a CTLD7 domain, or a fragment or variant thereof;
(x) a CTLD8 domain, or a fragment or variant thereof; or
(xi) any combination of (i)-(x).

26. The glycoengineered polypeptide of claim 25, wherein the one or more peptides comprise: an N-terminal cysteine rich (CysR) domain, or a fragment or variant thereof; an N-terminal cysteine rich (CysR) domain, or a fragment or variant thereof; a CTLD1 domain, or a fragment or variant thereof; and a CTLD2 domain, or a fragment or variant thereof.

27. The glycoengineered polypeptide of any one of claims 20-26, wherein the one or more peptides comprise a sequence having at least 85% identity to a PLA2R polypeptide sequence provided in:
(i) SEQ ID NO: 1 with or without the signal peptide of SEQ ID NO: 14,
(ii) SEQ ID NO: 38 with or without the signal peptide of SEQ ID NO: 14, or
(iii) SEQ ID NO: 39.

28. The glycoengineered polypeptide of any one of claims 16-27, wherein the one or more peptides comprises one or more additional amino acid residues on the 5′ and/or 3′ end of the sequence.

29. The glycoengineered polypeptide of any one of claims 16-28, wherein the one or more peptides that specifically bind to an anti-PLA2R autoantibody comprises a contiguous chain of amino acids comprising at least 5% of the amino acid of SEQ ID NO: 1 (e.g., with or without the signal peptide), SEQ ID NO: 38 (e.g., with or without the signal peptide), or SEQ ID NO: 39.

30. The glycoengineered polypeptide of any one of claims 16-29, wherein the one or more peptides that specifically bind to an anti-PLA2R autoantibody comprises an antibody agent comprising an antigen binding fragment.

31. The glycoengineered polypeptide of claim 30, wherein the antibody agent comprises a full antibody, a Fab fragment, an scFv, a nanobody, a duobody, or a single domain antibody (e.g., a VHH).

32. The glycoengineered polypeptide of any one of claims 1-15, wherein the first moiety comprises one or more peptides that specifically bind to an anti-NELL autoantibody or a fragment thereof.

33. The glycoengineered polypeptide of claim 32, wherein the one or more peptides that specifically bind to an anti-NELL autoantibody are each conjugated to a second moiety.

34. The glycoengineered polypeptide of claim 32 or 33, wherein the one or more peptides that specifically bind to an anti-NELL autoantibody is a soluble polypeptide.

35. The glycoengineered polypeptide of any one of claims 32-34, wherein the one or more peptides that specifically bind to an anti-NELL autoantibody comprises an epitope that is recognized by an anti-NELL autoantibody.

36. The glycoengineered polypeptide of any one of claims 32-35, wherein the one or more peptides that specifically bind to an anti-NELL autoantibody comprises a NELL polypeptide, or a fragment or a variant thereof, optionally wherein the NELL polypeptide is provided as SEQ ID NO: 9.

37. The glycoengineered polypeptide of claim 36, wherein the fragment comprises at least 5% of a full length NELL polypeptide.

38. The glycoengineered polypeptide of any one of claims 32-37, wherein the one or more peptides comprises a sequence having at least 85% identity to the amino acid sequence of SEQ ID NO: 9.

39. The glycoengineered polypeptide of any one of claims 32-38, wherein the one or more peptides that specifically bind to an anti-NELL autoantibody comprises an antibody agent comprising an antigen binding fragment.

40. The glycoengineered polypeptide of claim 39, wherein the antibody agent comprises a full antibody, a Fab fragment, an scFv, a nanobody, a duobody or a single domain antibody (e.g., a VHH).

41. The glycoengineered polypeptide of any one of claims 1-15, wherein the first moiety comprises one or more peptides that specifically bind to an anti-THSD7A autoantibody or a fragment thereof.

42. The glycoengineered polypeptide of claim 41, wherein the one or more peptides that specifically bind to an anti-THSD7A autoantibody are each conjugated to a second moiety.

43. The glycoengineered polypeptide of claim 41 or 42, wherein the one or more peptides that specifically bind to an anti-THSD7A autoantibody is a soluble polypeptide.

44. The glycoengineered polypeptide of any one of claims 41-43, wherein the one or more peptides that specifically bind to an anti-THSD7A autoantibody comprises an epitope that is recognized by an anti-THSD7A autoantibody.

45. The glycoengineered polypeptide of any one of claims 41-44, wherein the one or more peptides that specifically bind to an anti-THSD7A autoantibody comprises a THSD7A polypeptide, or a fragment or a variant thereof optionally, wherein the THSD7A polypeptide is provided as SEQ ID NO: 10.

46. The glycoengineered polypeptide of claim 45, wherein the one or more peptides that specifically bind to an anti-THSD7A autoantibody comprises a fragment of a THSD7A polypeptide, optionally wherein the fragment comprises at least 5% of a full length THSD7A polypeptide.

47. The glycoengineered polypeptide of any one of claims 41-46, wherein the one or more peptides comprise one or more domains of a THSD7A polypeptide.

48. The glycoengineered polypeptide of any one of claims 41-47, wherein the one or more peptides comprises a sequence having at least 85% identity to SEQ ID NO: 10.

49. The glycoengineered polypeptide of any one of claims 41-48, wherein the one or more peptides that specifically bind to an anti-THSD7A autoantibody comprises an antibody agent comprising an antigen binding fragment.

50. The glycoengineered polypeptide of claim 49, wherein the antibody agent comprises a full antibody, a Fab fragment, an scFv, a nanobody, a duobody, or a single domain antibody (e.g., a VHH).

51. The glycoengineered polypeptide of any the preceding claims wherein the first moiety comprises:
(i) one or more anti-PLA2R autoantibody binding polypeptides;
(ii) one or more anti-THSD7A autoantibody binding polypeptides; and/or
(iii) one or more anti-NELL autoantibody binding polypeptides;

52. The glycoengineered polypeptide of any one of the preceding claims, wherein the polypeptide comprises one or more additional elements chosen from:
(a) a linker,
(b) a spacer,
(c) a cleavage peptide, e.g., an IRES or a protease cleavage site,
(d) a signal peptide,
(e) a tag, e.g., a cleavable tag,
(f) a half-life extender domain, e.g., an Fc domain or albumin, or
(g) any combination of (a)-(f).

53. The glycoengineered polypeptide of any one of the preceding claims, wherein the second moiety is conjugated to the first moiety in vivo.

54. The glycoengineered polypeptide of claim 53, wherein the conjugation occurs in a cell, optionally wherein the cell is a Leishmania cell.

55. The glycoengineered polypeptide of any one of claims 1-52, wherein the second moiety is conjugated to the first moiety by chemical conjugation, optionally wherein chemical conjugation comprises click chemistry.

56. A polynucleotide encoding the glycoengineered polypeptide of any one of the preceding claims.

57. A composition comprising a glycoengineered polypeptide of any one of claims 1-55.

58. A composition comprising a population of glycoengineered polypeptides of any one of claims 1-55, wherein the population of glycoengineered polypeptides has an N-glycan profile that is at least 30% homogeneous at one or more of the N-glycosylation site(s).

59. The composition of claim 58, wherein the N-glycan profile comprises about 30% of the N-glycan of the structure provided in claim 10.

60. The composition of any one of claims 57-59, wherein the composition is a pharmaceutical composition.

61. A Leishmania host cell expressing a glycoengineered polypeptide of any one of claims 1-55, wherein the cell comprises a polynucleotide sequence encoding a glycoengineered polypeptide.

62. A method comprising:
administering to a subject a pharmaceutical composition of claim 60.

63. The method of claim 62, wherein the subject has or is diagnosed as having idiopathic membranous nephropathy (iMN).

64. The method of claim 62 or 63, wherein the method is a treatment method or a prevention method.

65. A method of treating and/or preventing idiopathic membranous nephropathy (iMN) in a subject, the method comprising, administering to a subject a pharmaceutical composition of claim 60.

66. The method of any one of claims 62-65, wherein the glycoengineered polypeptide is capable of simultaneously binding to an anti-podocyte autoantibody with the first moiety and binding to an endocytic receptor-expressing cell with the second moiety, thereby causing the anti-podocyte autoantibody to be internalized into a cell.

67. The method of claim 66, wherein internalization comprises transporting to a lysosome and/or degradation.

68. The method of claim 66 or 67, wherein the anti-podocyte autoantibody and the glycoengineered polypeptide are internalized.

69. The method of any one of claims 66-68, wherein:
(i) the anti-podocyte autoantibody comprises: an anti-PLA2R autoantibody or a fragment or a complex thereof; an anti-THSD7A autoantibody or a fragment or a complex thereof; an anti-NELL autoantibody or a fragment or a complex thereof, or a combination thereof; and/or
(ii) the endocytic receptor is ASGPR or a variant or fragment thereof.

70. The method of any one of claims 62-69, wherein the subject has increased levels of an anti-podocyte autoantibody as compared to a subject who does not have iMN.

71. The method of any one of claims 62-70, wherein administration of the pharmaceutical composition reduces a level of an anti-podocyte autoantibody as compared to a subject who has not been administered the pharmaceutical composition or as compared to the same subject prior to administration of the pharmaceutical composition.

72. The method of any one of claims 62-71, wherein administration of the pharmaceutical composition reduces and/or prevents formation and/or accumulation of an immune complex as compared to a subject who has not been administered the pharmaceutical composition or as compared to the same subject prior to administration of the pharmaceutical composition optionally wherein the immune complex comprises one or more: IgG antibodies, the anti-podocyte autoantibody, an antigen recognized by the anti-podocyte autoantibody, complement components, or combinations thereof.

73. The method of any one of claims 62-72, wherein administration of the pharmaceutical composition reduces activation of a complement pathway or component thereof as compared to a subject who has not been administered the pharmaceutical composition or as compared to the same subject prior to administration of the pharmaceutical composition, optionally wherein a complement pathway comprises: a lectin pathway, a classical pathway or an alternative pathway.

74. The method of any one of claims 62-73, wherein administration of the pharmaceutical composition reduces and/or prevents proteinuria, decreased albumin levels and/or edema as compared to a subject who has not been administered the pharmaceutical composition or as compared to the same subject prior to administration of the pharmaceutical composition.

75. The method of any one of claims 62-74, wherein administration of the pharmaceutical composition reduces and/or prevents thickening of glomerular capillary walls as compared to a subject who has not been administered the pharmaceutical composition or as compared to the same subject prior to administration of the pharmaceutical composition.

76. The method of any one of claims 62-75, wherein administration of the pharmaceutical composition reduces and/or prevents changes in structure and/or function of podocytes as compared to a subject who has not been administered the pharmaceutical composition or as compared to the same subject prior to administration of the pharmaceutical composition, optionally wherein administration of the pharmaceutical composition prevents apoptosis in podocytes; prevents an increase in intracellular calcium in podocytes; and/or prevents a decrease in cellular motility and/or adhesion of podocytes.

77. The method of any one of claims 62-76, wherein administration of the pharmaceutical composition treats and/or prevents the disease.

78. The method of any one of claims 62-77, wherein administration of the pharmaceutical composition alleviates one or more symptoms of the disease.

79. A method comprising,
assessing a level of an anti-podocyte autoantibody in a sample from a subject, and
administering a pharmaceutical composition of any one of claim 60,
if the level of the anti-podocyte autoantibody is higher than a comparator.

80. The method of any one of claims 62-79, wherein the method comprises administering the pharmaceutical composition in combination with one or more additional therapies, optionally wherein the one or more additional therapies comprises a VEGF inhibitor, an anti-BAFF antibody, an anti-CD20 antibody, an anti-CD38 antibody, an anti-CD19 antibody, a Janus kinase inhibitor, a SYK inhibitor, a Factor B complement inhibitor, or a combination thereof.

81. The method of any one of claims 62-80, wherein the administration step comprises intravenous injection, intraperitoneal injection, subcutaneous injection, transdermal injection, or intramuscular injection.

82. The method of any one of claims 62-81, wherein the subject is a mammal.
